SEQUENCE LISTING (337 sequences)

SEQ ID NO: 1 - protein, 472 aa, Homo sapiens
MKSILDGLADTTFRTITTDLLYVGSNDIQYEDIKGDMASKLGYFPQKFPLTSFRGSPFQEKMTAGDNPQLVPADQVNITEFYNKSLSSFKENEENIQCGENFMDIECFMVLNPSQQLAIAVLSLTLGTFTVLENLLVLCVILHSRSLRCRPSYHFIGSLAVADLLGSVIFVYSFIDFHVFHRKDSRNVFLFKLGGVTASFTASVGSLFLTAIDRYISIHRPLAYKRIVTRPKAVVAFCLMWTIAIVIAVLPLLGWNCEKLQSVCSDIFPHIDETYLMFWIGVTSVLLLFIVYAYMYILWKAHSHAVRMIQRGTQKSIIIHTSEDGKVQVTRPDQARMDIRLAKTLVLILVVLIICWGPLLAIMVYDVFGKMNKLIKTVFAFCSMLCLLNSTVNPIIYALRSKDLRHAFRSMFPSCEGTAQPLDNSMGDSDCLHKHANNAASVHRAAESCIKSTVKIAKVTMSVSTDTSAEAL

SEQ ID NO: 2 - protein, 116 aa, Homo sapiens
MKSILDGLADTTFRTITTDLLYVGSNDIQYEDIKGDMASKLGYFPQKFPLTSFRGSPFQEKMTAGDNPQLVPADQVNITEFYNKSLSSFKENEENIQCGENFMDIECFMVLNPSQQ

SEQ ID NO: 3 - protein, 26 aa, Homo sapiens
LAIAVLSLTLGTFTVLENLLVLCVIL

SEQ ID NO: 4 - protein, 12 aa, Homo sapiens
HSRSLRCRPSYH

SEQ ID NO: 5 - protein, 21 aa, Homo sapiens
FIGSLAVADLLGSVIFVYSFI

SEQ ID NO: 6 - protein, 12 aa, Homo sapiens
DFHVFHRKDSRN

SEQ ID NO: 7 - protein, 25 aa, Homo sapiens
VFLFKLGGVTASFTASVGSLFLTAI

SEQ ID NO: 8 - protein, 20 aa, Homo sapiens
DRYISIHRPLAYKRIVTRPK

SEQ ID NO: 9 - protein, 23 aa, Homo sapiens
AVVAFCLMWTIAIVIAVLPLLGW

SEQ ID NO: 10 - protein, 18 aa, Homo sapiens
NCEKLQSVCSDIFPHIDE

SEQ ID NO: 11 - protein, 26 aa, Homo sapiens
TYLMFWIGVTSVLLLFIVYAYMYILW

SEQ ID NO: 12 - protein, 45 aa, Homo sapiens
KAHSHAVRMIQRGTQKSIIIHTSEDGKVQVTRPDQARMDIRLAKT

SEQ ID NO: 13 - protein, 21 aa, Homo sapiens
LVLILVVLIICWGPLLAIMVY

SEQ ID NO: 14 - protein, 12 aa, Homo sapiens
DVFGKMNKLIKT

SEQ ID NO: 15 - protein, 22 aa, Homo sapiens
VFAFCSMLCLLNSTVNPIIYAL

SEQ ID NO: 16 - protein, 73 aa, Homo sapiens
RSKDLRHAFRSMFPSCEGTAQPLDNSMGDSDCLHKHANNAASVHRAAESCIKSTVKIAKVTMSVSTDTSAEAL

SEQ ID NO: 17 - protein, 453 aa, synthetic construct
QVQLQQSGAELVRPGVSVKISCKGSGYTFTDHALHWVKQSQARSLEWIGIISTYYGDATYNQKFKGKATMTVDKSSSTAYMELARLTSEDSAFYYCARGGLYYGTNYRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 123 aa, synthetic construct
QVQLQQSGAELVRPGVSVKISCKGSGYTFTDHALHWVKQSQARSLEWIGIISTYYGDATYNQKFKGKATMTVDKSSSTAYMELARLTSEDSAFYYCARGGLYYGTNYRAMDYWGQGTSVTVSS

SEQ ID NO: 19 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 8 aa, synthetic construct
GYTFTDHA

SEQ ID NO: 21 - protein, 9 aa, synthetic construct
IISTYYGDA

SEQ ID NO: 22 - protein, 16 aa, synthetic construct
ARGGLYYGTNYRAMDY

SEQ ID NO: 23 - protein, 214 aa, synthetic construct
DIVMTQSQKFMSTSVGDRVSVTCKASQNVGSNVAWYQQKPGQSPKALIYSASYRYSGVPDRFTGSGSGTDFTLTISNVQSEDLAEYFCQQYNNYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 107 aa, synthetic construct
DIVMTQSQKFMSTSVGDRVSVTCKASQNVGSNVAWYQQKPGQSPKALIYSASYRYSGVPDRFTGSGSGTDFTLTISNVQSEDLAEYFCQQYNNYPFTFGSGTKLEIK

SEQ ID NO: 25 - protein, 107 aa, synthetic construct [sequence omitted]

SEQ ID NO: 26 - protein, 6 aa, synthetic construct
QNVGSN

SEQ ID NO: 27 - skipped

SEQ ID NO: 28 - protein, 9 aa, synthetic construct
QQYNNYPFT

SEQ ID NO: 29 - protein, 446 aa, synthetic construct
EVQLQQSGPELVKPGTSVKISCKASGYTFTDYNMHWVKLGKSLEWIGYFYPDDGGSGYNQKFKGKATLTIDKSSSTAYMELHSLTSEDSAVYYCARGYGNSWGTYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 116 aa, synthetic construct
EVQLQQSGPELVKPGTSVKISCKASGYTFTDYNMHWVKLGKSLEWIGYFYPDDGGSGYNQKFKGKATLTIDKSSSTAYMELHSLTSEDSAVYYCARGYGNSWGTYWGQGTLVTVSA

SEQ ID NO: 31 - protein, 330 aa, synthetic construct; identical to SEQ ID NO: 19

SEQ ID NO: 32 - protein, 8 aa, synthetic construct
GYTFTDYN

SEQ ID NO: 33 - protein, 8 aa, synthetic construct
FYPDDGGS

SEQ ID NO: 34 - protein, 11 aa, synthetic construct
ARGYGNSWGTY

SEQ ID NO: 35 - protein, 215 aa, synthetic construct
EIVLTQSPTTMAASPGEKITITCSASSRISSNYLYWYQQKPGFSPKLLIYRTSNLASGVPARFSGSGSGTSYSLTIGTMEAEDVATYYCQQGSSIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 108 aa, synthetic construct
EIVLTQSPTTMAASPGEKITITCSASSRISSNYLYWYQQKPGFSPKLLIYRTSNLASGVPARFSGSGSGTSYSLTIGTMEAEDVATYYCQQGSSIPYTFGGGTKLEIK

SEQ ID NO: 37 - protein, 107 aa, synthetic construct [sequence omitted]

SEQ ID NO: 38 - protein, 7 aa, synthetic construct
SRISSNY

SEQ ID NO: 39 - skipped

SEQ ID NO: 40 - protein, 9 aa, synthetic construct
QQGSSIPYT

SEQ ID NO: 41 - protein, 453 aa, synthetic construct
QVQLQQSGPELVRPGVSVKISCKGSGYEFTDYALHWVKQSHAETLEWIGLITTYYGDTNYNQKFKGKATMTVDKSSSTAYMELARLTSEDSAIYYCARGGYYYGTDYRYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 123 aa, synthetic construct
QVQLQQSGPELVRPGVSVKISCKGSGYEFTDYALHWVKQSHAETLEWIGLITTYYGDTNYNQKFKGKATMTVDKSSSTAYMELARLTSEDSAIYYCARGGYYYGTDYRYFDVWGAGTTVTVSS

SEQ ID NO: 43 - protein, 330 aa, synthetic construct; identical to SEQ ID NO: 19

SEQ ID NO: 44 - protein, 8 aa, synthetic construct
GYEFTDYA

SEQ ID NO: 45 - protein, 8 aa, synthetic construct
ITTYYGDT

SEQ ID NO: 46 - protein, 16 aa, synthetic construct
ARGGYYYGTDYRYFDV

SEQ ID NO: 47 - protein, 214 aa, synthetic construct
DILLTQSPAILSVSPGERVSFSCRASQSIGTNIHWYQQRPNGSPRLLIKYASESISGIPSRFSGSGSGSDFTLSINSVESEDIADYYCQQSITWPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 107 aa, synthetic construct
DILLTQSPAILSVSPGERVSFSCRASQSIGTNIHWYQQRPNGSPRLLIKYASESISGIPSRFSGSGSGSDFTLSINSVESEDIADYYCQQSITWPLTFGAGTKLELK

SEQ ID NO: 49 - protein, 107 aa, synthetic construct [sequence omitted]

SEQ ID NO: 50 - protein, 6 aa, synthetic construct
QSIGTN

SEQ ID NO: 51 - skipped

SEQ ID NO: 52 - protein, 9 aa, synthetic construct
QQSITWPLT

SEQ ID NO: 53 - protein, 447 aa, synthetic construct
QVQLQQSGAELVRPGSSVKISCKASGYEFNYYWMNWVKQRPGQGLEWIGQIYPGDGDINYNGKFKGKATLTSDKSSSTVYMQLSSLTSEDSAVYFCSRSKGNPFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 117 aa, synthetic construct
QVQLQQSGAELVRPGSSVKISCKASGYEFNYYWMNWVKQRPGQGLEWIGQIYPGDGDINYNGKFKGKATLTSDKSSSTVYMQLSSLTSEDSAVYFCSRSKGNPFAYWGQGTLVTVSA

SEQ ID NO: 55 - protein, 330 aa, synthetic construct; identical to SEQ ID NO: 19

SEQ ID NO: 56 - protein, 8 aa, synthetic construct
GYEFNYYW

SEQ ID NO: 57 - protein, 8 aa, synthetic construct
IYPGDGDI

SEQ ID NO: 58 - protein, 10 aa, synthetic construct
SRSKGNPFAY

SEQ ID NO: 59 - protein, 214 aa, synthetic construct
DIQMTQTTSSLSASLGDSVTISCRASQGIRNYLNWYQQKPDGTVKLLIYYTSRLHSGVPPRFSGSGSGTDYSLTISNLEQEDLATYFCQQGNTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 107 aa, synthetic construct
DIQMTQTTSSLSASLGDSVTISCRASQGIRNYLNWYQQKPDGTVKLLIYYTSRLHSGVPPRFSGSGSGTDYSLTISNLEQEDLATYFCQQGNTLPYTFGGGTKLEIK

SEQ ID NO: 61 - protein, 107 aa, synthetic construct [sequence omitted]

SEQ ID NO: 62 - protein, 6 aa, synthetic construct
QGIRNY

SEQ ID NO: 63 - skipped

SEQ ID NO: 64 - protein, 9 aa, synthetic construct
QQGNTLPYT

SEQ ID NO: 65 - protein, 448 aa, synthetic construct
QVHLQQSGAELVRPGSSVKISCKASGYEFSYYWMNWVKQRPGQGHEWIGQIYPGDGDTNYNGKFKGKATLTADKSSSTAYMQLSSLTSEDSAVYFCARGREAAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 118 aa, synthetic construct
QVHLQQSGAELVRPGSSVKISCKASGYEFSYYWMNWVKQRPGQGHEWIGQIYPGDGDTNYNGKFKGKATLTADKSSSTAYMQLSSLTSEDSAVYFCARGREAAWFAYWGQGTLVTVSA

SEQ ID NO: 67 - protein, 330 aa, synthetic construct; identical to SEQ ID NO: 19

SEQ ID NO: 68 - protein, 8 aa, synthetic construct
GYEFSYYW

SEQ ID NO: 69 - protein, 8 aa, synthetic construct
IYPGDGDT

SEQ ID NO: 70 - protein, 11 aa, synthetic construct
ARGREAAWFAY

SEQ ID NO: 71 - protein, 213 aa, synthetic construct
DIQMTQSPASLSASVGETVTITCQASENIASDLAWYQQKQGKSPQLLVYDARNLADGVPSRFSGSGSGTHYSLNIHSLQSEDVARYYCQHYYGTPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 106 aa, synthetic construct
DIQMTQSPASLSASVGETVTITCQASENIASDLAWYQQKQGKSPQLLVYDARNLADGVPSRFSGSGSGTHYSLNIHSLQSEDVARYYCQHYYGTPTFGAGTKLELK

SEQ ID NO: 73 - protein, 107 aa, synthetic construct [sequence omitted]

SEQ ID NO: 74 - protein, 6 aa, synthetic construct
ENIASD

SEQ ID NO: 75 - skipped

SEQ ID NO: 76 - protein, 8 aa, synthetic construct
QHYYGTPT

SEQ ID NO: 77 - protein, 446 aa, synthetic construct
EVQLQQSGPELVKPGASVKISCKASGYTFTDYNLHWVKHGKSLEWIGYIYPYDGDTGYKQKFKGKATLTADKSSSTAYMELRSLTCEDSAVYYCARGYGNSWGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 116 aa, synthetic construct
EVQLQQSGPELVKPGASVKISCKASGYTFTDYNLHWVKHGKSLEWIGYIYPYDGDTGYKQKFKGKATLTADKSSSTAYMELRSLTCEDSAVYYCARGYGNSWGAYWGQGTLVTVSA

SEQ ID NO: 79 - protein, 330 aa, synthetic construct; identical to SEQ ID NO: 19

SEQ ID NO: 80 - protein, 8 aa, synthetic construct
GYTFTDYN

SEQ ID NO: 81 - protein, 8 aa, synthetic construct
IYPYDGDT

SEQ ID NO: 82 - protein, 11 aa, synthetic construct
ARGYGNSWGAY

SEQ ID NO: 83 - protein, 215 aa, synthetic construct
EIVLTQSPTTMAASPGEKITITCSATSSISSNYLHWYQQKPGFSPKLLIYRTSNLASGVPARFSGSGSGTSYSLTIGTMEAEDVATYYCQQGSSIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 108 aa, synthetic construct
EIVLTQSPTTMAASPGEKITITCSATSSISSNYLHWYQQKPGFSPKLLIYRTSNLASGVPARFSGSGSGTSYSLTIGTMEAEDVATYYCQQGSSIPYTFGGGTKLEIK

SEQ ID NO: 85 - protein, 107 aa, synthetic construct [sequence omitted]

SEQ ID NO: 86 - protein, 7 aa, synthetic construct
SSISSNY

SEQ ID NO: 87 - skipped

SEQ ID NO: 88 - protein, 8 aa, synthetic construct
QGSSIPYT

SEQ ID NO: 89 - protein, 449 aa, synthetic construct
EVQLQQPGAELVRPGASVKLSCKASSYTFTRYWMNWVKQRPEEGLEWIGMIDPYDSETHYNQKFKDKAILTVDKSSSTAYMQLSTLTSEDSAVYFCARSQPRYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 119 aa, synthetic construct
EVQLQQPGAELVRPGASVKLSCKASSYTFTRYWMNWVKQRPEEGLEWIGMIDPYDSETHYNQKFKDKAILTVDKSSSTAYMQLSTLTSEDSAVYFCARSQPRYYAMDYWGQGTSVTVSS

SEQ ID NO: 91 - protein, 330 aa, synthetic construct; identical to SEQ ID NO: 19

SEQ ID NO: 92 - protein, 8 aa, synthetic construct
SYTFTRYW

SEQ ID NO: 93 - protein, 8 aa, synthetic construct
IDPYDSET

SEQ ID NO: 94 - protein, 12 aa, synthetic construct
ARSQPRYYAMDY

SEQ ID NO: 95 - protein, 214 aa, synthetic construct
DIQMSQSPSSLSASLGERVSLTCRASQEISGFLSWLQLRPDGTIKRLIYAASSLDSGVPKRFRGSWSGSDYSLTISSLESEDFADYYCLQYSSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 107 aa, synthetic construct
DIQMSQSPSSLSASLGERVSLTCRASQEISGFLSWLQLRPDGTIKRLIYAASSLDSGVPKRFRGSWSGSDYSLTISSLESEDFADYYCLQYSSYPYTFGGGTKLEIK

SEQ ID NO: 97 - protein, 107 aa, synthetic construct [sequence omitted]

SEQ ID NO: 98 - protein, 6 aa, synthetic construct
QEISGF

SEQ ID NO: 99 - skipped

SEQ ID NO: 100 - protein, 9 aa, synthetic construct
LQYSSYPYT

SEQ ID NO: 101 - protein, 447 aa, synthetic construct
QVQLQQPGAELVKPGASVKLSCKASGYTFTDYWMHWVKQRPGHGLEWIGEIYPSSGRANYNGNFKRKATLTVDKSSSTAYMQLSSLTSEDSAVYYCARSRGNYLPYWGHGTPVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 117 aa, synthetic construct
QVQLQQPGAELVKPGASVKLSCKASGYTFTDYWMHWVKQRPGHGLEWIGEIYPSSGRANYNGNFKRKATLTVDKSSSTAYMQLSSLTSEDSAVYYCARSRGNYLPYWGHGTPVTVSA

SEQ ID NO: 103 - protein, 330 aa, synthetic construct; identical to SEQ ID NO: 19

SEQ ID NO: 104 - protein, 8 aa, synthetic construct
GYTFTDYW

SEQ ID NO: 105 - protein, 8 aa, synthetic construct
IYPSSGRA

SEQ ID NO: 106 - protein, 10 aa, synthetic construct
ARSRGNYLPY

SEQ ID NO: 107 - protein, 215 aa, synthetic construct
QIVLTQSPAIMSASLGERVTMTCTAGSTVSSSYLHWYQQRPGSSPKLWIYGTSNLASGVPARFSGSGSGTSYSLTISSMEAEDAATYYCHQYHRSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 108 aa, synthetic construct
QIVLTQSPAIMSASLGERVTMTCTAGSTVSSSYLHWYQQRPGSSPKLWIYGTSNLASGVPARFSGSGSGTSYSLTISSMEAEDAATYYCHQYHRSPPTFGGGTKLEIK

SEQ ID NO: 109 - protein, 107 aa, synthetic construct [sequence omitted]

SEQ ID NO: 110 - protein, 7 aa, synthetic construct
STVSSSY

SEQ ID NO: 111 - skipped

SEQ ID NO: 112 - protein, 9 aa, synthetic construct
HQYHRSPPT

SEQ ID NO: 113 - protein, 449 aa, synthetic construct
QVQLVQSGAEVVKPGASVKLSCKASSYTFTRYWMNWVKQAPGQGLEWIGMIDPYDSETHYNQKFKGKATLTVDKSTSTAYMELSSLRSEDTAVYFCARSQPRYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 119 aa, synthetic construct
QVQLVQSGAEVVKPGASVKLSCKASSYTFTRYWMNWVKQAPGQGLEWIGMIDPYDSETHYNQKFKGKATLTVDKSTSTAYMELSSLRSEDTAVYFCARSQPRYYAMDYWGQGTTVTVSS

SEQ ID NO: 115 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 8 aa, synthetic construct
SYTFTRYW

SEQ ID NO: 117 - protein, 8 aa, synthetic construct
IDPYDSET

SEQ ID NO: 118 - protein, 12 aa, synthetic construct
ARSQPRYYAMDY

SEQ ID NO: 119 - protein, 214 aa, synthetic construct
DIQMTQSPSSLSASLGDRVTLTCRASQEISGFLSWLQLKPGKTIKRLIYAASSLDSGVPSRFSGSWSGTDYTLTISSLQPEDFADYYCLQYSSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 107 aa, synthetic construct
DIQMTQSPSSLSASLGDRVTLTCRASQEISGFLSWLQLKPGKTIKRLIYAASSLDSGVPSRFSGSWSGTDYTLTISSLQPEDFADYYCLQYSSYPYTFGGGTKLEIK

SEQ ID NO: 121 - protein, 107 aa, synthetic construct [sequence omitted]

SEQ ID NO: 122 - protein, 6 aa, synthetic construct
QEISGF

SEQ ID NO: 123 - skipped

SEQ ID NO: 124 - protein, 9 aa, synthetic construct
LQYSSYPYT

SEQ ID NO: 125 - protein, 449 aa, synthetic construct; identical to SEQ ID NO: 113

SEQ ID NO: 126 - protein, 119 aa, synthetic construct; identical to SEQ ID NO: 114

SEQ ID NO: 127 - protein, 330 aa, synthetic construct; identical to SEQ ID NO: 115

SEQ ID NO: 128 - protein, 8 aa, synthetic construct
SYTFTRYW

SEQ ID NO: 129 - protein, 8 aa, synthetic construct
IDPYDSET

SEQ ID NO: 130 - protein, 12 aa, synthetic construct
ARSQPRYYAMDY

SEQ ID NO: 131 - protein, 214 aa, synthetic construct
DIQMTQSPSSLSASVGDRVTLTCRASQEISGFLSWLQLKPGKAIKRLIYAASSLDSGVPSRFSGSWSGTDYTLTISSLQPEDFADYYCLQYSSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 107 aa, synthetic construct
DIQMTQSPSSLSASVGDRVTLTCRASQEISGFLSWLQLKPGKAIKRLIYAASSLDSGVPSRFSGSWSGTDYTLTISSLQPEDFADYYCLQYSSYPYTFGGGTKVEIK

SEQ ID NO: 133 - protein, 107 aa, synthetic construct [sequence omitted]

SEQ ID NO: 134 - protein, 6 aa, synthetic construct
QEISGF

SEQ ID NO: 135 - skipped

SEQ ID NO: 136 - protein, 9 aa, synthetic construct
LQYSSYPYT

SEQ ID NO: 137 - protein, 449 aa, synthetic construct
QVQLVQSGAEVKKPGASVKVSCKASSYTFTRYWMNWVKQAPGQGLEWIGMIDPYDSETHYNQKFQGRATLTVDTSTSTVYMELSSLRSEDTAVYFCARSQPRYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 119 aa, synthetic construct
QVQLVQSGAEVKKPGASVKVSCKASSYTFTRYWMNWVKQAPGQGLEWIGMIDPYDSETHYNQKFQGRATLTVDTSTSTVYMELSSLRSEDTAVYFCARSQPRYYAMDYWGQGTTVTVSS

SEQ ID NO: 139 - protein, 330 aa, synthetic construct; identical to SEQ ID NO: 115

SEQ ID NO: 140 - protein, 8 aa, synthetic construct
SYTFTRYW

SEQ ID NO: 141 - protein, 8 aa, synthetic construct
IDPYDSET

SEQ ID NO: 142 - protein, 12 aa, synthetic construct
ARSQPRYYAMDY

SEQ ID NO: 143 - protein, 214 aa, synthetic construct; identical to SEQ ID NO: 119

SEQ ID NO: 144 - protein, 107 aa, synthetic construct; identical to SEQ ID NO: 120

SEQ ID NO: 145 - protein, 107 aa, synthetic construct [sequence omitted]

SEQ ID NO: 146 - protein, 6 aa, synthetic construct
QEISGF

SEQ ID NO: 147 - skipped

SEQ ID NO: 148 - protein, 9 aa, synthetic construct
LQYSSYPYT

SEQ ID NO: 149 - protein, 449 aa, synthetic construct; identical to SEQ ID NO: 137

SEQ ID NO: 150 - protein, 119 aa, synthetic construct
QVQLVQSGAEVKKPGASVKVSCKASSYTFTRYWMNWVRQAPGQGLEWMGMIDPYDSETHYNQKFKDRVTMTRDTSTSTVYMELSSLRSEDTAVYYCARSQPRYYAMDYWGQGTSVTVSS

SEQ ID NO: 151 - protein, 330 aa, synthetic construct; identical to SEQ ID NO: 115

SEQ ID NO: 152 - protein, 8 aa, synthetic construct
SYTFTRYW

SEQ ID NO: 153 - protein, 8 aa, synthetic construct
IDPYDSET

SEQ ID NO: 154 - protein, 12 aa, synthetic construct
ARSQPRYYAMDY

SEQ ID NO: 155 - protein, 214 aa, synthetic construct; identical to SEQ ID NO: 131

SEQ ID NO: 156 - protein, 107 aa, synthetic construct; identical to SEQ ID NO: 132

SEQ ID NO: 157 - protein, 107 aa, synthetic construct [sequence omitted]

SEQ ID NO: 158 - protein, 6 aa, synthetic construct
QEISGF

SEQ ID NO: 159 - skipped

SEQ ID NO: 160 - protein, 8 aa, synthetic construct
QYSSYPYT

SEQ ID NO: 161 - protein, 449 aa, synthetic construct
QVQLVQSGAEVVKPGASVKLSCKASSYTFTRYWMNWVKQAPGQGLEWIGMIDPYDSETHYNQKFKDKAILTVDKSTSTAYMELSTLRSEDTAVYFCARSQPRYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 119 aa, synthetic construct
QVQLVQSGAEVVKPGASVKLSCKASSYTFTRYWMNWVKQAPGQGLEWIGMIDPYDSETHYNQKFKDKAILTVDKSTSTAYMELSTLRSEDTAVYFCARSQPRYYAMDYWGQGTSVTVSS

SEQ ID NO: 163 - protein, 330 aa, synthetic construct; identical to SEQ ID NO: 115

SEQ ID NO: 164 - protein, 8 aa, synthetic construct
SYTFTRYW

SEQ ID NO: 165 - protein, 8 aa, synthetic construct
IDPYDSET

SEQ ID NO: 166 - protein, 12 aa, synthetic construct
ARSQPRYYAMDY

SEQ ID NO: 167 - protein, 214 aa, synthetic construct
DIQMTQSPSSLSASLGDRVTLTCRASQEISGFLSWLQLRPDGTIKRLIYAASSLDSGVPSRFRGSWSGTDYTLTISSLQSEDFADYYCLQYSSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 107 aa, synthetic construct
DIQMTQSPSSLSASLGDRVTLTCRASQEISGFLSWLQLRPDGTIKRLIYAASSLDSGVPSRFRGSWSGTDYTLTISSLQSEDFADYYCLQYSSYPYTFGGGTKLEIK

SEQ ID NO: 169 - protein, 107 aa, synthetic construct [sequence omitted]

SEQ ID NO: 170 - protein, 6 aa, synthetic construct
QEISGF

SEQ ID NO: 171 - skipped

SEQ ID NO: 172 - protein, 9 aa, synthetic construct
LQYSSYPYT

SEQ ID NO: 173 - protein, 449 aa, synthetic construct
QVQLVQSGAEVKKPGASVKVSCKASSYTFTRYWMNWVRQAPGQGLEWMGMIDPYDSETHYNQKFKDRVTMTRDTSTSTVYMELSSLRSEDTAVYYCARSQPRYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 119 aa, synthetic construct; identical to SEQ ID NO: 150

SEQ ID NO: 175 - protein, 330 aa, synthetic construct; identical to SEQ ID NO: 19

SEQ ID NO: 176 - protein, 8 aa, synthetic construct
SYTFTRYW

SEQ ID NO: 177 - protein, 8 aa, synthetic construct
IDPYDSET

SEQ ID NO: 178 - protein, 12 aa, synthetic construct
ARSQPRYYAMDY

SEQ ID NO: 179 - protein, 214 aa, synthetic construct
DIQMTQSPSSLSASVGDRVTITCRASQEISGFLAWFQQKPGKAPKSLIYAASSLQSGVPSRFSGSGSGTDFTLTISSLQPEDFATYYCQQYSSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 107 aa, synthetic construct
DIQMTQSPSSLSASVGDRVTITCRASQEISGFLAWFQQKPGKAPKSLIYAASSLQSGVPSRFSGSGSGTDFTLTISSLQPEDFATYYCQQYSSYPYTFGGGTKLEIK

SEQ ID NO: 181 - protein, 107 aa, synthetic construct [sequence omitted]

SEQ ID NO: 182 - protein, 6 aa, synthetic construct
QEISGF

SEQ ID NO: 183 - skipped

SEQ ID NO: 184 - protein, 9 aa, synthetic construct
QQYSSYPYT

SEQ ID NO: 185 - protein, 449 aa, synthetic construct; identical to SEQ ID NO: 113

SEQ ID NO: 186 - protein, 119 aa, synthetic construct; identical to SEQ ID NO: 114

SEQ ID NO: 187 - protein, 330 aa, synthetic construct; identical to SEQ ID NO: 115

SEQ ID NO: 188 - protein, 8 aa, synthetic construct
SYTFTRYW

SEQ ID NO: 189 - protein, 8 aa, synthetic construct
IDPYDSET

SEQ ID NO: 190 - protein, 12 aa, synthetic construct
ARSQPRYYAMDY

SEQ ID NO: 191 - protein, 214 aa, synthetic construct; identical to SEQ ID NO: 119

SEQ ID NO: 192 - protein, 107 aa, synthetic construct; identical to SEQ ID NO: 120

SEQ ID NO: 193 - protein, 107 aa, synthetic construct [sequence omitted]

SEQ ID NO: 194 - protein, 6 aa, synthetic construct
QEISGF

SEQ ID NO: 195 - skipped

SEQ ID NO: 196 - protein, 9 aa, synthetic construct
LQYSSYPYT

SEQ ID NO: 197 - protein, 449 aa, synthetic construct
QVQLVQSGAEVKKPGASVKLSCKASSYTFTRYWMNWVRQAPGQGLEWIGMIDPYDSETHYNQKFQGRATLTVDTSTSTAYMELSSLRSEDTAVYFCARSQPRYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 119 aa, synthetic construct
QVQLVQSGAEVKKPGASVKLSCKASSYTFTRYWMNWVRQAPGQGLEWIGMIDPYDSETHYNQKFQGRATLTVDTSTSTAYMELSSLRSEDTAVYFCARSQPRYYAMDYWGQGTTVTVSS

SEQ ID NO: 199 - protein, 330 aa, synthetic construct; identical to SEQ ID NO: 115

SEQ ID NO: 200 - protein, 8 aa, synthetic construct
SYTFTRYW

SEQ ID NO: 201 - protein, 8 aa, synthetic construct
IDPYDSET

SEQ ID NO: 202 - protein, 12 aa, synthetic construct
ARSQPRYYAMDY

SEQ ID NO: 203 - protein, 214 aa, synthetic construct; identical to SEQ ID NO: 119

SEQ ID NO: 204 - protein, 107 aa, synthetic construct; identical to SEQ ID NO: 120

SEQ ID NO: 205 - protein, 107 aa, synthetic construct [sequence omitted]

SEQ ID NO: 206 - protein, 6 aa, synthetic construct
QEISGF

SEQ ID NO: 207 - skipped

SEQ ID NO: 208 - protein, 9 aa, synthetic construct
LQYSSYPYT

SEQ ID NO: 209 - protein, 449 aa, synthetic construct
QVQLVQSGAEVKKPGASVKVSCKASSYTFTRYWMNWVRQAPGQGLEWMGMIDPYDSETHYNQKFQGRVTMTRDTSTSTVYMELSSLRSEDTAVYYCARSQPRYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 119 aa, synthetic construct
QVQLVQSGAEVKKPGASVKVSCKASSYTFTRYWMNWVRQAPGQGLEWMGMIDPYDSETHYNQKFQGRVTMTRDTSTSTVYMELSSLRSEDTAVYYCARSQPRYYAMDYWGQGTTVTVSS

SEQ ID NO: 211 - protein, 330 aa, synthetic construct; identical to SEQ ID NO: 115

SEQ ID NO: 212 - protein, 8 aa, synthetic construct
SYTFTRYW

SEQ ID NO: 213 - protein, 8 aa, synthetic construct
IDPYDSET

SEQ ID NO: 214 - protein, 12 aa, synthetic construct
ARSQPRYYAMDY

SEQ ID NO: 215 - protein, 214 aa, synthetic construct; identical to SEQ ID NO: 119

SEQ ID NO: 216 - protein, 107 aa, synthetic construct; identical to SEQ ID NO: 120

SEQ ID NO: 217 - protein, 107 aa, synthetic construct [sequence omitted]

SEQ ID NO: 218 - protein, 6 aa, synthetic construct
QEISGF

SEQ ID NO: 219 - skipped

SEQ ID NO: 220 - protein, 9 aa, synthetic construct
LQYSSYPYT

SEQ ID NO: 221 - protein, 449 aa, synthetic construct
QVQLVQSGAEVKKPGASVKVSCKASSYTFTRYWMNWVKQRPGEGLEWMGMIDPYDSETHYNQKFQGKVTMTVDKSSSTVYMELSSLRSEDTAVYFCARSQPRYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 119 aa, synthetic construct
QVQLVQSGAEVKKPGASVKVSCKASSYTFTRYWMNWVKQRPGEGLEWMGMIDPYDSETHYNQKFQGKVTMTVDKSSSTVYMELSSLRSEDTAVYFCARSQPRYYAMDYWGQGTTVTVSS

SEQ ID NO: 223 - protein, 330 aa, synthetic construct; identical to SEQ ID NO: 115

SEQ ID NO: 224 - protein, 8 aa, synthetic construct
SYTFTRYW

SEQ ID NO: 225 - protein, 8 aa, synthetic construct
IDPYDSET

SEQ ID NO: 226 - protein, 12 aa, synthetic construct
ARSQPRYYAMDY

SEQ ID NO: 227 - protein, 214 aa, synthetic construct; identical to SEQ ID NO: 119

SEQ ID NO: 228 - protein, 107 aa, synthetic construct; identical to SEQ ID NO: 120

SEQ ID NO: 229 - protein, 107 aa, synthetic construct [sequence omitted]

SEQ ID NO: 230 - protein, 6 aa, synthetic construct
QEISGF

SEQ ID NO: 231 - skipped

SEQ ID NO: 232 - protein, 9 aa, synthetic construct
LQYSSYPYT

SEQ ID NO: 233 - protein, 449 aa, synthetic construct
QVQLVQSGAEVKKPGASVKVSCKASSYTFTRYWMNWVRQAPGQGLEWMGIIDPYDSETSYAQKFQGRVTMTRDTSTSTVYMELSSLRSEDTAVYYCARSQPRYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 119 aa, synthetic construct
QVQLVQSGAEVKKPGASVKVSCKASSYTFTRYWMNWVRQAPGQGLEWMGIIDPYDSETSYAQKFQGRVTMTRDTSTSTVYMELSSLRSEDTAVYYCARSQPRYYAMDYWGQGTTVTVSS

SEQ ID NO: 235 - protein, 330 aa, synthetic construct; identical to SEQ ID NO: 115

SEQ ID NO: 236 - protein, 8 aa, synthetic construct
SYTFTRYW

SEQ ID NO: 237 - protein, 8 aa, synthetic construct
IDPYDSET

SEQ ID NO: 238 - protein, 12 aa, synthetic construct
ARSQPRYYAMDY

SEQ ID NO: 239 - protein, 214 aa, synthetic construct; identical to SEQ ID NO: 119

SEQ ID NO: 240 - protein, 107 aa, synthetic construct; identical to SEQ ID NO: 120

SEQ ID NO: 241 - protein, 107 aa, synthetic construct [sequence omitted]

SEQ ID NO: 242 - protein, 6 aa, synthetic construct
QEISGF

SEQ ID NO: 243 - skipped

SEQ ID NO: 244 - protein, 9 aa, synthetic construct
LQYSSYPYT

SEQ ID NO: 245 - protein, 449 aa, synthetic construct; identical to SEQ ID NO: 113

SEQ ID NO: 246 - protein, 119 aa, synthetic construct; identical to SEQ ID NO: 114

SEQ ID NO: 247 - protein, 330 aa, synthetic construct; identical to SEQ ID NO: 115

SEQ ID NO: 248 - protein, 8 aa, synthetic construct
SYTFTRYW

SEQ ID NO: 249 - protein, 8 aa, synthetic construct
IDPYDSET

SEQ ID NO: 250 - protein, 12 aa, synthetic construct
ARSQPRYYAMDY

SEQ ID NO: 251 - protein, 214 aa, synthetic construct
DIQMTQSPSSLSASVGDRVTLTCRASQEISGFLSWLQLKPGKAIKRLIYAASSLDSGVPSRFSGSWSGTDYTLTISSLQPEDFADYYCLQYSSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 107 aa, synthetic construct
DIQMTQSPSSLSASVGDRVTLTCRASQEISGFLSWLQLKPGKAIKRLIYAASSLDSGVPSRFSGSWSGTDYTLTISSLQPEDFADYYCLQYSSYPYTFGGGTKLEIK

SEQ ID NO: 253 - protein, 107 aa, synthetic construct [sequence omitted]

SEQ ID NO: 254 - protein, 6 aa, synthetic construct
QEISGF

SEQ ID NO: 255 - skipped

SEQ ID NO: 256 - protein, 9 aa, synthetic construct
LQYSSYPYT

SEQ ID NO: 257 - protein, 449 aa, synthetic construct; identical to SEQ ID NO: 197

SEQ ID NO: 258 - protein, 119 aa, synthetic construct; identical to SEQ ID NO: 198

SEQ ID NO: 259 - protein, 330 aa, synthetic construct; identical to SEQ ID NO: 115

SEQ ID NO: 260 - protein, 8 aa, synthetic construct
SYTFTRYW

SEQ ID NO: 261 - protein, 8 aa, synthetic construct
IDPYDSET

SEQ ID NO: 262 - protein, 12 aa, synthetic construct
ARSQPRYYAMDY

SEQ ID NO: 263 - protein, 214 aa, synthetic construct; identical to SEQ ID NO: 251

SEQ ID NO: 264 - protein, 107 aa, synthetic construct; identical to SEQ ID NO: 252

SEQ ID NO: 265 - protein, 107 aa, synthetic construct [sequence omitted]

SEQ ID NO: 266 - protein, 6 aa, synthetic construct
QEISGF

SEQ ID NO: 267 - skipped

SEQ ID NO: 268 - protein, 9 aa, synthetic construct
LQYSSYPYT

SEQ ID NO: 269 - protein, 449 aa, synthetic construct; identical to SEQ ID NO: 209

SEQ ID NO: 270 - protein, 119 aa, synthetic construct; identical to SEQ ID NO: 210

SEQ ID NO: 271 - protein, 330 aa, synthetic construct; identical to SEQ ID NO: 115

SEQ ID NO: 272 - protein, 8 aa, synthetic construct
SYTFTRYW

SEQ ID NO: 273 - protein, 8 aa, synthetic construct
IDPYDSET

SEQ ID NO: 274 - protein, 12 aa, synthetic construct
ARSQPRYYAMDY

SEQ ID NO: 275 - protein, 214 aa, synthetic construct; identical to SEQ ID NO: 251

SEQ ID NO: 276 - protein, 107 aa, synthetic construct; identical to SEQ ID NO: 252

SEQ ID NO: 277 - protein, 107 aa, synthetic construct [sequence omitted]

SEQ ID NO: 278 - protein, 6 aa, synthetic construct
QEISGF

SEQ ID NO: 279 - skipped

SEQ ID NO: 280 - protein, 9 aa, synthetic construct
LQYSSYPYT

SEQ ID NO: 281 - protein, 449 aa, synthetic construct; identical to SEQ ID NO: 221

SEQ ID NO: 282 - protein, 119 aa, synthetic construct; identical to SEQ ID NO: 222

SEQ ID NO: 283 - protein, 330 aa, synthetic construct; identical to SEQ ID NO: 115

SEQ ID NO: 284 - protein, 8 aa, synthetic construct
SYTFTRYW

SEQ ID NO: 285 - protein, 8 aa, synthetic construct
IDPYDSET

SEQ ID NO: 286 - protein, 12 aa, synthetic construct
ARSQPRYYAMDY

SEQ ID NO: 287 - protein, 214 aa, synthetic construct; identical to SEQ ID NO: 251

SEQ ID NO: 288 - protein, 107 aa, synthetic construct; identical to SEQ ID NO: 252

SEQ ID NO: 289 - protein, 107 aa, synthetic construct [sequence omitted]

SEQ ID NO: 290 - protein, 6 aa, synthetic construct
QEISGF

SEQ ID NO: 291 - skipped

SEQ ID NO: 292 - protein, 9 aa, synthetic construct
LQYSSYPYT

SEQ ID NO: 293 - protein, 449 aa, synthetic construct; identical to SEQ ID NO: 233

SEQ ID NO: 294 - protein, 119 aa, synthetic construct; identical to SEQ ID NO: 234

SEQ ID NO: 295 - protein, 330 aa, synthetic construct; identical to SEQ ID NO: 115

SEQ ID NO: 296 - protein, 8 aa, synthetic construct
SYTFTRYW

SEQ ID NO: 297 - protein, 8 aa, synthetic construct
IDPYDSET

SEQ ID NO: 298 - protein, 12 aa, synthetic construct
ARSQPRYYAMDY

SEQ ID NO: 299 - protein, 214 aa, synthetic construct; identical to SEQ ID NO: 251

SEQ ID NO: 300 - protein, 107 aa, synthetic construct; identical to SEQ ID NO: 252

SEQ ID NO: 301 - protein, 107 aa, synthetic construct [sequence omitted]

SEQ ID NO: 302 - protein, 6 aa, synthetic construct
QEISGF

SEQ ID NO: 303 - skipped

SEQ ID NO: 304 - protein, 9 aa, synthetic construct
LQYSSYPYT

SEQ ID NO: 305 - protein, 448 aa, synthetic construct
QVQLVQSGAEVVKPGASVKISCKASGYEFSYYWMNWVKQAPGQGHEWIGQIYPGDGDTNYNGKFKGKATLTADKSTSTAYMELSSLRSEDTAVYFCARGREAAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 118 aa, synthetic construct
QVQLVQSGAEVVKPGASVKISCKASGYEFSYYWMNWVKQAPGQGHEWIGQIYPGDGDTNYNGKFKGKATLTADKSTSTAYMELSSLRSEDTAVYFCARGREAAWFAYWGQGTLVTVSS

SEQ ID NO: 307 - protein, 330 aa, synthetic construct; identical to SEQ ID NO: 115

SEQ ID NO: 308 - protein, 8 aa, synthetic construct
GYEFSYYW

SEQ ID NO: 309 - protein, 8 aa, synthetic construct
IYPGDGDT

SEQ ID NO: 310 - protein, 11 aa, synthetic construct
ARGREAAWFAY

SEQ ID NO: 311 - protein, 218 aa, synthetic construct
DIQLTQSPSSLSVSVGDRATITCRASQSVSSFRYSYLHWYQQKPGKAPKLLIKYASNLESGVPSRFSGSGSGTDFTLTISSVQPEDFATYFCQHSWEIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 312 - protein, 111 aa, synthetic construct
DIQLTQSPSSLSVSVGDRATITCRASQSVSSFRYSYLHWYQQKPGKAPKLLIKYASNLESGVPSRFSGSGSGTDFTLTISSVQPEDFATYFCQHSWEIPFTFGQGTKLEIK

SEQ ID NO: 313 - protein, 107 aa, synthetic construct [sequence omitted]

SEQ ID NO: 314 - protein, 10 aa, synthetic construct
QSVSSFRYSY

SEQ ID NO: 315 - skipped

SEQ ID NO: 316 - protein, 9 aa, synthetic construct
QHSWEIPFT

SEQ ID NO: 317 - protein, 448 aa, synthetic construct
QVQLVQSGAEVKKPGASVKVSCKASGYEFSYYWMNWVRQAPGQGHEWIGQIYPGDGDTNYNQKFQGRVTLTADTSTSTAYMELSSLRSEDTAVYFCARGREAAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 118 aa, synthetic construct
QVQLVQSGAEVKKPGASVKVSCKASGYEFSYYWMNWVRQAPGQGHEWIGQIYPGDGDTNYNQKFQGRVTLTADTSTSTAYMELSSLRSEDTAVYFCARGREAAWFAYWGQGTLVTVSS

SEQ ID NO: 319 - protein, 330 aa, synthetic construct; identical to SEQ ID NO: 115

SEQ ID NO: 320 - protein, 8 aa, synthetic construct
GYEFSYYW

SEQ ID NO: 321 - protein, 8 aa, synthetic construct
IYPGDGDT

SEQ ID NO: 322 - protein, 11 aa, synthetic construct
ARGREAAWFAY

SEQ ID NO: 323 - protein, 218 aa, synthetic construct; identical to SEQ ID NO: 311

SEQ ID NO: 324 - protein, 111 aa, synthetic construct; identical to SEQ ID NO: 312

SEQ ID NO: 325 - protein, 107 aa, synthetic construct [sequence omitted]

SEQ ID NO: 326 - protein, 10 aa, synthetic construct
QSVSSFRYSY

SEQ ID NO: 327 - skipped

SEQ ID NO: 328 - protein, 9 aa, synthetic construct
QHSWEIPFT

SEQ ID NO: 329 - protein, 123 aa, synthetic construct
  VARIANT 43: note = Arg, Lys, Glu, Gln or His
  VARIANT 50: note = Ile, Tyr, Leu, Gln, Met or Glu
  VARIANT 100: note = Gly, Tyr, Lys, Arg, Gln or not present
  VARIANT 101: note = Leu, Gly, Tyr, Glu or not present
  VARIANT 102: note = Tyr, Pro or not present
  VARIANT 103: note = Tyr, Arg or not present
  VARIANT 104: note = May be deleted
  VARIANT 105: note = May be deleted
  VARIANT 106: note = Asn, Asp or not present
  VARIANT 107: note = Tyr, Asn, Gly or Ala
  VARIANT 108: note = Arg, Ser, Asn, Ala or Tyr
  VARIANT 110: note = Met, Gly, Phe or Leu
  VARIANT 111: note = Asp, Thr, Ala or Pro
QVQLQQSGAELVRPGASVKISCKASGYTFTDYWMHWVKQRPGXGLEWIGXIYPYDGDTNYNQKFKGKATLTVDKSSSTAYMQLSSLTSEDSAVYYCARGXXXXGTXXXWXXYWGQGTLVTVSA

SEQ ID NO: 330 - protein, 108 aa, synthetic construct
  VARIANT 28: note = Asn, Arg, Ser, Gly, Glu or Thr
  VARIANT 32: note = Asn, Ser or not present
  VARIANT 43: note = Gln, Phe, Gly, Lys or Ser
  VARIANT 51: note = Ser, Arg, Tyr, Asp, Ala or Gly
  VARIANT 93: note = Asn, Ser, Ile, Tyr or His
  VARIANT 95: note = Tyr, Ile, Trp, Leu, Thr or Ser
DIVLTQSPASLSASLGERVTITCRASQXISSXYLHWYQQKPGXSPKLLIYXTSNLASGVPARFSGSGSGTDYSLTISSLESEDVATYYCQQYXSXPYTFGGGTKLEIK

SEQ ID NO: 331 - protein, 114 aa, synthetic construct
QVQLVQSGAEVKKPGASVKVSCKASSYTFTRYWMNWVKQAPGQGLEWIGMIDPYDSETHYNQKFQGRATLTVDTSTSTVYMELSSLRSEDTAVYFCARSQPRYYAMDYWGQGTT

SEQ ID NO: 332 - protein, 111 aa, synthetic construct
  VARIANT 31..34: note = May be deleted
DIQMTQSPSSLSASVGDRVTLTCRASQEISSFRYGFLSWLQLKPGKAIKRLIYAASSLDSGVPSRFSGSWSGTDYTLTISSLQPEDFADYYCLQYSSYPYTFGGGTKLEIK

SEQ ID NO: 333 - protein, 219 aa, synthetic construct
DIVLAQSPASLAVSLGQRATISCRASQSVSSFRYSYLHWYQQKPGQPPRLLIKYASNLESGVPARFSGSGSGTDFTLNIHPVEEEDTATYFCQHSWEIPFTFGSGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 112 aa, synthetic construct
DIVLAQSPASLAVSLGQRATISCRASQSVSSFRYSYLHWYQQKPGQPPRLLIKYASNLESGVPARFSGSGSGTDFTLNIHPVEEEDTATYFCQHSWEIPFTFGSGTKLEIKR

SEQ ID NO: 335 - protein, 10 aa, synthetic construct
QSVSSFRYSY

SEQ ID NO: 336 - skipped

SEQ ID NO: 337 - protein, 9 aa, synthetic construct
QHSWEIPFT

(19) United States
(12) Patent Application Publication
(10) Pub. No.: US 2025/0250341 A1
(43) Pub. Date: Aug. 7, 2025

(54) CANNABINOID RECEPTOR TYPE 1 (CB1) BINDING PROTEINS AND USES THEREOF

(71) Applicant: Takeda Pharmaceutical Company, Osaka (JP)

(72) Inventors: Antara Banerjee, San Diego, CA (US); Andrea Fanjul, San Diego, CA (US); Robert J. Hoey, San Diego, CA (US); Kacey Sachen, San Diego, CA (US); Nikolai Suslov, San Diego, CA (US)

(21) Appl. No.: 19/019,913

(22) Filed: Jan. 14, 2025

Related U.S. Application Data
(63) Continuation of application No. 17/052,048, filed on Oct. 30, 2020, now Pat. No. 12,202,901.
(60) Provisional application No. 62/664,882, filed on Apr. 30, 2018.

(51) Int. Cl. C07K 16/28; A61K 39/00; A61P 3/00

(52) U.S. Cl. CPC C07K 16/286; A61P 3/00; A61K 2039/505; C07K 2317/21; C07K 2317/24; C07K 2317/33; C07K 2317/51; C07K 2317/515; C07K 2317/52; C07K 2317/524; C07K 2317/54; C07K 2317/55; C07K 2317/56; C07K 2317/565; C07K 2317/622; C07K 2317/76; C07K 2317/92

(57) ABSTRACT

The present disclosure provides isolated, engineered, non-naturally occurring CB1 binding proteins, including anti-CB1 antibodies or antigen-binding fragment thereof. The CB1 binding proteins find utility in the treatment and diagnosis of CB1 mediated conditions, diseases and disorders.

### CROSS-REFERENCE TO RELATED APPLICATIONS

This application is a continuation of U.S. patent application Ser. No. 17/052,048, filed Oct. 30, 2020; which is a U.S. national stage entry of International Patent Application No. PCT/IB2019/000503, filed Apr. 29, 2019; which claims the benefit of priority to U.S. Provisional Application No. 62/664,882, filed Apr. 30, 2018.

### SEQUENCE LISTING XML

This application contains a Sequence Listing which has been submitted electronically in XML format. The Sequence Listing XML is incorporated herein by reference. Said XML file, created on Apr. 8, 2025, is named TPX-00303_SL.xml and is 317,276 bytes in size.

### FIELD OF THE INVENTION

The invention relates to cannabinoid receptor type 1 (CB1) binding proteins and uses thereof.

### INCORPORATION BY REFERENCE

The contents of all cited references (including literature references, patents, patent applications, and websites) that may be cited throughout this application are hereby expressly incorporated by reference in their entirety for any purpose, as are the references cited therein. The disclosure will employ, unless otherwise indicated, conventional techniques of immunology, molecular biology, cell biology, drug development and drug delivery, which are well known in the art.

### BACKGROUND OF THE INVENTION

The cannabinoid receptor type 1 (CB1) is a 7-transmembrane cell membrane receptor in the G protein-coupled receptor superfamily expressed primarily in the brain, as well as peripherally in the lungs, liver, kidney, and adipose tissue. CB1 is activated by cannabinoids generated naturally inside the body called endocannabinoids (such as, eicosinoids) or cannabinoids introduced into the body (such as, cannabis), or related synthetic compounds. Cannabinoids bind reversibly and stereo-selectively to CB1. After CB1 is engaged, multiple intracellular signal transduction pathways are activated, resulting in the inhibition of adenylyl cyclase and the activation of mitogen-activated protein (MAP) kinase, the inhibition of presynaptic N- and P/Q-type calcium channels and D-type outward potassium channels, and the activation of inwardly rectifying and A-type outward potassium channels. The expression of CB1 is believed to modulate neurotransmitter release in a manner that prevents the development of excessive neuronal activity, reducing pain and other inflammatory symptoms as well as modulate food intake.

Aberrant CB1 activity has been implicated in a number of diseases, including, obesity and related disorders, such as dyslipidemia, diabetes, fibrosis, liver diseases, such as liver steatosis, kidney diseases, cardiovascular diseases and cancer.

Prader Willi Syndrome (PWS) is a genetic disorder cause by the loss of certain paternal genes and is characterized by obesity, type 2 diabetes slow development, and muscle weakness. CB-1 was validated as a target in PWS with the inverse agonist Rimonabant (Motaghedi et al. (2011) Eur. J. Med. Genet. 54: 14-18). Rimonabant (also called SR141716, Acomplia, and Zimulti) was an anorectic antiobesity drug developed and launched by Sanofi-Aventis as an oral central CB1 antagonist. The product was indicated for the treatment of obese and overweight patients with associated risk factors, such as type 2 diabetes or dyslipidemia, in conjunction with diet and exercise. In June 2006, the drug was approved for obesity by the EMEA. In 2008, Sanofi-Aventis discontinued all development and marketing of the drug for all indications, because of the risk of serious psychiatric problems, including suicidal ideation. In January 2009, the EC withdrew the drug's marketing authorization.

Another inverse agonist, taranabant (MK-0364) was investigated by Merck but its phase 3 clinical trials were stopped due to a high level of side effects including depression and anxiety. Several other CB1 inverse agonists (e.g., AM251, AM1387, and AM4113) and antagonists (e.g., cannabigerol, ibipinabant, otenabant, surinabant, tetrahydrocannabivarin, and virodhamine) have been studied, but they are either in the early stages of research or have been relegated to non-human research due to CNS side effects.

A number of CB-1 inverse agonists/antagonists are being developed that target primarily peripherally expressed CB1 by restricting their ability to cross the blood brain barrier (BBB). For example, TM-38837 is an inverse agonist/antagonist of CB1 in Phase 1 that is being developed 7TM Pharma A/S for the treatment of obesity and metabolic disorders by. Another peripherally selective silent antagonist that is not yet in the clinic is AM6545. Peripherally selective CB-1 antagonism may be a safer and more effective way to target peripheral endocannabinoid action in a number of tissues: (1) Liver—decreasing lipogenesis, fat storage, and glucose secretion; (2) Muscle—increasing glucose uptake and oxidation; (3) Adipocytes—decreasing lipogenesis and fat storage; decreasing adiponectin synthesis; and (4) gastrointestinal (GI)—increasing satiation, GI transit and absorption (Kloet and Woods (2009) Endocrinol. 150: 2531-2536).

Biologic molecules such as antibodies and related binding proteins provide a potentially safer and more effective way to deliver a therapeutic and avoid CNS involvement and side effects. Generally, only about 0.1% of circulating antibodies cross the intact BBB (Poduslo et al. (1994) Proc. Natl. Acad. Sci. USA 91: 5705-5709; Yu and Watts (2013) Neurotherapeut. 10: 459-472). Therefore, intrinsically low exposure of a functional anti-CB1 biologic to the CNS presents an opportunity to exclusively engage CB-1 peripherally, thereby limiting adverse events driven by the pharmacology of small molecules in the CNS.

Antibodies to CB1 recently have been described in the art but their advantages in the clinic are not known. See US Patent Publication Nos. 20170210797 and 20160145333.

A need therefore remains to identify a safe and effective peripherally restricted anti-CB1 inverse agonists or antagonists that does not significantly penetrate the BBB to avoid the adverse pharmacological effects of engaging CB1 in the brain.

### SUMMARY OF THE INVENTION

The invention provides binding proteins that bind cannabinoid type 1 receptor (CB1), such as antibodies and antigen-binding fragments thereof useful, in the treatment and diagnosis of disease.

An isolated antibody or antigen-binding fragment thereof that binds human cannabinoid type 1 receptor (CB1) (SEQ ID NO:1) is provided, wherein the binding protein comprises six complementarity determining regions (CDRs): CDR-H1, CDR-H2, CDR-H3, CDR-L1, CDR-L2, and CDR-L3, wherein CDR-H1 has an amino acid sequence G-Y-T-F-T-D-Y-W (residues 26-33 of SEQ ID NO:329) or a modification of said amino acid sequence by a substitution of at least one amino acid residue, wherein the substitution of G at position 1 is S; the substitution of T at position 3 is E; the substitution of T at position 5 is S or N; the substitution of D at position 6 is R or Y; the substitution of Y at position 7 is H; and the substitution of W at position 8 is A or N; CDR-H2 has an amino acid sequence I-Y-P-Y-D-G-D-T (residues 51-58 of SEQ ID NO:329) or a modification of said amino acid sequence by a substitution of at least one amino acid residue, wherein the substitution of I at position 1 is F; the substitution of Y at position 2 is D, S, or T; the substitution of P at position 3 is T; the substitution of Y at position 4 is G, D, or S; the substitution of D at position 5 is Y or S; the substitution of G at position 6 is S; the substitution of D at position 7 is E, G, or R; and the substitution of T at position 8 is A, S, or I; CDR-H3 has an amino acid sequence A-R-G-X₁-X₂-X₃-X₄-X₅-X₆-X₇-X₈-X₉-W-X₁₀-X₁₁-Y (residues 98-113 of SEQ ID NO:329) or a modification of said amino acid sequence by substitution of at least one amino acid residue, wherein the substitution of A at position 1 is S; the substitution of G at position 3 is S; X₁ at position 4 is Q, Y, K, R, or G, or is not present; X₂ at position 5 is E, Y, L or G, or is not present; X₃ at position 6 is Y or P, or is not present; X₄ at position 7 is Y, R, or E, or is not present; X₅ at position 8 is G, or is not present; X₆ at position 9 is T, or is not present; X₇ at position 10 is N or D, or is not present; X₈ at position 11 is Y, N, A, or G, or is not present; X₉ at position 12 is N, Y, S, A, or R, or is not present; the substitution of W at position 13 is Y, A, or P; X₁₀ at position 14 is L, M, F, or G, or is not present; X₁₁ at position 15 is P, D, A, or T, or is not present; the substitution of Y at position 16 is V; CDR-L1 has an amino acid sequence Q-X₁-I-S-S-X₂-Y (residues 27-33 of SEQ ID NO:330) or a modification of said amino acid sequence by substitution of at least one amino acid residue, wherein the substitution of Q at position 1 is S or E; X₁ at position 2 is E, S, T, N, G, or R; the substitution of I at position 3 is V; the substitution of S at position 4 is A, R, or G; the substitution of S at position 5 is G, N, or T; X₂ at position 6 is S, N, the peptide F-R-Y-S, or is not present; and the substitution of Y at position 7 is F, D, or N; CDR-L2 has an amino acid sequence: X₁-T-S(residues 51-53 of SEQ ID NO:330) or a modification of said amino acid sequence by substitution of at least one amino acid residue, wherein X₁ at position 1 is A, Y, G, R, D, or S; the substitution of T at position 2 is A; the substitution of S at position 3 is R; and CDR-L3 has an amino acid sequence: Q-Q-Y-X₁-S-X₂-P-Y-T(residues 91-99 of SEQ ID NO:330) or a modification of said amino acid sequence by substitution of at least one amino acid residue, wherein the substitution of Q at position 1 is L or H; the substitution of Q at position 2 is H; the substitution of Y at position 3 is S or G; X₁ at position 4 is S, W, H, Y, N, or I; the substitution of S at position 5 is E, R, G, T, or N; X₂ at position 6 is Y, I, S, T, L, or W; and the substitution of Y at position 8 is P, L, F, or is not present; and wherein said substitution, addition, or deletion of at least one amino acid residue does not inhibit the ability of said antibody or antigen-binding fragment thereof to bind human CB1.

Tables 5 and 6 provide exemplary anti-CB1 antibodies, and function antigen-binding fragments thereof of the invention.

An isolated antibody or antigen-binding fragment thereof that binds human cannabinoid type 1 receptor (CB1) (SEQ ID NO:1) is provided, wherein the antibody comprises CDRs of a variable heavy (VH) domain sequence and CDRs of a variable light (VL) domain sequence, wherein the VH domain sequence is selected from the group consisting of SEQ ID NOs: 18, 30, 42, 54, 66, 78, 90, 102, 114, 126, 138, 150, 162, 174, 186, 198, 210, 222, 234, 246, 258, 270, 282, 294, 306, and 318, and/or wherein the VL domain is selected from the group consisting of SEQ ID NOs: 24, 36, 48, 60, 72, 84, 96, 108, 120, 132, 144, 156, 168, 180, 192, 204, 216, 228, 240, 252, 264, 276, 288, 300, 312, and 324.

An isolated antibody or antigen-binding fragment thereof that binds human cannabinoid type 1 receptor (CB1) (SEQ ID NO:1) is provide, wherein the antibody comprises a variable heavy (VH) domain sequence and a variable light (VL) domain sequence, wherein the VH domain sequence is selected from the group consisting of SEQ ID NOs: 18, 30, 42, 54, 66, 78, 90, 102, 114, 126, 138, 150, 162, 174, 186, 198, 210, 222, 234, 246, 258, 270, 282, 294, 306, and 318, and/or wherein the VL domain is selected from the group consisting of SEQ ID NOs: 24, 36, 48, 60, 72, 84, 96, 108, 120, 132, 144, 156, 168, 180, 192, 204, 216, 228, 240, 252, 264, 276, 288, 300, 312, and 324.

In an embodiment, the antibody or antigen-binding fragment thereof comprises the heavy and light chain CDRs of a VH/VL pair selected from the group consisting of SEQ ID NOs: 18/24, 30/36, 42/48, 54/60, 66/72, 78/84, 90/96, 102/108, 114/120, 126/132, 138/144, 150/156, 162/168, 174/180, 186/192, 198/204, 210/216, 222/228, 234/240, 246/252, 258/264, 270/276, 282/288, 294/300, 306/312, and 318/324.

In an embodiment, the antibody or antigen-binding fragment thereof comprises the VH/VL pair selected from the group consisting of: SEQ ID NOs: 18/24, 30/36, 42/48, 54/60, 66/72, 78/84, 90/96, 102/108, 114/120, 126/132, 138/144, 150/156, 162/168, 174/180, 186/192, 198/204, 210/216, 222/228, 234/240, 246/252, 258/264, 270/276, 282/288, 294/300, 306/312, and 318/324.

In an embodiment, the antibody or antigen-binding fragment thereof comprises an HCDR set (HCDR1, HCDR2, HCDR3) selected from the group consisting of SEQ ID NOs: (20, 21, 22); (32, 33, 34); (44, 45, 46); (56, 57, 58); (68, 69, 70); (80, 81, 82); (92, 93, 94); (104, 105, 106); (116, 117, 118); (128, 129, 130); (140, 141, 142); (152, 153, 154); (164, 165, 166); (176, 177, 178); (188, 189, 190); (200, 201, 202); (212, 213, 214); (224, 225, 226); (236, 237, 238); (248, 249, 250); (260, 261, 262); (272, 273, 274); (284, 285, 286); (296, 297, 298); (308, 309, 310); and (320, 321, 322) and an LCDR set (LCDR1, LCDR2, LCDR3) selected from the group consisting of (SEQ ID NO: 26, S-A-S, SEQ ID NO: 28); (SEQ ID NO: 38, R-T-S, SEQ ID NO: 40); (SEQ ID NO: 50, Y-A-S, SEQ ID NO: 52); (SEQ ID NO: 62, Y-T-S, SEQ ID NO: 64); (SEQ ID NO: 74, D-A-R, SEQ ID NO: 76); (SEQ ID NO: 86, R-T-S, SEQ ID NO: 88); (SEQ ID NO: 98, A-A-S, SEQ ID NO: 100); (SEQ ID NO: 110, G-T-S, SEQ ID NO: 112); (SEQ ID NO: 122, A-A-S, SEQ ID NO: 124); (SEQ ID NO: 134, A-A-S, SEQ ID NO: 136); (SEQ ID NO: 146, A-A-S, SEQ ID NO: 148); (SEQ ID NO: 158, A-A-S, SEQ ID NO: 160); (SEQ ID NO: 170, A-A-S, SEQ ID NO: 172); (SEQ ID NO: 182, A-A-S, SEQ ID NO: 184); (SEQ ID NO: 194, A-A-S, SEQ ID NO: 196); (SEQ ID NO: 206, A-A-S, SEQ ID NO: 208); (SEQ ID NO: 218, A-A-S, SEQ ID NO: 220); (SEQ ID NO: 230, A-A-S, SEQ ID NO: 232); (SEQ ID NO: 242, A-A-S, SEQ ID NO: 244); (SEQ ID NO: 254, A-A-S, SEQ ID NO: 256); (SEQ ID NO: 266, A-A-S, SEQ ID NO: 268); (SEQ ID NO: 278, A-A-S, SEQ ID NO: 280); (SEQ ID NO: 290, A-A-S, SEQ ID NO: 292); (SEQ ID NO: 302, A-A-S, SEQ ID NO: 304); (SEQ ID NO: 314, Y-A-S, SEQ ID NO: 316); and (SEQ ID NO: 326, Y-A-S, SEQ ID NO: 328).

In an embodiment, the antibody or antigen-binding fragment thereof comprises the (HCDR set/LCDR set) pair selected from the group consisting of (SEQ ID NO: 20, SEQ ID NO: 21, SEQ ID NO: 22/SEQ ID NO: 26, S-A-S, SEQ ID NO: 28); (SEQ ID NO: 32, SEQ ID NO: 33, SEQ ID NO: 34/SEQ ID NO: 38, R-T-S, SEQ ID NO: 40); (SEQ ID NO: 44, SEQ ID NO: 45, SEQ ID NO: 46/SEQ ID NO: 50, Y-A-S, SEQ ID NO: 52); (SEQ ID NO: 56, SEQ ID NO: 57, SEQ ID NO: 58/SEQ ID NO: 62, Y-T-S, SEQ ID NO: 64); (SEQ ID NO: 68, SEQ ID NO: 69, SEQ ID NO: 70/SEQ ID NO: 74, D-A-R, SEQ ID NO: 76); (SEQ ID NO: 80, SEQ ID NO: 81, SEQ ID NO: 82/SEQ ID NO: 86, R-T-S, SEQ ID NO: 88); (SEQ ID NO: 92, SEQ ID NO: 93, SEQ ID NO: 94/SEQ ID NO: 98, A-A-S, SEQ ID NO: 100); (SEQ ID NO: 104, SEQ ID NO: 105, SEQ ID NO: 106/SEQ ID NO: 110, G-T-S, SEQ ID NO: 112); (SEQ ID NO: 116, SEQ ID NO: 117, SEQ ID NO: 118/SEQ ID NO: 122, A-A-S, SEQ ID NO: 124); (SEQ ID NO: 128, SEQ ID NO: 129, SEQ ID NO: 130/SEQ ID NO: 134, A-A-S, SEQ ID NO: 136); (SEQ ID NO: 140, SEQ ID NO: 141, SEQ ID NO: 142/SEQ ID NO: 146, A-A-S, SEQ ID NO: 148); (SEQ ID NO: 152, SEQ ID NO: 153, SEQ ID NO: 154/SEQ ID NO: 158, A-A-S, SEQ ID NO: 160); (SEQ ID NO: 164, SEQ ID NO: 165, SEQ ID NO: 166/SEQ ID NO: 170, A-A-S, SEQ ID NO: 172); (SEQ ID NO: 176, SEQ ID NO: 177, SEQ ID NO: 178/SEQ ID NO: 182, A-A-S, SEQ ID NO: 184); (SEQ ID NO: 188, SEQ ID NO: 189, SEQ ID NO: 190/SEQ ID NO: 194, A-A-S, SEQ ID NO: 196); (SEQ ID NO: 200, SEQ ID NO: 201, SEQ ID NO: 202/SEQ ID NO: 206, A-A-S, SEQ ID NO: 208); (SEQ ID NO: 212, SEQ ID NO: 213, SEQ ID NO: 214/SEQ ID NO: 218, A-A-S, SEQ ID NO: 220); (SEQ ID NO: 224, SEQ ID NO: 225, SEQ ID NO: 226/SEQ ID NO: 230, A-A-S, SEQ ID NO: 232); (SEQ ID NO: 236, SEQ ID NO: 237, SEQ ID NO: 238/SEQ ID NO: 242, A-A-S, SEQ ID NO: 244); (SEQ ID NO: 248, SEQ ID NO: 249, SEQ ID NO: 250/SEQ ID NO: 254, A-A-S, SEQ ID NO: 256); (SEQ ID NO: 260, SEQ ID NO: 261, SEQ ID NO: 262/SEQ ID NO: 266, A-A-S, SEQ ID NO: 268); (SEQ ID NO: 272, SEQ ID NO: 273, SEQ ID NO: 274/SEQ ID NO: 278, A-A-S, SEQ ID NO: 280); (SEQ ID NO: 284, SEQ ID NO: 285, SEQ ID NO: 286/SEQ ID NO: 290, A-A-S, SEQ ID NO: 292); (SEQ ID NO: 296, SEQ ID NO: 297, SEQ ID NO: 298/SEQ ID NO: 302, A-A-S, SEQ ID NO: 304); (SEQ ID NO: 308, SEQ ID NO: 309, SEQ ID NO: 310/SEQ ID NO: 314, Y-A-S, SEQ ID NO: 316); and (SEQ ID NO: 320, SEQ ID NO: 321, SEQ ID NO: 322/SEQ ID NO: 326, Y-A-S, SEQ ID NO: 328).

In an embodiment, the antibody or antigen-binding fragment thereof comprises the HC/LC pair selected from the group consisting of SEQ ID NOs: 17/23, 29/35, 41/47, 53/59, 65/71, 77/83, 89/95,101/107,113/119, 125/131,137/143, 149/155, 161/167, 173/179, 185/191, 197/203, 209/215, 221/227, 233/239, 245/251, 257/263, 269/275, 281/287, 283/289, 305/311, and 317/323.

An isolated antibody or antigen-binding fragment thereof that binds human cannabinoid type 1 receptor (CB1) (SEQ ID NO:1) is provided, wherein the antibody comprises CDRs of a variable heavy (VH) domain sequence and CDRs of a variable light (VL) domain sequence, wherein the VH domain sequence has at least 95% identity to an amino acid sequence selected from the group consisting of SEQ ID NOs: 18, 30, 42, 54, 66, 78, 90, 102, 114, 126, 138, 150, 162, 174, 186, 198, 210, 222, 234, 246, 258, 270, 282, 294, 306, and 318 and/or the VL domain sequence having at least 95% identity to an amino acid sequence selected from the group consisting of SEQ ID NOs: 24, 36, 48, 60, 72, 84, 96, 108, 120, 132, 144, 156, 168, 180, 192, 204, 216, 228, 240, 252, 264, 276, 288, 300, 312, and 324.

In an embodiment, the VH is set forth in SEQ ID NO: 114 and the VL is set forth in SEQ ID NO: 120. In an embodiment, the VH is set forth in SEQ ID NO: 126 and the VL is set forth in SEQ ID NO: 132. In an embodiment, the VH is set forth in SEQ ID NO: 138 and the VL is set forth in SEQ ID NO: 144. In an embodiment, the VH is set forth in SEQ ID NO: 150 and the VL is set forth in SEQ ID NO: 156. In an embodiment, the VH is set forth in SEQ ID NO: 162 and the VL is set forth in SEQ ID NO: 168. In an embodiment, the VH is set forth in SEQ ID NO: 174 and the VL is set forth in SEQ ID NO: 180. In an embodiment, the VH is set forth in SEQ ID NO: 186 and the VL is set forth in SEQ ID NO: 192. In an embodiment, the VH is set forth in SEQ ID NO: 198 and the VL is set forth in SEQ ID NO: 204. In an embodiment, the VH is set forth in SEQ ID NO: 210 and the VL is set forth in SEQ ID NO: 216. In an embodiment, the VH is set forth in SEQ ID NO: 222 and the VL is set forth in SEQ ID NO: 228. In an embodiment, the VH is set forth in SEQ ID NO: 234 and the VL is set forth in SEQ ID NO: 240. In an embodiment, the VH is set forth in SEQ ID NO: 246 and the VL is set forth in SEQ ID NO: 252. In an embodiment, the VH is set forth in SEQ ID NO: 258 and the VL is set forth in SEQ ID NO: 264. In an embodiment, the VH is set forth in SEQ ID NO: 270 and the VL is set forth in SEQ ID NO: 276. In an embodiment, the VH is set forth in SEQ ID NO: 282 and the VL is set forth in SEQ ID NO: 288. In an embodiment, the VH is set forth in SEQ ID NO: 294 and the VL is set forth in SEQ ID NO: 300. In an embodiment, the VH is set forth in SEQ ID NO: 306 and the VL is set forth in SEQ ID NO: 312. In an embodiment, the VH is set forth in SEQ ID NO: 318 and the VL is set forth in SEQ ID NO: 324.

In an embodiment, the anti-CB1 antibody is a human or humanized antibody.

In an embodiment, the anti-CB1 antigen-binding fragment comprises a Fab fragment, a Fab′ fragment, a F(ab)₂ fragment or a scFv fragment.

In an embodiment, the anti-CB1 antibody or antigen-binding fragment thereof comprises a human Fc region selected from the group consisting of an IgG1, IgG2, IgG3, IgG4, IgA, IgD, IgE, and IgM Fc.

In an embodiment, the anti-CB1 antibody or antigen-binding fragment thereof comprises a modified human Fc region selected from the group consisting of L234A/L235A (“LALA”), S228P, A330S, P331S, E233P/L234V/L235A, A327G/A330S/P331S, L234F/L235E/P331S, and N297Q.

In an embodiment, the anti-CB1 antibody or antigen-binding fragment thereof inhibits or is an antagonist of CB1 signaling activity.

In an embodiment, the anti-CB1 antibody or antigen-binding fragment thereof enhances or activates or is an agonist to CB1 signaling activity.

In an embodiment, the anti-CB1 antibody or antigen-binding fragment thereof is an inverse agonist to CB1 signaling activity.

In an embodiment, the anti-CB1 antibody or antigen-binding fragment thereof is a humanized antibody.

In an embodiment, the anti-CB1 antibody or antigen-binding fragment thereof is a fully human antibody.

Anti-CB1 antibodies or antigen-binding fragments thereof are provided that specifically bind to substantially the same epitope of CB1 as an isolated anti-human CBT antibody or antigen-binding fragment thereof.

Anti-CB1 antibodies or antigen-binding fragments thereof are provide that compete for binding to CBT with an isolated anti-human CBT antibody or antigen-binding fragment thereof.

In an embodiment, the isolated antibody or antigen-binding fragment thereof that binds to CBT has a binding affinity Kd for CBT of about 1 μM or less.

In an embodiment, the isolated antibody or antigen-binding fragment thereof that binds to CBT has a binding affinity Kd for CBT of about 100 nM or less.

In an embodiment, anti-CB1 antibody or antigen-binding fragment exhibits reduced brain penetration as compared to rimonabant.

In an embodiment, anti-CB1 antibody or antigen-binding fragment inhibits CB1 signaling that is at least 2 fold higher as compared to rimonabant.

In an embodiment, anti-CB1 antibody or antigen-binding fragment exhibits reduced CNS side effects relative to rimonabant.

An isolated nucleic acid molecule encoding an anti-CB1 antibody or antigen-binding fragment thereof is provided.

An expression vector comprising the nucleic acid molecule encoding an anti-CB1 antibody or antigen-binding fragment thereof is provided.

A host cell comprising the expression vector comprising the nucleic acid molecule encoding an anti-CB1 antibody or antigen-binding fragment thereof is provided.

A method of modulating CB1 signaling, the method comprising contacting a cell expressing CB1 with an anti-CB1 antibody or antigen-binding fragment thereof is provided.

A method of antagonizing CB1, the method comprising contacting a cell expressing CB1 with an anti-CB1 antibody or antigen-binding fragment thereof is provided.

A method of agonizing CB1, the method comprising contacting a cell expressing CB1 with an anti-CB1 antibody or antigen-binding fragment thereof is provided.

A method of inverse agonizing CB1, the method comprising contacting a cell expressing CB1 with an anti-CB1 antibody or antigen-binding fragment thereof is provided.

A pharmaceutical composition comprising an isolated anti-CB1 antibody or antigen-binding fragment thereof is provided.

A method for inhibiting the biological activity of CB1 in a subject in need thereof is provided, the method comprising administering an effective amount of the pharmaceutical composition comprising an isolated anti-CB1 antibody or antigen-binding fragment thereof to the subject thereby inhibiting the activity of the CB1 protein in the subject.

A method for treating a disease associated with CB1 activity is provided, the method comprising administering the pharmaceutical composition comprising an isolated anti-CB1 antibody or antigen-binding fragment thereof to a subject afflicted with the disease.

A method of treating a disease or disorder responsive to modulation of CB1 signaling in a subject in need thereof is provided, the method comprising administering to the pharmaceutical composition comprising an isolated anti-CB1 antibody or antigen-binding fragment thereof.

A method of treating a disease or disorder responsive to antagonism or inverse agonism of CB1 signaling in a subject in need thereof is provided, the method comprising administering to the subject the pharmaceutical composition comprising an isolated anti-CB1 antibody or antigen-binding fragment thereof.

A method of treating a disease or disorder responsive to agonism of CB1 signaling in a subject in need thereof is provided, the method comprising administering to the subject the pharmaceutical composition comprising an isolated anti-CB1 antibody or antigen-binding fragment thereof.

A method for diagnosing a disease or disorder associated with CBT is provided, the method comprising contacting a cell with an anti-CB1 antibody or antigen-binding fragment thereof.

In an embodiment, the disease or disorder is selected from the group consisting of obesity, syndromic obesities including Prader-Willi syndrome (PWS), Alström syndrome, Bardet-Biedel syndrome (BBS), Albright Hereditary Osteodystrophy (AHO), and SIM1 deletion syndrome; diabetes and related complications; dyslipidemia; liver diseases such as, for example, non-alcoholic steatohepatitis (NASH), non-alcoholic fatty liver disease, and primary biliary cirrhosis; fibrosis, for example, kidney fibrosis; chronic kidney disease; diabetic neuropathy, focal segmental glomerulosclerosis, renal disease; metabolic diseases, osteoporosis, atherosclerosis, inflammatory disease, cardiovascular disease, cancer, pain, systemic sclerosis, multiple sclerosis spasticity, glaucoma, and nicotine addiction.

In an embodiment, the disease or disorder is a kidney disease (such as Focal Segmental Glomerulosclerosis (FSGS), Diabetic nephropathy, Alport syndrome, hypertensive kidney disease, nephrotic syndrome, steroid-resistant nephrotic syndrome, minimal change disease, membranous nephropathy, idiopathic membranous nephropathy, membranoproliferative glomerulonephritis (MPGN), immune complex-mediated MPGN, complement-mediated MPGN, Lupus nephritis, postinfectious glomerulonephritis, thin basement membrane disease, mesangial proliferative glomerulonephritis, amyloidosis (primary), c1q nephropathy, rapidly progressive GN, anti-GBM disease, C3 glomerulonephritis, hypertensive nephrosclerosis, IgA nephropathy, proteinuric kidney disease, microalbuminuria, or macroalbuminuria kidney disease), pulmonary arterial hypertension, pain (such as neuropathic pain or visceral pain), cancer (such as chemoresistant breast carcinoma, adriamycin-resistant breast cancer, chemoresistant colorectal cancer, medulloblastoma, or tumor angiogenesis), anxiety, depression, transplant-related FSGS, transplant-related nephrotic syndrome, transplant-related proteinuria, cholestatic liver disease, polycystic kidney disease, autosomal dominant polycystic kidney disease (ADPKD), obesity, insulin resistance, Type II diabetes, prediabetes, metabolic syndrome, non-alcoholic fatty liver disease (NAFLD), non-alcoholic steatohepatitis (NASH), diabetic gastroparesis, or gastroparesis.

An antibody conjugate is provided comprising an isolated anti-CB1 antibody or antigen-binding fragment thereof of, wherein the antibody or antigen-binding fragment thereof is conjugated to an agent selected from the group consisting of a therapeutic agent, a cytotoxic agent, an immunoadhesion molecule, and an imaging agent.

A kit comprising the isolated anti-CB1 antibody or antigen-binding fragment thereof, and directions for the use of the antibody in an immunological assay, is provide.

### BRIEF DESCRIPTION OF THE DRAWINGS

The foregoing and other features and advantages of the present invention, as well as the invention itself, will be more fully understood from the following description of embodiments when read together with the accompanying drawings, in which:

FIG. 1 shows the results of a cAMP assay. cAMP Hunter™ CHO-K1 CNR1 Gi cells were treated with CB1 antibodies, an isotype control, or the small molecule CB1 antagonist JD5037, followed by an agonist challenge with 30 nM CP-55,940 (indicated as “Plus CP”) in the presence of forskolin. Antagonists were also tested without the addition of CP-55,940, to establish whether they had agonistic activity themselves. Key: CAB=Cell Assay Buffer, indicates assay background; CAB/F=CAB plus 15 μM forskolin, represents maximal amount of cAMP in the assay; CAB/F/CP=CAB/F plus 30 nM CP-55,940, corresponds to ˜EC80 for CP-55,940.

FIG. 2 shows the results of a p-ERK assay. Phospho/Total ERK assays were performed using cAMP Hunter™ CHO-K1 CNR1 Gi cells. Cells were treated with mouse anti-CB1 antibodies or the small molecule CB1 antagonist JD5037 followed by an agonist challenge with 30 nM CP-55,940, in the presence of forskolin. At the end of the treatment, plates were processed for p-ERK/Total ERK using the MesoScale Discovery (MSD) kit. Results are represented as a percentage of maximal response (% Max).

FIG. 3A shows the binding of mouse anti-huCB1 antibodies to huCB1-CHO cells. Binding curves of purified murine anti-CB1 antibodies were titrated 3-fold starting at 200 nM antibody concentrations. All antibodies show specific binding to the huCB1-CHO cells.

FIG. 3B shows the binding of mouse anti-huCB1 antibodies to moCB1-CHO cells. All antibodies show lack of binding to moCB1-CHO cells.

FIG. 4 shows the evaluation of binding of purified anti-CB1 antibodies in the presence and absence of agonist and antagonist CB1 small molecules CP-55,940 and JD5037, respectively, as described in Example 6.

FIG. 5A shows a consensus sequence (SEQ ID NO: 329) derived from an alignment of heavy chain variable region (VH) amino acid sequences from hybridoma antibodies M1 (SEQ ID NO: 18), M2 (SEQ ID NO: 30), M3 (SEQ ID NO: 42), M4 (SEQ ID NO: 54), M5 (SEQ ID NO: 66), M6 (SEQ ID NO: 78), M7 (SEQ ID NO: 90), and M8 (SEQ ID NO: 102).

FIG. 5B shows a consensus sequence (SEQ ID NO: 330) derived from an alignment of light chain variable region (VL) amino acid sequences from hybridoma antibodies M1(SEQ ID NO: 24), M2 (SEQ ID NO: 36), M3 (SEQ ID NO: 48), M4 (SEQ ID NO: 60), M5 (SEQ ID NO: 72), M6 (SEQ ID NO: 84), M7 (SEQ ID NO: 96), and M8 (SEQ ID NO: 108).

FIG. 6A shows a consensus sequence (SEQ ID NO: 331) derived from an alignment of heavy chain variable region (VH) amino acid sequences from humanized antibodies M7-H1 (SEQ ID NO: 114), M7-H2 (SEQ ID NO: 126), M7-H3 (SEQ ID NO: 138), M7-H4 (SEQ ID NO: 150), M7-H5 (SEQ ID NO: 162), M7-H6 (SEQ ID NO: 174), M7-H7 (SEQ ID NO: 186), M7-H8 (SEQ ID NO: 198), M7-H9 (SEQ ID NO: 210), M7-H10 (SEQ ID NO: 222), M7-H11 (SEQ ID NO: 234), M7-H12 (SEQ ID NO: 246), M7-H13 (SEQ ID NO: 258), M7-H14 (SEQ ID NO: 270), M7-H15 (SEQ ID NO: 282), M7-H16 (SEQ ID NO: 294), M5-H1 (SEQ ID NO: 306), and M5-H2 (SEQ ID NO: 318).

FIG. 6B shows a consensus sequence (SEQ ID NO: 332) derived from an alignment of light chain variable region (VL) amino acid sequences from humanized antibodies M7-H1 (SEQ ID NO: 120), M7-H2 (SEQ ID NO: 132), M7-H3 (SEQ ID NO: 144), M7-H4 (SEQ ID NO: 156), M7-H5 (SEQ ID NO: 168), M7-H6 (SEQ ID NO: 180), M7-H7 (SEQ ID NO: 192), M7-H8 (SEQ ID NO: 204), M7-H9 (SEQ ID NO: 216), M7-H10 (SEQ ID NO: 228), M7-H11 (SEQ ID NO: 240), M7-H12 (SEQ ID NO: 252), M7-H13 (SEQ ID NO: 264), M7-H14 (SEQ ID NO: 276), M7-H15 (SEQ ID NO: 288), M7-H16 (SEQ ID NO: 300), M5-H1 (SEQ ID NO: 312), and M5-H2 (SEQ ID NO: 324).

FIG. 7A displays the cell binding at a single concentration of antibody of 30 μg/mL on CHO-huCB-1 and CHO parental cells of humanized CB-1 antibody variants of clones M5 and M7.

FIG. 7B displays the cell binding at a single concentration of antibody of 30 μg/mL on CHO-huCB-1 and CHO parental cells of humanized CB-1 antibody variants of clone M7.

FIG. 8A shows the results of the cAMP assay as described in Example 3. FIG. 8A shows a side-by-side comparison between different backbones for the M5 antibody.

FIG. 8B shows the results of the cAMP assay as described in Example 3. FIG. 8B shows a side-by-side comparison between different backbones for the M7 antibody.

FIG. 9A shows the results of the p-ERK assay as described in Example 4. FIG. 9A shows a side-by-side comparison between different backbones for the M5 antibody.

FIG. 9B shows the results of the p-ERK assay as described in Example 4. FIG. 9B shows a side-by-side comparison between different backbones for the M7 antibody.

FIG. 10 shows the results of the cAMP assay for inverse agonism using the methods similar to those described in Example 3. cAMP Hunter™ CHO-K1 CNR1 Gi cells were treated with CB1 antibodies or isotype control followed by the addition of forskolin. The dotted purple line represents the level of cAMP released upon treatment with forskolin, the maximal amount of cAMP in the assay. The red line corresponds to CB1 small molecule agonist CP-55,940 used as control for the assay. Compounds having inverse agonism activity have curves going in the opposite direction to the agonist and on top of the CAB/F line. This experiment demonstrates that both M7 variants tested have inverse agonism activity.

### DETAILED DESCRIPTION OF THE INVENTION

Unless otherwise defined, scientific and technical terms used herein have the meanings that are commonly understood by those of ordinary skill in the art. In the event of any latent ambiguity, definitions provided herein take precedence over any dictionary or extrinsic definition. Unless otherwise required by context, singular terms shall include pluralities and plural terms shall include the singular. The word “a” or “an” means “at least one” unless stated otherwise. The meaning of the phrase “at least one” is equivalent to the meaning of the phrase “one or more.” The word “or” means “and/or” unless stated otherwise. As used herein, the terms “comprises,” “comprising,” “containing,” “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics are not changed, but excludes prior art embodiments. Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The methods and techniques provided herein are generally performed according to conventional methods well known in the art and as described in various general and more specific references that are cited and discussed throughout the present specification unless otherwise indicated. The nomenclatures, laboratory procedures and techniques of cell and tissue culture, molecular biology, immunology, microbiology, genetics and protein and nucleic acid chemistry and hybridization, analytical chemistry, synthetic organic chemistry, and medicinal and pharmaceutical chemistry described herein are those well-known and commonly used in the art. Standard techniques are used for enzymatic reactions and purification techniques, chemical syntheses, chemical analyses, pharmaceutical preparation, formulation, delivery, and treatment of patients.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “cannabinoid receptor type 1” or “CB1” means the 7-transmembrane cell membrane receptor encoded by the CNR1 gene and having the canonical amino acid sequence of SEQ ID NO: 1 (See https://www.uniprot.or2/uniprot/P21554). Table 1 discloses this sequence as well as each of the domains of the protein.

TABLE 1
Cannabinoid Receptor Type 1 Amino Acid Sequences

| | Amino Acid Residue | SEQ ID |
|---|---|---|
| Domain | 1234567890123456789012345678901234567890 | NO: |
| Full | MKSILDGLADTTFRTITTDLLYVGSNDIQYEDIKGDMASK | 1 |
| | LGYFPQKFPLTSFRGSPFQEKMTAGDNPQLVPADQVNITE | |
| | FYNKSLSSFKENEENIQCGENFMDIECFMVLNPSQQLAIA | |
| | VLSLTLGTFTVLENLLVLCVILHSRSLRCRPSYHFIGSLA | |
| | VADLLGSVIFVYSFIDFHVFHRKDSRNVFLFKLGGVTASF | |
| | TASVGSLFLTAIDRYISIHRPLAYKRIVTRPKAVVAFCLM | |
| | WTIAIVIAVLPLLGWNCEKLQSVCSDIFPHIDETYLMFWI | |
| | GVTSVLLLFIVYAYMYILWKAHSHAVRMIQRGTQKSIIIH | |
| | TSEDGKVQVTRPDQARMDIRLAKTLVLILVVLIICWGPLL | |
| | AIMVYDVFGKMNKL1KTVFAFCSMLCLLNSTVNPIIYALR | |
| | SKDLRHAFRSMFPSCEGTAQPLDNSMGDSDCLHKHANNAA | |
| | SVHRAAESCIKSTVKIAKVTMSVSTDTSAEAL | |
| 1 (Extracellular) | Amino Acids 1-116 of SEQ ID NO: 1 | 2 |
| 2 (Transmembrane) | Amino Acids 117-142 of SEQ ID NO: 1 | 3 |
| 3 (Cytoplasmic) | Amino Acids 143-154 of SEQ ID NO: 1 | 4 |
| 4 (Transmembrane) | Amino Acids 155-175 of SEQ ID NO: 1 | 5 |
| 5 (Extracellular) | Amino Acids 176-187 of SEQ ID NO: 1 | 6 |
| 6 (Transmembrane) | Amino Acids 188-212 of SEQ ID NO: 1 | 7 |
| 7 (Cytoplasmic) | Amino Acids 213-232 of SEQ ID NO: 1 | 8 |
| 8 (Transmembrane) | Amino Acids 233-255 of SEQ ID NO: 1 | 9 |
| 9 (Extracellular) | Amino Acids 256-273 of SEQ ID NO: 1 | 10 |
| 10 (Transmembrane) | Amino Acids 274-299 of SEQ ID NO: 1 | 11 |
| 11 (Cytoplasmic) | Amino Acids 300-344 of SEQ ID NO: 1 | 12 |
| 12 (Transmembrane) | Amino Acids 345-365 of SEQ ID NO: 1 | 13 |
| 13 (Extracellular) | Amino Acids 366-377 of SEQ ID NO: 1 | 14 |
| 14 (Transmembrane) | Amino Acids 378-399 of SEQ ID NO: 1 | 15 |
| 15 (Cytoplasmic) | Amino Acids 400-472 of SEQ ID NO: 1 | 16 |

The term “central CB1” means CB1 localized anywhere in the body, including the brain and CNS.

The term “peripheral CB1” means CB1 that is not localized to the brain or CNS (e.g., peripherally restricted CB1).

The term “antibody” means any antigen-binding molecule or molecular complex comprising at least one complementarity determining region (CDR) that specifically binds to or interacts with a particular antigen. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind CB1 specifically. Typically, such fragments would comprise an antigen-binding domain. The term “antibody” includes immunoglobulin molecules comprising four polypeptide chains, two heavy (H) chains and two light (L) chains inter-connected by disulfide bonds, as well as multimers thereof. In an embodiment of a full-length antibody, each heavy chain is comprised of a heavy chain variable region (VH) and a heavy chain constant region (CH). The CH is comprised of three domains, CH1, CH2 and CH3. Each light chain is comprised of alight chain variable region (VL) and a light chain constant region (CL). The CL is comprised of a single CL domain. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. In different embodiments of the invention, the FRs of an anti-CB1 antibody may be identical to the human germline sequences, or may be naturally or artificially modified. An amino acid consensus sequence may be defined based on a side-by-side analysis of two or more CDRs and/or FRs. Antibody molecules can be of any type (e.g., IgG, IgE, IgM, IgD, IgA and IgY), class (e.g., IgG1, IgG2, IgG3, IgG4, IgA1 and IgA2), or subclass.

The terms “HCDR set” and “LCDR set” refer to a group of three CDRs that occur in a single variable region of either a heavy or light chain, respectively, that are capable of binding an antigen. The term “(HCDR set/LCDR set) pair” refers the pairing of an HCDR set and an LCDR provide six CDRs that make up an antigen binding site. The exact boundaries of these CDRs have been defined differently according to different systems. The system described by Kabat (Kabat et al. (1987) and (1991)) not only provides an unambiguous residue numbering system applicable to any variable region of an antibody, but also provides precise residue boundaries defining the three CDRs. These CDRs may be referred to as Kabat CDRs. The term “Kabat numbering” means a system of numbering amino acid residues that are more variable (i.e., hypervariable) than other amino acid residues in the heavy and light chain variable regions of an antibody, or an antigen-binding fragment thereof (Kabat et al. (1971) Ann. NY Acad. Sci. 190: 382-391 and Kabat et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3. Chothia and coworkers (Chothia and Lesk (1987) J. Mol. Biol. 196: 901-917; Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9: 133-139 and Maccallum (1996) J. Mol. Biol. 262(5):732-45). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen-binding. The compositions and methods described herein may utilize CDRs defined according to any of these systems.

The term “VH/VL pair” refers to a VH and VL that are paired and capable of binding an antigen.

The term “HC/LC pair” refers to a HC and LC that are paired and capable of binding an antigen.

The term “Fe region” means the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (e.g., U.S. Pat. Nos. 5,648,260 and 5,624,821). The Fc region mediates several important effector functions, e.g., cytokine induction, antibody dependent cell mediated cytotoxicity (ADCC), phagocytosis, complement dependent cytotoxicity (CDC), and half-life/clearance rate of antibody and antigen-antibody complexes. In some cases these effector functions are desirable for a therapeutic immunoglobulin but in other cases might be unnecessary or even deleterious, depending on the therapeutic objectives.

The terms “antibody that binds CB1” and “anti-CB1 antibody” mean antibodies, and antigen-binding fragments thereof, that bind soluble CB1 protein or a fragment thereof (e.g., a portion of the extracellular domain of CB1) and/or cell surface-expressed CBT. The expression “cell surface-expressed CBT” means a CBT protein or portion thereof that is expressed on the surface of a cell in vitro or in vivo, such that at least a portion of the CB1 protein is exposed to the extracellular side of the cell membrane and accessible to an antigen-binding portion of an antibody.

The terms “CB1 binding protein” or “anti-CB1 binding protein” mean proteins that bind to CBT that comprise all or a portion of an antigen-binding fragment and included proteins that comprise an alternative arrangement of the typical antibody domains or framework such as a recombinant multivalent or multispecific immunoglobulins as well as conjugates and fusion proteins. The CBT binding proteins of the invention and variants and mutants thereof retain CB1 binding and function, or may provide additional or alternative functions. Such CB1 binding proteins are within the scope of the present invention and well known to those skilled in the art.

The terms “antigen-binding domain” and “antigen-binding fragment” in reference to a binding protein such as an antibody, means a portion or fragment of an antibody, or variant or mutant thereof, that retains the ability to specifically bind to the antibody's target antigen and includes any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. One or more variable and/or constant domains may be arranged into a suitable configuration, or to introduce codons, create cysteine residues, modify, add or delete amino acids, etc. Numerous fragment, mutant or variant antibody formats comprising antigen-binding fragments are known in the art. Non-limiting examples of antigen-binding fragments include: (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment comprising the VH and CH1 domains; (iv) an Fv fragment comprising the VL and VH domains of a single arm of an antibody; (v) a single-chain Fv (scFv) molecule; (vi) a dAb fragment comprising a single variable domain; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, linear antibodies (comprising a pair of tandem Fv segments; VH-CH1-VH-CH1 which form a pair of antigen binding sites with complementary light chain polypeptides), triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein. The term “antigen-binding fragment thereof” is not meant to be limiting and includes fragments contained within variant molecules that may possess additional or rearranged antibody regions, for example, multispecific antibodies and antibody conjugates that retain the same antigen binding to a particular antigen.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The term “specificity” or “specific for” in reference to a binding protein means the ability of the binding protein to selectively bind a target or antigen, e.g., with a greater affinity (i.e., a lower Kd value) than for any other target or antigen.

The antibodies of the present invention may in certain embodiments function through complement-dependent cytotoxicity (CDC) or antibody-dependent cell-mediated cytotoxicity (ADCC). CDC refers to lysis of antigen-expressing cells by an antibody of the invention in the presence of complement. ADCC refers to a cell-mediated reaction in which nonspecific cytotoxic cells that express Fc receptors (FcRs) (e.g., Natural Killer (NK) cells, neutrophils, and macrophages) recognize bound antibody on a target cell which leads to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95: 652-656).

The term “mouse antibody” means an antibody that has variable and constant regions derived from mouse germline immunoglobulin sequences. Mouse antibodies may include amino acid residues not encoded by mouse germline immunoglobulin sequences (e.g., mutations introduced by random or site-specific mutagenesis in vitro or by somatic mutation in vivo), for example in the CDRs and in particular CDR3. However, the term “mouse antibody” is not intended to include antibodies in which CDR sequences derived from the germline of another mammalian species, such as a human, have been grafted onto mouse framework sequences.

The term “recombinant antibody” means an antibody that is prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for immunoglobulin genes or antibodies prepared, expressed, created or isolated by any other means that involves splicing of immunoglobulin gene sequences to other DNA sequences. In certain embodiments, such recombinant antibodies are subjected to in vitro mutagenesis (or, when an animal transgenic for immunoglobulin sequences is used, in vivo somatic mutagenesis) and thus the amino acid sequences of the VH and VL regions of the recombinant antibodies are sequences that, while derived from and related to germline VH and VL sequences, may not naturally exist within a particular antibody germline repertoire in vivo.

The term “isolated antibody” means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “neutralizing” or “blocking” antibody means an antibody whose binding to its ligand or antigen counteracts a biological activity of the ligand or antigen. In an embodiment, the neutralizing binding protein binds to an antigen (e.g., a cytokine) and reduces its biological activity by at least about 10%, 20%, 30% 40%, 50%, 60%, 70%, 80%, 90%, 95%, 96%, 97%, 98%, 99%, or more. A neutralizing antibody that binds to CB1: (i) interferes with the interaction between CB1 or a CB1 fragment and a CB1 ligand (e.g., cannabinoid, etc.), and/or (ii) results in inhibition of at least one biological function of CB1. The inhibition caused by a CB1 neutralizing or blocking antibody need not be complete so long as it is detectable using an appropriate assay. Exemplary assays for detecting CB1 inhibition are described herein.

The term “affinity” means the strength of the interaction between a binding protein and its target antigen, and is determined by the sequence of the CDRs of the binding protein as well as by the nature of the antigen and antibody, such as their size, shape, and/or charge. Binding proteins may be selected for affinities that provide desired therapeutic end-points while minimizing negative side-effects. Affinity may be measured using methods known to one skilled in the art.

The term “affinity matured antibody” means that one or more alterations have been made in one or more CDRs or FRs thereof that result in an improvement in the affinity of the antibody for its target antigen, compared to the unaltered “parent” antibody that does not possess those alteration(s). Exemplary affinity matured antibodies will have nanomolar or even picomolar affinities for the target antigen.

Affinity matured antibodies are produced by procedures known in the art. For example, Marks et al. (1992) BioTechnology 10: 779-783 describes affinity maturation by VH and VL domain shuffling. Random mutagenesis of CDR and/or framework residues is described by Barbas et al. (1994) Proc. Nat. Acad. Sci. USA 91: 3809-3813; Schier et al. (1995) Gene 169: 147-155; Yelton et al. (1995) J. Immunol. 155: 1994-2004; Jackson et al. (1995) J. Immunol. 154(7): 3310-9; Hawkins et al. (1992) J. Mol. Biol. 226:889-896 and mutations at selective mutagenesis positions, contact or hypermutation positions with an activity enhancing amino acid residue is described in U.S. Pat. No. 6,914,128.

The term “CDR-grafted antibody” means an antibody that comprises heavy and light chain variable region sequences in which the sequences of one or more of the CDR regions of the VH and/or VL are replaced with CDR sequences of another antibody.

For example, the two antibodies can be from different species, such as antibodies having murine heavy and light chain variable regions in which one or more of the murine CDR sequences has been replaced with human CDR sequences.

The term “humanized antibody” means an antibody from a non-human species that has been altered to be more similar to human germline sequences. One type of humanized antibody is a CDR-grafted antibody, in which one or more CDR sequences are non-human and the framework region (FR) sequences are human or substantially human (e.g., they are at least 80%, at least 85%, at least 90%, at least 95%, at least 98% or at least 99% identical to) the amino acid sequence of a human antibody. A humanized antibody may comprise substantially all of at least one, and typically two, variable domains (Fab, Fab′, F(ab′)₂, FabC, Fv) in which the sequence of all or substantially all of the CDR regions correspond to those of a non-human immunoglobulin and the sequence of all or substantially all of the FR regions are those of a human immunoglobulin. The humanized antibody also may include the CH1, hinge, CH2, CH3, and CH4 regions of the heavy chain. In an embodiment, a humanized antibody also comprises at least a portion of a human immunoglobulin Fc region. In some embodiments, a humanized antibody only contains a humanized light chain. In some embodiments, a humanized antibody only contains a humanized heavy chain. In some embodiments, a humanized antibody only contains a humanized variable domain of a light chain and/or a humanized variable domain of a heavy chain. In some embodiments, a humanized antibody contains a light chain as well as at least the variable domain of a heavy chain. In some embodiments, a humanized antibody contains a heavy chain as well as at least the variable domain of a light chain.

The term “potency” means the ability of a binding protein to achieve a desired effect, and is a measurement of its therapeutic efficacy. Potency may be assessed using methods known to one skilled in the art.

The term “effective amount” means a dosage or amount that is sufficient to reduce the activity of CB1 to result in amelioration of symptoms in a patient or to achieve a desired biological outcome. Desired biological outcomes include, for example, reduction or increase of CB1 activity.

The term “cross-reactive” means the ability of a binding protein to bind a target antigen other than that against which it was raised. Generally, a binding protein will bind its target antigen with an appropriately high affinity, but can bind to the same target antigen of another species or display a low affinity for non-target antigens.

Individual binding proteins are generally selected to meet two criteria: (1) tissue staining appropriate for the known expression of the antibody target; and (2) similar staining pattern between human and toxicity study species (e.g., mouse and cynomolgus monkey) tissues from the same organ. These and other methods of assessing cross-reactivity are known to one skilled in the art.

The term “biological function” means the specific in vitro or in vivo activities of a binding protein whether present naturally or enabled by recombinant means.

Binding proteins may target several classes of antigens and achieve desired therapeutic outcomes through multiple mechanisms of action. Binding proteins may agonize, antagonize, or neutralize the activity of their targets. Binding proteins may assist in the clearance of the targets to which they bind, or may result in cytotoxicity when bound to cells. Portions of two or more antibodies may be incorporated into a multivalent format to achieve distinct functions in a single binding protein molecule. Biological activities include, but are not limited to, binding to a receptor, inducing cell proliferation, inhibiting cell growth, inducing other cytokines, inducing apoptosis, and enzymatic activity. In vitro assays and in vivo models used to assess biological function are known to one skilled in the art.

The term “stable” means able to retain its physical, chemical, and/or biological integrity or activity within a given period of time or storage conditions. A binding protein that is stable in vitro at various temperatures for an extended period of time is generally desirable. Methods of stabilizing binding proteins and assessing their stability at various temperatures are known to one skilled in the art.

The term “solubility” means the ability of a protein to remain dispersed within an aqueous solution. The solubility of a protein in an aqueous formulation depends upon the proper distribution of hydrophobic and hydrophilic amino acid residues, and therefore, solubility can correlate with the production of correctly folded proteins. A person skilled in the art will be able to detect an increase or decrease in solubility of a binding protein using routine HPLC techniques and methods known to one skilled in the art.

The term “immunogenicity” means the ability of a substance to induce an immune response. Administration of a therapeutic binding protein may result in a certain incidence of an immune response. Methods of reducing the immunogenicity of antibodies and binding proteins are known to one skilled in the art.

The term “detectable label” means a moiety attached to a member of a specific binding pair, such as an antibody or its analyte, to render a reaction (e.g., binding) between the members of the specific binding pair detectable. The labeled member of the specific binding pair is referred to as “detectably labeled.” Thus, the term “labeled binding protein” refers to a protein with a detectable label incorporated that provides for the identification of the binding protein. In an embodiment, the detectable label can produce a signal that is detectable by visual or instrumental means, e.g., incorporation of a radiolabeled amino acid or attachment to a polypeptide of biotinyl moieties that can be detected by avidin (e.g., streptavidin containing a fluorescent marker or enzymatic activity that can be detected by optical or colorimetric methods). Examples of detectable labels for polypeptides include, but are not limited to, the following: radioisotopes or radionuclides (e.g., ³H, ¹⁴C, ³⁵S ⁹⁰Y, ⁹⁹Tc, ¹¹¹In, ¹²⁵I, ¹³¹I ¹⁷⁷Lu, ¹⁶⁶Ho, or ¹⁵³Sm); chromogens; fluorescentlabels (e.g., FITC, rhodamine, lanthanide phosphors); enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates. Representative examples of labels commonly employed for immunoassays include moieties that produce light, e.g., acridinium compounds, and moieties that produce fluorescence, e.g., fluorescein. In this regard, the moiety itself may not be detectably labeled but may become detectable upon reaction with yet another moiety.

The term “conjugate” refers to a binding protein, such as an antibody, that is chemically linked to another functional molecule or second chemical moiety, such as a therapeutic agent, cytotoxic agent, cytostatic agent, or imaging agent (see for example, 7,850,962). The term “agent” includes a chemical compound, a mixture of chemical compounds, a biological macromolecule such as a peptide of protein, or an extract made from biological materials. In an embodiment, the therapeutic or cytotoxic agents include, but are not limited to, an anti-metabolite, an alkylating agent, an antibiotic, a growth factor, a cytokine, an anti-angiogenic agent, an anti-mitotic agent, an anthracycline, a toxin, and an apoptotic agent. Useful agents include, for example, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Imaging agents useful in making anti-CB1 binding protein conjugates include, but are not limited to, a radiolabel, an enzyme, a fluorescent label, a luminescent label, a bioluminescent label, a magnetic label, and biotin. When employed in the context of an immunoassay, the conjugate antibody may be a detectably labeled antibody used as the detection antibody. Antibodies can be linked by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337. Antibodies can be chemically modified by covalent conjugation to a polymer, for example, to increase their circulating half-life. Exemplary polymers and methods to attach them are also shown in U.S. Pat. Nos. 4,766,106; 4,179,337; 4,495,285, and 4,609,546.

The term “crystallized” means a binding protein that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes).

The term “vector” refers to a nucleic acid molecule capable of transporting another nucleic acid to which it has been linked. One type of vector is a “plasmid”, which refers to a circular double stranded DNA loop into which another DNA segments may be ligated. Another type of vector is a viral vector, wherein additional DNA segments may be ligated into the viral genome. Other vectors include RNA vectors. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome (e.g., non-episomal mammalian vectors). “Recombinant expression vectors” or “expression vectors” are capable of directing the expression of genes to which they are operatively linked. In the present specification, “plasmid” and “vector” may be used interchangeably as the plasmid is the most commonly used form of vector. However, other forms of expression vectors are also included, such as viral vectors (e.g., replication defective retroviruses, adenoviruses and adeno-associated viruses), which serve equivalent functions.

The terms “recombinant host cell” or “host cell” mean a cell into which exogenous DNA or RNA has been introduced. Such terms refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells. In an embodiment, eukaryotic cells include protist, fungal, plant and animal cells. In another embodiment, host cells include but are not limited to the prokaryotic cell line E. co/i; mammalian cell lines CHO, HEK 293, COS, NSO, SP2 and PER.C6; the insect cell line Sf9; and the fungal cell Saccharomyces cerevisiae.

The term “transfection” means a variety of techniques commonly used for the introduction of exogenous nucleic acid into a host cell, e.g., electroporation, calcium-phosphate precipitation, DEAE-dextran transfection and the like.

The term “biological sample” means a quantity of a substance from a living thing or formerly living thing. Such substances include, but are not limited to, blood, plasma, serum, urine, amniotic fluid, synovial fluid, endothelial cells, leukocytes, monocytes, other cells, organs, tissues, bone marrow, lymph nodes and spleen.

The term “control” refers to a composition known to either not contain analyte (“negative control”) or to contain analyte (“positive control”). A positive control can comprise a known concentration of analyte or can be used to establish assay performance characteristics and is a useful indicator of the integrity of reagents.

The term “specific binding pair” means two different molecules that specifically bind to each other through chemical or physical means. Specific binding pairs include, for example, an antibody and its antigen, biotin and avidin (or streptavidin), a carbohydrate and a lectin, complementary nucleotide sequences, effector and receptor molecules, cofactors and enzymes, enzyme and inhibitors and enzymes, and fragments and analogs thereof that retain specific binding. An example of a specific binding pair is a VH and VL region of an antibody (“VH/VL”).

The term “linker” means an amino acid residue or a polypeptide comprising two or more amino acid residues joined by peptide bonds that are used to link two polypeptides (e.g., two VH or two VL domains). Linkers are well known in the art (see, e.g., Holliger et al. (1993) Proc. Natl. Acad. Sci. USA 90: 6444-6448; Poljak et al. (1994) Structure 2: 1121-1123).

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of amino acids, saccharides, phosphoryl groups, or sulfonyl groups on the antigen and may have specific three dimensional structural characteristics, and/or specific charge characteristics. Binding proteins “bind to the same epitope” if they bind to the same amino acids on the antigen and may also cross-compete (one antibody prevents the binding or modulating effect of the other). In addition, structural definitions of epitopes (overlapping, similar, identical) are informative; and functional definitions encompass structural (binding) and functional (modulation, competition) parameters.

The term “pharmacokinetic” means the process by which a drug is absorbed, distributed, metabolized, and excreted by an organism.

The term “bioavailability” means the amount of active drug that reaches its target following administration. Bioavailability is a function of several properties, including stability, solubility, immunogenicity and pharmacokinetics, and can be assessed using methods known to one skilled in the art.

The term “surface plasmon resonance” means an optical phenomenon that allows for the analysis of real-time biospecific interactions by detection of alterations in protein concentrations within a biosensor matrix, for example using the BIAcore® system (BIAcore International AB, Uppsala, Sweden and Piscataway, NJ; Jonsson et al. (1993) Ann. Biol. Clin. 51: 19-26).

The terms “Kon,” “association rate constant,” and “Ka” mean the on rate constant for association of a binding protein (e.g., an antibody) to an antigen to form the binding protein/antigen complex. This value indicates the binding rate of a binding protein to its target antigen or the rate of complex formation between a binding protein and an antigen as shown by the following equation:

$$Antibody ( “ Ab ” ) + Antigen ( “ Ag ” ) \to “ Ab - Ag ”$$

The terms “Koff” and “dissociation rate constant” mean the off rate constant for dissociation of a binding protein (e.g., an antibody) from a binding protein/antigen complex. This value indicates the dissociation rate of a binding protein from its target antigen or separation of Ab-Ag complex over time into free antibody and antigen as shown by the following equation:

$$Ab + Ag ← Ab - Ag$$

The terms “Kd” and “equilibrium dissociation constant” mean the value obtained in a titration measurement at equilibrium, or by dividing the dissociation rate constant (Koff) by the association rate constant (Kon). Methods for determining association and dissociation rate constants are well known in the art. Fluorescence-based techniques offer high sensitivity and the ability to examine samples in physiological buffers at equilibrium. Other experimental approaches and instruments such as a BIAcore® assay (BIAcore international AB, Uppsala, Sweden) or a KinExA® assay (Sapidyne Instruments, Boise, Idaho) can be used.

The term “variant” means a polypeptide that differs from a given polypeptide in amino acid sequence by the addition, insertion, deletion, or conservative substitution of amino acids, but that retains the biological activity of the given polypeptide (e.g., a variant antibody can compete with a native antibody for binding to its target). A conservative substitution of an amino acid replaces an amino acid with a different amino acid of similar properties (e.g., hydrophilicity and degree and distribution of charged regions) and is recognized in the art as typically involving a minor change. These minor changes can be identified, in part, by considering the hydropathic index of amino acids, as is understood in the art. The hydropathic index of an amino acid is based on a consideration of its hydrophobicity and charge. Amino acids of similar hydropathic indexes in a protein can be substituted and the protein still retains protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” also includes polypeptides or fragments thereof that have been differentially processed, such as by proteolysis, phosphorylation, or other post-translational modification, yet retains biological activity or antigen reactivity. The term “variant” encompasses fragments of a variant unless otherwise defined. A variant may be 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% identical to the wildtype sequence.

The anti-CB1 antibodies disclosed herein may comprise one or more amino acid substitutions, additions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from public antibody sequence databases. The present invention includes antibodies, and antigen-binding fragments thereof, that are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The terms “substantial identity” and “substantially identical” when referring to a nucleic acid, indicates that, when optimally aligned with appropriate nucleotide insertions or deletions with another nucleic acid (or its complementary strand), there is nucleotide sequence identity, respectively, in at least about 95%, at least about 96%, at least about 97%, at least about 98% or at least about 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

The terms “substantial similarity” and “substantially similar” when referring to a polypeptide means that two polypeptide sequences, when optimally aligned, such as by the programs GAP or BESTFIT using default gap weights, share at least about 95% sequence identity, at least about 96%, at least about 97%, at least about 98% or at least about 99% sequence identity. In an embodiment, residue positions that are not identical differ by conservative amino acid substitutions. The present invention also includes anti-CB1 antibodies comprising variants of any of the VH, VL, and/or CDR amino acid sequences disclosed herein having one or more conservative substitutions. For example, the present invention includes anti-CB1 antibodies having VH, VL, and/or CDR amino acid sequences with, e.g., 10 or fewer, 8 or fewer, 6 or fewer, 4 or fewer, etc. conservative amino acid substitutions relative to any of the VH, VL, and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3)amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and Altschul et al. (1997) Nucleic Acids Res. 25: 3389-402, each herein incorporated by reference.

### Biological Characteristics of the Antibodies

The present invention includes anti-CB1 antibodies and antigen-binding fragments thereof that bind CB1 with high affinity.

Anti-CB1 antibodies and antigen-binding fragments thereof of the invention are provided that have an on rate constant (Kₒₙ) to CB1 selected from the group consisting of: at least about 10² M⁻¹s⁻¹; at least about 10³ M⁻¹s⁻¹; at least about 10⁴ M⁻¹s⁻¹; at least about 10⁵ M⁻¹ s⁻¹; and at least about 10⁶ M⁻¹s⁻¹, as measured by surface plasmon resonance.

Anti-CB1 antibodies and antigen-binding fragments thereof of the invention are provided that have an off rate constant (Koff) to said target selected from the group consisting of: at most about 10⁻³s⁻¹; at most about 10⁻⁴s⁻¹; at most about 10⁻⁵s⁻¹; and at most about 10⁻⁶s⁻¹, as measured by surface plasmon resonance.

Anti-CB1 antibodies and antigen-binding fragments thereof of the invention are provided that have a dissociation constant (KD) to said target selected from the group consisting of: at most about 10⁻⁷ M; at most about 10⁻⁸ M; at most about 10⁻⁹ M; at most about 10⁻¹⁰ M; at most about 10⁻¹¹ M; at most about 10⁻¹³ M; and at most 10⁻¹⁴ M. The anti-CB1 antibodies and fragments thereof may have a binding affinity Kd value for CB1 in the range of about 0.01 nM to about 500 nM, about 0.02 nM to about 250 nM, about 0.02 to about 200 nM, about 0.05 to about 100 nM, about 0.05 to about 50 nM. The antibodies and fragments thereof may have a binding affinity Kd value for CB1 of about 500 nM or less, about 250 nM or less, about 200 nM or less, about 150 nM or less, about 100 nM or less, about 75 nM or less, about 50 nM or less, about 25 nM or less, about 10 nM or less, about 5 nM or less, about 1 nM or less, about 500 μM or less, about 250 μM or less, about 100 μM or less, about 50 μM or less, or about 10 μM or less. In certain embodiments, the antibodies or antigen-binding fragments of the present invention bind CB1 with a Kd of less than about 15 μM, less than about 10 μM, less than about 8 μM, less than about 6 μM, less than about 4 pM, less than about 2 μM, or less than about 1 μM.

In some embodiments, the anti-CB1 antibodies or antigen-binding fragments thereof are at least as potent as small molecule CB1 receptor modulators such as, for example, rimonabant, taranabant, AM251, AM1387, AM4113, cannabigerol, ibipinabant, otenabant, surinabant, tetrahydrocannabivarin, and virodhamine, and AM6545. In some embodiments, the anti-CB1 antibodies or antigen-binding fragments thereof have CB1 antagonist or inverse agonist activity that is at least 2 fold, at least 3 fold, at least 4 fold, at least 5 fold, at least 6 fold, at least 7 fold, at least 8 fold, at least 9 fold, at least 10 fold, at least 15 fold, or at least 20 fold greater than small molecules CB1 receptor modulators such as, for example, rimonabant, taranabant, AM251, AM1387, AM4113, cannabigerol, ibipinabant, otenabant, surinabant, tetrahydrocannabivarin, and virodhamine, and AM6545. In some embodiments, the anti-CB1 antibodies or antigen-binding fragments thereof inhibit CB1 agonist-mediated signal transduction. In some embodiments, the inhibition of CB1 agonist-mediated signal transduction is measured by determining intracellular cAMP levels and/or downstream ERK phosphorylation.

In some embodiments, the anti-CB1 antibodies and antigen-binding fragments thereof have the advantage of reduced or absent BBB penetration or brain exposure. In some embodiments, the BBB penetration of the anti-CB1 antibodies and antigen-binding fragments thereof exhibit reduced brain penetration relative to small molecule CB1 agonists, antagonists, or inverse agonists (e.g., rimonabant, taranabant, AM251, AM1387, AM4113, cannabigerol, ibipinabant, otenabant, surinabant, tetrahydrocannabivarin, and virodhamine, and AM6545). In some embodiments, the anti-CB1 antibodies and antigen-binding fragments thereof provided herein provide a therapeutic benefit with reduced CNS side effects relative to a small molecule CB1 receptor agonist, antagonist, or inverse agonist. CNS side effects associated with small molecule CB1 receptor antagonist rimonabant, for example, include anxiety, depression, agitation, eating disorders, irritability, aggression, and insomnia (Moreira (2009) Rev. Bras. Psiquiatr. 31(2): 145-153).

### Epitope Mapping and Related Technologies

The present invention includes anti-CB1 antibodies that interact with one or more amino acids found within the extracellular domains of human CB1 (e.g., within amino acids 1-116, and/or extracellular loops e1 (amino acids 176-187; SEQ ID NO: 6), e2 (amino acids 256-273; SEQ ID NO: 10), and/or e3 (amino acids 366-377; SEQ ID NO: 14)). The epitope to which the antibodies bind may comprise of a single contiguous sequence of 3 or more (e.g., 3, 4, 5, 6, 7, 8, 9, 10, 11, 12, 13, 14, 15, 16, 17, 18, 19, 20 or more) amino acids located within the extracellular domain of CB1. Alternatively, the epitope may consist of a plurality of non-contiguous amino acids (or amino acid sequences) located within the extracellular domain of CB1. Further, the epitope to which the CB antibody binds may comprise a portion of CB1 that is not extracellular due to conformation change or exposure due to binding. The sequence of CB1 and its various domains is set forth in Table 1.

Various techniques known to persons of ordinary skill in the art can be used to determine whether an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., N.Y.), alanine scanning mutational analysis, peptide blots analysis (Reineke (2004) Methods Mol. Biol. 248: 443-463), and peptide cleavage analysis. In addition, methods such as epitope excision, epitope extraction and chemical modification of antigens can be employed (Tomer (2000) Protein Science 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochem. 267(2): 252-259; Engen and Smith (2001) Anal. Chem. 73: 256A-265A.

The present invention further includes anti-CB1 antibodies that bind to the same epitope as any of the specific exemplary antibodies described herein (e.g., M1, M2, M3, M4, M5 (and humanized variants thereof), M6, M7 (and humanized variants thereof), and M8). Likewise, the present invention also includes anti-CB1 antibodies that compete for binding to CB1 with any of the specific exemplary antibodies described herein (e.g., M1, M2, M3, M4, M5 (and humanized variants thereof), M6, M7 (and humanized variants thereof), and M8).

One can easily determine whether an antibody binds to the same epitope as, or competes for binding with, a reference anti-CB1 antibody by using routine methods known in the art. For example, to determine if a test antibody binds to the same epitope as a reference anti-CB1 antibody of the invention, the reference antibody is allowed to bind to a CB1 protein (e.g., a soluble portion of the CB1 extracellular domain or cell surface-expressed CB1). Next, the ability of a test antibody to bind to the CB1 molecule is assessed. If the test antibody is able to bind to CB1 following saturation binding with the reference anti-CB1 antibody, it can be concluded that the test antibody binds to a different epitope than the reference anti-CB1 antibody. On the other hand, if the test antibody is not able to bind to the CB1 molecule following saturation binding with the reference anti-CB1 antibody, then the test antibody may bind to the same epitope as the epitope bound by the reference anti-CB1 antibody of the invention. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present invention, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al. (1990) Cancer Res. 50:1495-1502). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

In some embodiments, the present invention provides an anti-CB1 antibody or antigen-binding fragment thereof that is capable of competing with the antibody or antigen-binding fragment thereof disclosed herein for binding to CB1. Such antibodies can be identified using routine competition binding assays. For example, to determine if an antibody competes for binding with a reference anti-CB1 antibody, the above-described binding methodology is performed in two orientations: In a first orientation, the reference antibody is allowed to bind to an CB1 protein (e.g., a soluble portion of the CB1extracellular domain or cell surface-expressed CB1) under saturating conditions followed by assessment of binding of the test antibody to the CB1 molecule. In a second orientation, the test antibody is allowed to bind to an CB1 molecule under saturating conditions followed by assessment of binding of the reference antibody to the CB1 molecule. If, in both orientations, only the first (saturating) antibody is capable of binding to the CB1 molecule, then it is concluded that the test antibody and the reference antibody compete for binding to CB1. An antibody that competes for binding with a reference antibody may not necessarily bind to the same epitope as the reference antibody, but may sterically block binding of the reference antibody, e.g., by binding an overlapping or adjacent epitope. Competition may be measured by ELISA, flow cytometry, or surface plasmon resonance (SPR) assay. Further, cross-competition and epitope binning assays can be performed using an Octet HTX System (Pall ForteBio LLC, Fremont, CA 94538).

In one embodiment, the anti-CB1 antibody or antigen-binding fragment thereof comprises a heavy chain CDR1 sequence having at least 80%, at least 85%, at least 90%, at least 95% at least 96%, at least 97%, at least 98%, or at least 99% identity to an amino acid sequence selected from the group consisting of SEQ ID NOs: 20, 32, 44, 56, 68, 80, 92, 104, 116, 128, 140, 152, 164, 176, 188, 200, 212, 224, 236, 248, 260, 272, 284, 296, 308, and 320. In another embodiment, the anti-CB1 antibody or antigen-binding fragment thereof comprises a heavy chain CDR2 sequence having at least 80%, at least 85%, at least 90%, at least 95% at least 96%, at least 97%, at least 98%, or at least 99% identity to an amino acid sequence selected from the group consisting of SEQ ID NOs: 21, 33, 45, 57, 69, 81, 93, 105, 117, 129, 141, 153, 165, 177, 189, 201, 213, 225, 237, 249, 261, 273, 285, 297, 309, and 321. In another embodiment, the anti-CB1 antibody or antigen-binding fragment thereof comprises a heavy chain CDR3 sequence having at least 80%, at least 85%, at least 90%, at least 95% at least 96%, at least 97%, at least 98%, or at least 99% identity to an amino acid sequence selected from the group consisting of SEQ ID NOs: 22, 34, 46, 58, 70, 82, 94, 106, 118, 130, 142, 154, 166, 178, 190, 202, 214, 226, 238, 250, 262, 274, 286, 298, 310, and 322. In another embodiment, the anti-CB1 antibody or antigen-binding fragment thereof comprises a light chain CDR1 sequence having at least 80%, at least 85%, at least 90%, at least 95% at least 96%, at least 97%, at least 98%, or at least 99% identity to an amino acid sequence selected from the group consisting of SEQ ID NOs: 26, 38, 50, 62, 74, 86, 98, 110, 122, 134, 146, 158, 170, 182, 194, 206, 218, 230, 242, 254, 266, 278, 290, 302, 314, and 326. In another embodiment, the anti-CB1 antibody or antigen-binding fragment thereof comprises a light chain CDR2 sequence having at least 80%, at least 85%, at least 90%, at least 95% at least 96%, at least 97%, at least 98%, or at least 99% identity to an amino acid sequence selected from the group consisting of S-A-S, R-T-S, Y-T-S, D-A-R, A-A-S, G-T-S, and Y-A-S. In another embodiment, the anti-CB1 antibody or antigen-binding fragment thereof comprises a light chain CDR3 sequence having at least 80%, at least 85%, at least 90%, at least 95% at least 96%, at least 97%, at least 98%, or at least 99% identity to an amino acid sequence selected from the group consisting of SEQ ID NOs: 28, 40, 52, 64, 76, 88, 100, 112, 124, 136, 148, 160, 172, 184, 196, 208, 220, 232, 244, 256, 268, 280, 292, 304, 316, and 328.

The heavy and light chain CDRs of the anti-CB1 antibodies provided herein may be independently selected and matched to form an antibody or antigen-binding fragment thereof comprising any heavy chain CDR1, CDR2, and CDR3; and any light chain CDR1, CDR2, and CDR3 from the antibodies provided herein. The heavy and light chain variable regions of the antibodies provided herein may also be independently selected and matched to form an antibody or antigen-binding fragment comprising any heavy and light chain from the antibodies provided herein.

In another embodiment, the anti-CB1 antibody or antigen-binding fragment thereof comprises a variable heavy (VH) chain sequence having at least 80%, at least 85%, at least 90%, at least 95% at least 96%, at least 97%, at least 98%, or at least 99% identity to an amino acid sequence selected from the group consisting of SEQ ID NOs: 18, 30, 42, 54, 66, 78, 90, 102, 114, 126, 138, 150, 162, 174, 186, 198, 210, 222, 234, 246, 258, 270, 282, 294, 306, and 318.

In another embodiment, the anti-CB1 antibody or antigen-binding fragment thereof comprises a variable light (VL) chain sequence having at least 80%, at least 85%, at least 90%, at least 95% at least 96%, at least 97%, at least 98%, or at least 99% identity to an amino acid sequence selected from the group consisting of SEQ ID NOs: 24, 36, 48, 60, 72, 84, 96, 108, 120, 132, 144, 156, 168, 180, 192, 204, 216, 228, 240, 252, 264, 276, 288, 300, 312, and 324.

In another embodiment, the anti-CB1 antibody or antigen-binding fragment thereof comprises a heavy chain sequence having at least 80%, at least 85%, at least 90%, at least 95% at least 96%, at least 97%, at least 98%, or at least 99% identity to an amino acid sequence selected from the group consisting of SEQ ID NOs: 17, 29, 41, 53, 65, 77, 89, 101, 113, 125, 137, 149, 161, 173, 185, 197, 209, 221, 233, 245, 257, 269, 281, 283, 305, and 317.

In another embodiment, the anti-CB1 antibody or antigen-binding fragment thereof comprises a light chain sequence having at least 80%, at least 85%, at least 90%, at least 95% at least 96%, at least 97%, at least 98%, or at least 99% identity to an amino acid sequence selected from the group consisting of SEQ ID NOs: 23, 35, 47, 59, 71, 83, 95, 107, 119, 131, 143, 155, 167, 179, 191,203,215,227,239,251,263,275,287,299,311, and 323.

In certain embodiments, the anti-CB1 antibodies or antigen-binding fragments thereof, CDRs, VH, VL, heavy chains and/or light chains comprise at least about 20%, at least about 15%, at least about 10%, at least about 5%, at least about 4%, at least about 3%, at least about 2%, or at least about 1%, conservative variant amino acids.

In another embodiment, the anti-CB1 antibody or antigen-binding fragment thereof comprises a VH CDR set having an amino acid sequences selected from the group consisting of(20, 21, 22); (32, 33, 34); (44, 45, 46); (56, 57, 58); (68, 69, 70); (80, 81, 82); (92, 93, 94); (104, 105, 106); (116, 117, 118); (128, 129, 130); (140, 141, 142); (152, 153, 154); (164, 165, 166); (176, 177, 178); (188, 189, 190); (200, 201, 202); (212, 213, 214); (224, 225, 226); (236, 237, 238); (248, 249, 250); (260, 261, 262); (272, 273, 274); (284, 285, 286); (296, 297, 298); (308, 309, 310); and (320, 321, 322).

In another embodiment, the anti-CB1 antibody or antigen-binding fragment thereof comprises a VL CDR set having an amino acid sequences selected from the group consisting of: (SEQ ID NO: 26, S-A-S, SEQ ID NO: 28); (SEQ ID NO: 38, R-T-S, SEQ ID NO: 40); (SEQ ID NO: 50, Y-A-S, SEQ ID NO: 52); (SEQ ID NO: 62, Y-T-S, SEQ ID NO: 64); (SEQ ID NO: 74, D-A-R, SEQ ID NO: 76); (SEQ ID NO: 86, R-T-S, SEQ ID NO: 88); (SEQ ID NO: 98, A-A-S, SEQ ID NO: 100); (SEQ ID NO: 110, G-T-S, SEQ ID NO: 112); (SEQ ID NO: 122, A-A-S, SEQ ID NO: 124); (SEQ ID NO: 134, A-A-S, SEQ ID NO: 136); (SEQ ID NO: 146, A-A-S, SEQ ID NO: 148); (SEQ ID NO: 158, A-A-S, SEQ ID NO: 160); (SEQ ID NO: 170, A-A-S, SEQ ID NO: 172); (SEQ ID NO: 182, A-A-S, SEQ ID NO: 184); (SEQ ID NO: 194, A-A-S, SEQ ID NO: 196); (SEQ ID NO: 206, A-A-S, SEQ ID NO: 208); (SEQ ID NO: 218, A-A-S, SEQ ID NO: 220); (SEQ ID NO: 230, A-A-S, SEQ ID NO: 232); (SEQ ID NO: 242, A-A-S, SEQ ID NO: 244); (SEQ ID NO: 254, A-A-S, SEQ ID NO: 256); (SEQ ID NO: 266, A-A-S, SEQ ID NO: 268); (SEQ ID NO: 278, A-A-S, SEQ ID NO: 280); (SEQ ID NO: 290, A-A-S, SEQ ID NO: 292); (SEQ ID NO: 302, A-A-S, SEQ ID NO: 304); (SEQ ID NO: 314, Y-A-S, SEQ ID NO: 316); and (SEQ ID NO: 326, Y-A-S, SEQ ID NO: 328).

In another embodiment, the anti-CB1 antibody or antigen-binding fragment thereof comprises a VH/VL set having an amino acid sequences selected from the group consisting of SEQ ID NOs: 18/24, 30/36, 42/48, 54/60, 66/72, 78/84, 90/96, 102/108, 114/120, 126/132, 138/144, 150/156, 162/168, 174/180, 186/192, 198/204, 210/216, 222/228, 234/240, 246/252, 258/264, 270/276, 282/288, 294/300, 306/312, and 318/324.

In another embodiment, the anti-CB1 antibody or antigen-binding fragment thereof comprises a heavy and light chain set having an amino acid sequences selected from the group consisting of SEQ ID NOs: SEQ ID NOs: 17/23, 29/35, 41/47, 53/59, 65/71, 77/83, 89/95, 101/107, 113/119, 125/131, 137/143, 149/155, 161/167, 173/179, 185/191, 197/203, 209/215, 221/227, 233/239, 245/251, 257/263, 269/275, 281/287, 283/289, 305/311, and 317/323.

In some embodiments, the anti-CB1 antibody or antigen-binding fragment thereof binds CB1 and exhibits reduced effector function such as, for example, C1q binding, complement dependent cytotoxicity (CDC), Fc receptor binding, antibody-dependent cell-mediated cytotoxicity (ADCC), phagocytosis, opsonization, and transcytosis. In one embodiment, the anti-CB1 antibody or antigen-binding fragment thereof binds CB1 and comprises one or more Fc region modifications that reduce, impair, or eliminate one or more effector functions. For example, in one embodiment, the anti-CB1 antibodies and antigen-binding fragments thereof disclosed herein bind CB1 but exhibit reduced, impaired, or absent C1q binding and/or CDC and/or ADCC. Fc modifications may be amino acid insertions, deletions, or substitutions, or may be chemical modifications. For example, Fc region modifications may be made to increase or decrease complement binding, to increase or decrease ADCC or CDC, or to modify glycosylation. Various Fc modifications are known in the art and have been described, for example, in Labrijin et al. (2009) Nature Biotech. 27(8):767-771; Greenwood et al. (1993) Eur. J. Immunol. 23:1098-1104; Mueller et al. (1997) Mol. Immunol. 34:441-452; and Rother et al. (2007) Nature Biotechnol. 25: 1256-1264. Any of the Fc modifications known in the art may be applied to the exemplary CB1 antibodies disclosed herein to alter effector function. In an embodiment, the anti-CB1 antibody or antigen-binding fragment thereof has certain mutations, e.g., L234A/L235A (“LALA”), S228P, A330S, P331S, E233P/L234V/L235A, A327G/A330S/P331S, L234F/L235E/P331S, and N297Q.

The binding proteins provided herein may be produced by any of a number of techniques known in the art. For example, expression from host cells, wherein expression vector(s) encoding the CB1 binding proteins are transfected into a host cell by standard techniques. Although it is possible to express the CB1 binding proteins provided herein in either prokaryotic or eukaryotic host cells, mammalian host cells are more likely than prokaryotic cells to assemble and secrete a properly folded and immunologically active binding protein.

In an exemplary system for recombinant expression of CB1 binding proteins is a recombinant expression vector encoding both the CB1 antibody heavy chain and the light chain is introduced into dhfr-CHO cells by calcium phosphate-mediated transfection. Within the recombinant expression vector, the CB1 antibody heavy and light chain sequences are each operatively linked to CMV enhancer and promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the CB1 antibody heavy and light chains and intact CB1 antibody protein is recovered from the culture medium. Standard molecular biology techniques are used to prepare the recombinant expression vector, transfect the host cells, select for transformants, culture the host cells and recover the CB1 antibody protein from the culture medium.

### Bioequivalents

The anti-CB1 antibodies and antibody fragments of the present disclosure encompass proteins having amino acid sequences that vary from those of the described antibodies but that retain the ability to bind human CBT. Such variant antibodies and antibody fragments comprise one or more additions, deletions, or substitutions of amino acids when compared to parent sequence, but exhibit biological activity that is essentially equivalent to that of the described antibodies. Likewise, the anti-CB1 antibody-encoding DNA sequences of the present invention encompass sequences that comprise one or more additions, deletions, or substitutions of nucleotides when compared to the disclosed sequence, but that encode an anti-CB1 antibody or antibody fragment that is essentially bioequivalent to an anti-CBT antibody or antibody fragment of the invention. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency. In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching. In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or one or more of its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-CBT antibodies may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-CB1 antibody variants comprising amino acid changes which modify the glycosylation characteristics of the antibodies, e.g., mutations that eliminate or remove glycosylation.

### Species Selectivity and Species Cross-Reactivity

The present invention also includes anti-CB1 antibodies that bind to human CBT and to CBT from one or more non-human species. For example, the anti-CB1 antibodies of the invention may bind to human CBT and may bind to one or more of mouse, rat, guinea pig, hamster, gerbil, pig, cat, dog, rabbit, goat, sheep, cow, horse, camel, cynomolgus monkey, marmoset, rhesus or chimpanzee CBT. According to certain embodiments of the invention, the anti-CB1 antibodies bind to human CBT but not to CBT from other species.

### Immunoconjugates

The invention encompasses anti-CB1 antibodies conjugated to a therapeutic moiety (“immunoconjugate”), such as a cytotoxin, a chemotherapeutic drug, an immunosuppressant or a radioisotope. Cytotoxic agents include any agent that is detrimental to cells. Examples of suitable cytotoxic agents and chemotherapeutic agents for forming immunoconjugates are known in the art and described herein.

### Multispecific Binding Proteins

The antibodies of the present invention may be monospecific, bi-specific, or multispecific. Multispecific antibodies may be specific for different epitopes of one target polypeptide or may contain antigen-binding domains specific for more than one target polypeptide. See, e.g., Tutt et al. (1991) J. Immunol. 147:60-69; Kufer et al. (2004) Trends Biotechnol. 22:238-244. The anti-CB1 antibodies of the present invention can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. For example, the present invention includes bi-specific antibodies wherein one arm of an immunoglobulin is specific for human CB1 or a fragment thereof, and the other arm of the immunoglobulin is specific for a second therapeutic target or is conjugated to a therapeutic moiety.

### Use of Binding Proteins in Various Diseases

The antibodies and binding proteins of the invention are useful for the treatment, prevention and/or amelioration of any disease or disorder associated with or mediated by CB1 expression or activity, or treatable by blocking the interaction between CB1 and a CB1 ligand (e.g., a cannabinoid) or otherwise inhibiting CB1 activity and/or signaling, and/or promoting receptor internalization and/or decreasing cell surface receptor number. The term “a disorder in which CB1 activity is detrimental” means a disorder or disease in which the presence or activity (e.g., aberrant or over-activity) of CB1 in a subject suffering from the disorder is either responsible for the pathophysiology of the disorder or disease or a factor that contributes to a worsening of the disorder or disease. Accordingly, a disorder in which CB1 activity is detrimental is a disorder in which reduction of CB1 activity is expected to alleviate the symptoms and/or progression of the disorder.

Binding protein molecules provided herein are useful as therapeutic molecules to treat various diseases or conditions, e.g., wherein CB1 proteins are detrimental. For example, the binding molecules provided herein include any disease or condition characterized by the overexpression, upregulation, or increased activity or signaling of CB1 or a failure of healthy homeostatic regulatory mechanisms that may result therein. Such diseases and conditions include obesity, syndromic obesities including Prader-Willi syndrome, Alström syndrome, Bardet-Biedel syndrome (BBS), Albright Hereditary Osteodystrophy (AHO), and SIM1 deletion syndrome; diabetes and related complications; dyslipidemia; liver diseases such as, for example, non-alcoholic steatohepatitis (NASH), non-alcoholic fatty liver disease, and primary biliary cirrhosis; fibrosis, for example, kidney fibrosis; chronic kidney disease; renal disease; metabolic diseases, osteoporosis, atherosclerosis, inflammatory disease, cardiovascular disease, cancer, pain, systemic sclerosis, multiple sclerosis spasticity, glaucoma, and nicotine addiction.

### Pharmaceutical Compositions

The invention provides pharmaceutical compositions comprising the anti-CB1 binding protein, e.g., antibodies or antigen-binding fragments thereof. The pharmaceutical compositions of the invention are formulated with suitable excipients, carriers, prophylactic agents, therapeutics agents, and other agents that improved the stability, delivery, tolerance, and effectiveness of the anti-CB1 binding protein. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. (1998) J. Pharm. Sci. Technol. 52: 238-311.

The pharmaceutical compositions comprising CB1 binding proteins provided herein are for use in, but not limited to, diagnosing, detecting, or monitoring a disorder, in preventing, treating, managing, or ameliorating a disorder or one or more symptoms thereof, and/or in research.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Methods of administering a prophylactic or therapeutic agent provided herein include, but are not limited to, parenteral administration (e.g., intradermal, intramuscular, intraperitoneal, intravenous and subcutaneous), epidural administration, intratumoral administration, mucosal administration (e.g., intranasal and oral routes) and pulmonary administration (e.g., aerosolized compounds administered with an inhaler or nebulizer). The formulation of pharmaceutical compositions for specific routes of administration, and the materials and techniques necessary for the various methods of administration are available and known to one skilled in the art.

Dosage regimens may be adjusted to provide the optimum desired response (e.g., a therapeutic or prophylactic response). For example, a single bolus may be administered, several divided doses may be administered overtime or the dose may be proportionally reduced or increased as indicated by the exigencies of the therapeutic situation. It is especially advantageous to formulate parenteral compositions in dosage unit form for ease of administration and uniformity of dosage. The term “dosage unit form” refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms provided herein are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, CH), HUMALOG MIX 75/25™ pen, HUMALOG™ pen, HUMALIN70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany). Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (AbbVie, Inc., Abbott Park, IL)

In certain situations, the pharmaceutical composition can be delivered in a controlled release system. In one embodiment, a pump may be used (Sefton (1987) CRC Crit. Ref. Biomed. Eng. 14: 201-240). In another embodiment, polymeric materials can be used (Medical Applications of Controlled Release, Langer and Wise (eds.), 1974, CRC Pres., Boca Raton, FL). In another embodiment, a controlled release system can be placed in proximity of the composition's target, thus requiring only a fraction of the systemic dose (Goodson (1984) in Medical Applications of Controlled Release, supra, 2: 115-138). Other controlled release systems are discussed in the review by Langer (1990) Science 249: 1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

The dose of antibody administered to a patient may vary depending upon the age and the size of the patient, target disease, disease stage, gender, presence of medical complications, other medication, conditions, route of administration, and the like. The preferred dose is typically calculated according to body weight or body surface area. When an antibody of the present invention is used for treating a condition or disease associated with CB1 activity in an adult patient, it may be advantageous to intravenously administer the antibody of the present invention normally at a single dose of about 0.01 to about 100 mg/kg body weight. Depending on the severity of the condition, the frequency and the duration of the treatment can be adjusted. Effective dosages and schedules for administering anti-CB1 antibodies may be determined empirically; for example, patient progress can be monitored by periodic assessment, and the dose adjusted accordingly. Moreover, interspecies scaling of dosages can be performed using well-known methods in the art (e.g., Mordenti et al. (1991) Pharmaceut. Res. 8: 1351-1359). It is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed composition.

### Combination Therapy

A binding protein provided herein also can also be administered with one or more additional therapeutic agents useful in the treatment of various diseases, the additional agent being selected by the skilled artisan for its intended purpose. For example, the additional agent can be a therapeutic agent art-recognized as being useful to treat the disease or condition being treated by the CB1 binding proteins provided herein. The combination can also include more than one additional agent.

Non-limiting examples of such additional therapeutically active components include other CB1 antagonists (e.g., a second anti-CB1 antibody or small molecule inhibitor of CB1 (e.g., rimonabant, taranabant, AM251, AM1387, AM4113, cannabigerol, ibipinabant, otenabant, surinabant, tetrahydrocannabivarin, and virodhamine, and AM6545), an antagonist of another CB1 family member.

The present invention also includes therapeutic combinations comprising any of the anti-CB1 antibodies mentioned herein and an additional inhibitor, wherein the inhibitor is an aptamer, an antisense molecule, a ribozyme, an siRNA, a peptibody, a nanobody or an antibody fragment (e.g., Fab fragment; F(ab′)₂ fragment; Fd fragment; Fv fragment; scFv; dAb fragment; or other engineered molecules, such as diabodies, triabodies, tetrabodies, minibodies and minimal recognition units). The anti-CB1 antibodies of the invention may also be administered and/or co-formulated in combination with additional therapeutic agents. The additional therapeutically active component(s) may be administered just prior to, concurrent with, or shortly after the administration of an anti-CB1 antibody of the present invention; (for purposes of the present disclosure, such administration regimens are considered the administration of an anti-CB1 antibody “in combination with” an additional therapeutically active component). The present invention includes pharmaceutical compositions in which an anti-CB1 antibody of the present invention is co-formulated with one or more of the additional therapeutically active component(s) as described elsewhere herein.

The present invention also includes compositions and methods comprising a combination of an “antagonist antibody” and an “inverse agonist antibody.” An “antagonist anti-CB1 antibody” means an anti-CBT antibody that inhibits, diminishes or prevents the signaling activity of a ligand (e.g., a cannabinoid) for CBT. Non-limiting examples of antagonist antibodies of the present invention are M1, M2, M3, M4, M5, M6, M7, M8, M7-H1, M7-H2, M7-H3, M7-H4, M7-H5, M7-H6, M7-H7, M7-H8, M7-H9, M7-H10, M7-H11, M7-H12, M7-H13, M7-H14, M7-H15, M7-H16, M5-H1, M5-H2. An “inverse agonist anti-CBT antibody” means an anti-CBT antibody that causes induces a pharmacological response opposite of an agonist. Where an agonist increases the activity of a receptor above its basal level, whereas an inverse agonist decreases the activity below the basal level. Non-limiting examples of inverse agonist antibodies of the present invention include M7. The present inventors have conceived of combining an antagonist antibody and an inverse agonist antibody in order to synergistically or otherwise improve efficacy. Accordingly, the present invention includes pharmaceutical compositions comprising at least one antagonist antibody and at least one inverse agonist antibody. The present invention also includes therapeutic methods comprising administering to a subject a combination of an antagonist antibody and an inverse agonist antibody (either as separate administrations or as co-formulations).

Combination therapy agents include, but arenot limited to, antineoplastic agents, radiotherapy, chemotherapy such as DNA alkylating agents, cisplatin, carboplatin, anti-tubulin agents, paclitaxel, docetaxel, taxol, doxorubicin, gemcitabine, gemzar, anthracyclines, adriamycin, topoisomerase I inhibitors, topoisomerase II inhibitors, 5-fluorouracil (5-FU), leucovorin, irinotecan, receptor tyrosine kinase inhibitors (e.g., erlotinib, gefitinib), COX-2 inhibitors (e.g., celecoxlb), kinase inhibitors, and siRNAs.

### Diagnostics

The disclosure herein provides diagnostic applications including, but not limited to, diagnostic assay methods, diagnostic kits containing one or more CB1 binding proteins, and adaptation of the methods and kits for use in automated and/or semi-automated systems. The methods, kits, and adaptations provided may be employed in the detection, monitoring, and/or treatment of a disease or disorder in an individual.

The anti-CB1 antibodies of the present invention may also be used to detect and/or measure CB1, or CB1-expressing cells in a sample, e.g., for diagnostic purposes. For example, an anti-CB1 antibody, or fragment thereof, may be used to diagnose a condition or disease characterized by aberrant expression (e.g., over-expression, under-expression, lack of expression, etc.) of CB1. Exemplary diagnostic assays for CB1 may comprise, e.g., contacting a sample, obtained from a patient, with an anti-CB1 antibody of the invention, wherein the anti-CB1 antibody is labeled with a detectable label or reporter molecule. Alternatively, an unlabeled anti-CB1 antibody can be used in diagnostic applications in combination with a secondary antibody which is itself detectably labeled. Suitable detectable substances include various enzymes, prosthetic groups, fluorescent materials, chemiluminescent materials and radioactive materials. Examples of suitable enzymes include horseradish peroxidase, alkaline phosphatase, β-galactosidase, luciferase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, and phycoerythrin.

An example of a luminescent material is luminol and examples of suitable radioactive materials include (e.g., ³H, ¹⁴C, ³²P ³S, ⁹⁰Y, ⁹⁹Tc, ¹¹¹In, ¹²⁵I, ¹³¹j ¹⁷⁷Lu, ¹⁶⁶Ho, and ¹⁵³Sm.

lmmunoassays provided by the present disclosure may include sandwich immunoassays, radioimmunoassay (RIA), enzyme immunoassay (EIA), enzyme-linked immunosorbent assay (ELISA), competitive-inhibition immunoassays, fluorescence polarization immunoassay (FPIA), enzyme multiplied immunoassay technique (EMIT), bioluminescence resonance energy transfer (BRET), fluorescence activated cell sorting (FACS), and homogenous chemiluminescent assays, among others.

A chemiluminescent microparticle immunoassay may be used, which may use the ARCHITECT® automated analyzer (Abbott Laboratories, Abbott Park, IL).

Methods employing mass spectrometry are provided by the present disclosure and include, but are not limited to MALDI (matrix-assisted laser desorption/ionization) or by SELDI (surface-enhanced laser desorption/ionization).

Methods for collecting, handling, processing, and analyzing biological test samples using immunoassays and mass spectrometry are well-known to one skilled in the art.

### Kits

A kit for assaying a test sample for the presence, amount or concentration of an analyte, or fragment thereof, in a test sample is also provided. The kit comprises at least one component for assaying the test sample for the analyte, or fragment thereof, and instructions for assaying the test sample for the analyte, or fragment thereof. The at least one component for assaying the test sample for the analyte, or fragment thereof, can include a composition comprising a binding protein, as disclosed herein, and/or an anti-analyte binding protein (or a fragment, a variant, or a fragment of a variant thereof), which is optionally immobilized on a solid phase.

Optionally, the kit may comprise a calibrator or control, which may comprise isolated or purified analyte. The kit can comprise at least one component for assaying the test sample for an analyte by immunoassay and/or mass spectrometry. The kit components, including the analyte, binding protein, and/or anti-analyte binding protein, or fragments thereof, may be optionally labeled using any art-known detectable label. The materials and methods for the creation provided for in the practice of the present disclosure would be known to one skilled in the art.

The kit (or components thereof), as well as the method of determining the presence, amount or concentration of an analyte in a test sample by an assay, such as an immunoassay as described herein, can be adapted for use in a variety of automated and semi-automated systems (including those wherein the solid phase comprises a microparticle), as described, for example, in U.S. Pat. Nos. 5,089,424 and 5,006,309, and as commercially marketed, for example, by Abbott Laboratories (Abbott Park, IL) as ARCHITECT®. Other platforms available from Abbott Laboratories include, but are not limited to, AxSYM®, IMx® (see, for example, U.S. Pat. No. 5,294,404, PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. lmmunosensors and their methods of manufacture and operation in single-use test devices are described, for example in. U.S. Pat. Nos. 5,063,081, 7,419,821, 7,682,833, 7,723,099, and 9.035,027; and US Publication Nos. 20040018577 and 20060160164.

It will be readily apparent to those skilled in the art that other suitable modifications and adaptations of the methods described herein are obvious and may be made using suitable equivalents without departing from the scope of the embodiments disclosed herein. Having now described certain embodiments in detail, practice of the invention will be more fully understood from the following examples, which are presented herein for illustration only and should not be construed as limiting the invention in any way.

### Examples

### Example 1: Generation and Selection of Anti-CB1 Antibodies for Functional Evaluation

CD2F1 mice were immunized with human CB-1 antigen in the presence or absence of CB-1 antagonist JD5037 (Cayman Chemical, Ann Arbor, MI; Cat. No. 1205), to potentially stabilize the protein such that it presents itself in an adequate conformation during immunization. Mice were immunized in both hocks with approximately 5μg/30μl/hock of antigen with Titer Max Gold adjuvant (St. Loui, MO; Sigma Aldrich; Cat. No. T2684). Subsequently, mice were immunized twice a week with CpG (InvivoGen, San Diego, CA; Cat. No. ODN1826) and Alhydrogel (InvivoGen, San Diego, CA; Cat. No. vac-alu-250) for approximately 30 days. Serum was harvested on days 13 and 26 to determine antibody titer and its increase over time. Mice were euthanased on day 30 and popliteal and inguinal lymph nodes were collected for fusion and washed in Media B (a 1:1 mix of RPMI 1640 (Thermo Fisher Scientific-Gibco, San Diego, CA; Cat No. 11879020) and IMDM (Lonza, Anaheim, CA; Cat. No. 12-722F) with no nutrients added) and single cell suspensions were prepared. P3Ag8.563 myeloma cells (ATCC, Manasas, VA; Cat. No. PTA-9393) were harvested from culture and washed in Media B. The lymphocytes and the myeloma cells were mixed at a ratio of 1:1 and fused using an electrofusion BTX Harvard apparatus ECM 2001(BTX Harvard Apparatus, Holliston, MA; Cat. No. 45-0012). Fused cells were resuspended in recovery Media C (Stem Cell Technologies, Seattle, WA; Cat. No. 03803) and allowed to recover in a T75 cm² flask overnight at 37° C. The following day the fused cells were harvested and resuspended in hybridoma selection methycellulose Media D (Stem Cell Technologies, Seattle, WA; Cat. No. 03804) containing anti-mouse IgG FITC Clone Detect (Molecular Devices, San Jose, CA; Cat No. K8220). The cells were mixed and plated at 1×10⁶ per 10 mL of Media D. The plated cells were incubated at 37° C. for 7 days. Hybridoma colonies were picked and transferred to 96-well tissue culture plates containing hybridoma growth Media E (Stem Cell Technologies, Seattle, WA; Cat. No. 03805) using Clone Pix2 (Molecular Devices, San Jose, CA) based on the size of the colony along with its ability to display a strong FITC halo that was indicative of IgG production. When macroscopic colonies were observed, the supernatants were screened for cell binding using CHO parental and CHO-huCB-1 overexpressing cells. For this primary screen, hybridoma parental CHO cells were labeled with carboxyfluorescein succinimidyl ester (CFSE) (Invitrogen, Anaheim, CA; Cat. No. 34554) and mixed with non-CFSE labeled CHO-huCB1 overexpressing cells to allow for efficient and simultaneous screening on both cells lines and to identify huCB-1 specific binders. The clones that specifically bound CHO-huCB-1 overexpressing cells and did not bind the parental CHO cells were selected and moved forward for a confirmatory screen. A total of 97 clones were selected to move forward for small scale purification. The summary of the relevant fusions and primary screening is shown in Table 2.

TABLE 2
Cell Fusions and Primary Screening

| | Campaign 9 | Campaign 10 |
|---|---|---|
| | huCB1 antigen | huCB1 antigen + JD5037 |
| | Hock Immunization | Hock Immunizations |
| No. of Mice | 7 | 7 |
| Lymphocytes | 100 × 10⁶ | 100 × 10⁶ |
| Fused Cells | 30 × 10⁶ | 36 × 10⁶ |
| Hybridomas Screened | ~2400 | ~2880 |
| Primary huCB1- | 91 | 83 |
| CHO Binders | | |
| Confirmed huCB1- | 47 | 50 |
| CHO Binders | | |
| moCB1-CHO Cross | 14 | 16 |
| Reactive Clones | | |

### Example 2: Purification of Murine Anti-huCB-1 Specific Binding Clones

The hybridoma clones that were selected based on the confirmatory primary screen were further expanded in 50 mL low Ig media (1:1 IMDM (Lonza, Anaheim, CA; Cat. No. 12-722F): Ham's F12-K (Gibco, Anheim, CA; Cat. No. 21127022) media with 5% low Ig serum (Invitrogen, Grand Island, NY; Cat. No. 1625007), containing 5 mL of a 100 mM sodium pyruvate solution (Invitrogen, Grand Island, NY; Cat. No. 11360070), 5 ml of 100 mM of non-essential amino acids (Invitrogen, Grand Island, NY; Cat. No. 11140050) and 5 mL of 100 mM glutamine (Invitrogen, Grand Island, NY; Cat. No. 35050061)) in a T75 cm² flask for 3-4 weeks. Supernatants were harvested and purified using standard Protein A purification methods.

### Example 3: Functional Characterization of Mouse Anti-CB1 Antibodies in a cAMP Assay

The isolated mouse anti-huCB-1 antibodies were evaluated for their antagonist activity in a cAMP assay. cAMP assays were performed using a cAMP Hunter™ CHO-K1 CNR1 Gi Cell Line (DiscoverX/Eurofins, Fremont, CA; Cat. No. 95-0071C2), which overexpresses naturally Gi coupled, wildtype G-protein coupled receptors (GPCRs) and are designed to detect increases in intracellular cAMP levels in response to agonist stimulation of the receptor. cAMP Hunter™ CHO-K1 CNR1 Gi cells were treated with CB1 antibodies, an isotype control, or the small molecule CB1 antagonist JD5037, followed by an agonist challenge with 30 nM CP-55,940 (indicated as “Plus CP”) in the presence of forskolin. Antagonists were also tested without the addition of CP-55,940, to establish whether they had agonistic activity themselves. Forskolin activates the enzyme adenylate cyclase and increases intracellular levels of cAMP. For a Gi receptor, agonist binding inhibits intracellular cAMP accumulation induced by forskolin. Thus, in order to measure Gi-coupled receptors, the agonist compound CP-55,940 was added in the presence of forskolin. Activation of the Gi-coupled receptor therefore inhibits the forskolin-induced production of cAMP and, as a result, the dose response curve generated in the presence of agonist plus forskolin will have a negative slope. Briefly, cells were seeded in Cell Plating 2 Medium (DiscoverX/Eurofins; Fremont, CA; Cat No. 93-0563R2A) at 1.5×10⁴ cells/well in 96-well plates (Costar, Fisher Scientific, San Diego, CA; Cat. No. 3909) and incubated overnight at 37° C., 5% CO₂. The following day, the culture medium was replaced with 30 μl of Cell Assay Buffer (CAB; 1x HBSS/10 nM HEPES (ThermoFisher, Anaheim, CA; Cat. Nos. 14025134 and 15630080, respectively) and treated with test antibodies or an isotype control (7.5 μl of a 6× concentrated working dilution). Plates were incubated for 30 minutes at 37° C., 5% CO₂. 7.5 μl of agonist challenge (0.18 μM CP 55,940 in CAB containing 90 μM forskolin) was added to each well and plates were incubated for another 30 minutes at 37° C., 5% CO₂. Plates were processed for cAMP reading using the HitHunter® cAMP Assay Detection Kit for Biologics (DiscoverX/Eurofins; Fremont, CA; Cat. No. 90-0075LM25) following the manufacturer's instructions. The initial assessment of the isolated clones was done at a single concentration of 30 μg/mL.

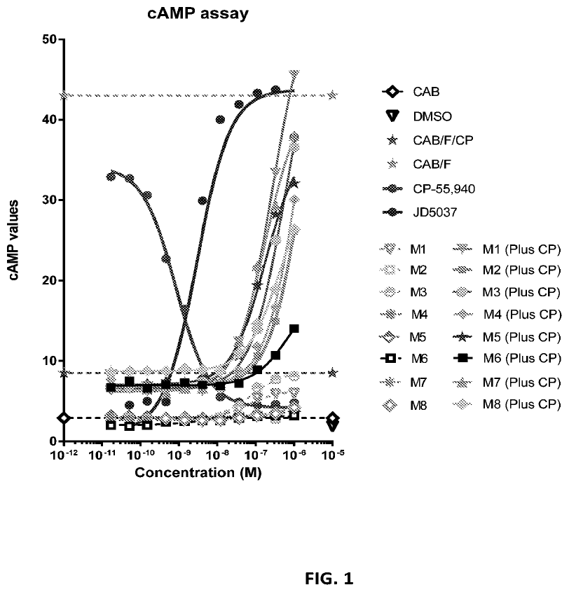

Of the 112 clones tested, only eight clones displayed antagonistic activity. Two clones showed some degree of antagonism in the absence of the agonist, M3 and to a lesser extent M1. The eight antibodies M1, M2, M3, M4, M5, M6, M7, and M8 were further evaluated by titrating the concentration of the Abs to obtain dose response curves and actual EC50 values (Table 3 and FIG. 1).

### Example 4: Functional Characterization of Mouse Anti-CB1 Antibodies in a pERK Assay

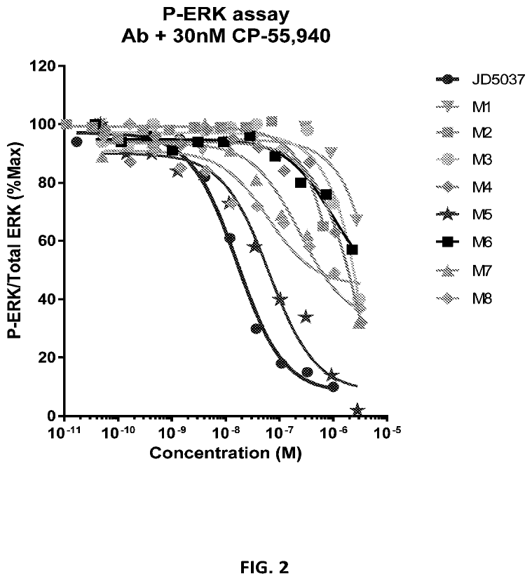

The eight antibodies that were functional antagonists in the cAMP assay were further evaluated in a pERK phosphorylation assay performed using the cAMP Hunter™ CHO-K1 CNR1 Gi Cell Line (DiscoverX/Eurofins, Fremont, CA; Cat. No. 95-0071C2). Briefly, cells were seeded into 96-well plates at 2×10⁴ cells/well in Assay Complete Cell Culture Medium from Kit-107 (DiscoverX/Eurofins, Fremont, CA; Cat. No. 92-3107G) plus 800 μg/mL G418 and incubated at 37° C., 5% C02. The following day, the culture medium was replaced with 100 μl/well of serum-free F-12K starvation medium (Invitrogen, Grand Island, NY; Cat. No. 11765054). Plates were incubated for a further day at 37° C., 5% C02. On the day of treatment, the F-12K medium was replaced with 30 μl/well of fresh F-12K medium. A test antibody or isotype control (7.5 μl of a 6× concentrated working dilution) was then added to the wells and the plates were incubated for 10 minutes at 37° C., 5% C02. 7.5 μl of agonist (a 6-fold working solution comprising 0.18 μM CP 55,940 in CAB with 90 μM forskolin) was added to each well and the plates incubated for another 10 minutes at 37° C., 5% C02. Plates were processed for p-ERK/Total ERK using a Meso Scale Discovery (MSD) kit (Meso Scale Discovery, Rockville, Maryland; Cat. No. K15107D) following the manufacturer's instructions (Table 3 and FIG. 2).

TABLE 3
Summary of Mouse Anti-huCB-1 Antibody Cellular Assay Data

| | cAMP | | | | P-ERK | | |
|---|---|---|---|---|---|---|---|
| | Mean | EC50 (nM) | | | Mean | IC50 (nM) | |
| CB1 | cAMP | Data | Data | Data | P-ERK | Data | Data |
| Antibody | (% Amgen) | Set 1 | Set 2 | Set 3 | (% inhibition) | Set 1 | Set 2 |
| M1 | 75 | 43 | 170 | 150 | 68 | 163 | 165 |
| M2 | 49 | 380 | 340 | 270 | 59 | 219 | 211 |
| M3 | 71 | 290 | 290 | 330 | 59 | 296 | 534 |
| M4 | 41 | 180 | 320 | 430 | 70 | 97 | 890 |
| M5 | 61 | 91 | 170 | 170 | 101 | 191 | 50 |
| M6 | 21 | 310 | 290 | 640 | 42 | 144 | 300 |
| M7 | 61 | 78 | 120 | 120 | 95 | 117 | 170 |
| M8 | 29 | 96 | 190 | 370 | 64 | 36 | 47 |

### Example 5: EC50 Binding of Mouse Anti-CB-1 Antibodies to CB-1 CHO Overexpressing Cells

Antibody binding was tested in a fluorescence activated cell sorting (FACS)-based assay for the ability of the mouse anti-CB1 antibodies to bind parental CHO cells, human CB-1 overexpressing CHO cells and mouse CB-1 overexpressing CHO cells to obtain binding curves and EC50 values. The three cell lines were harvested, washed and dispensed at 1×10⁵ cells per well of a v-bottomed 96-well polycarbonate FACS plates (Corning, Coming, NY, Cat. No. 3357) in 50 μl of FACS buffer (1×PBS/2 mM EDTA and 1% FBS (ThermoFisher Scientific, Anaheim, CA; Cat. No. 10438-026). Serial dilutions of the antibodies were prepared at 2X concentrations starting at 200 nM and serially diluted 3-fold.

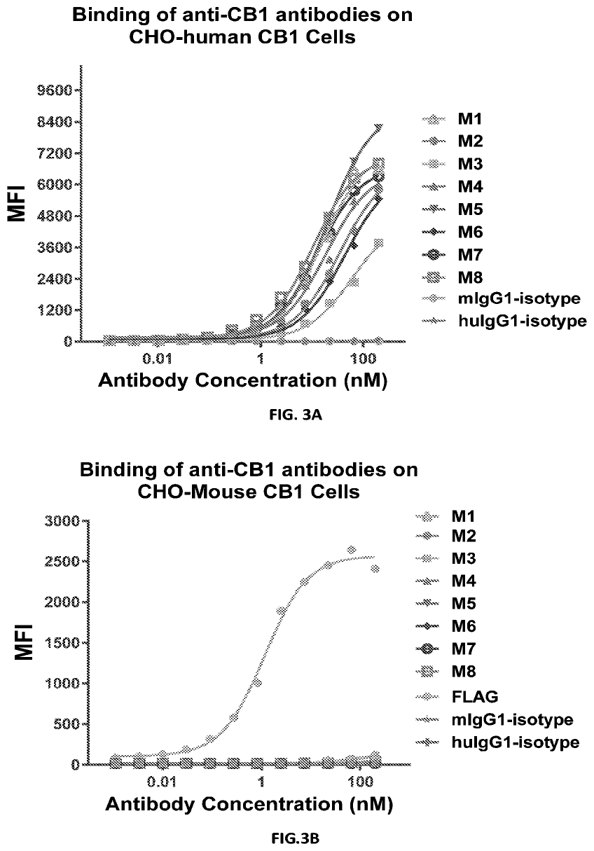

The titrated antibodies were added to the plates containing the three different cell lines (parental, human and mouse CB-1 CHO cells) and incubated at 4° C. for 1 hour. The plates were washed 3X with FACS buffer. The cells were resuspended in 50 μl of 1/5,000 dilution of goat anti-mouse IgG-HRP (Jackson Immuno Research, West Grove, PA; Cat. No. 115-035-003), incubated at 4° C. for 30 minutes, washed 3×with FACS buffer, and data collected on the BD FACS Canto (BD Biosciences, San Jose, CA; Cat. No. 338962) and analyzed using FlowJo (FlowJo LLC, Ashland, OR) (Table 4). None of the eight functional antibodies tested were mouse cross reactive (FIG. 3).

TABLE 4
Cell Binding Affinity Mouse Clones
for CB1 Over-Expressing Cells

| Clone | M1 | M2 | M3 | M4 | M5 | M6 | M7 | M8 |
|---|---|---|---|---|---|---|---|---|
| EC50 | 15.7 | 34.72 | 61.62 | 19.19 | 20.16 | 43.52 | 12.97 | 10.66 |
| (nM) | | | | | | | | |

### Example 6: EC50 Analysis of Functional Anti-CB-1 Antibodies and Evaluation for Conformational Binding

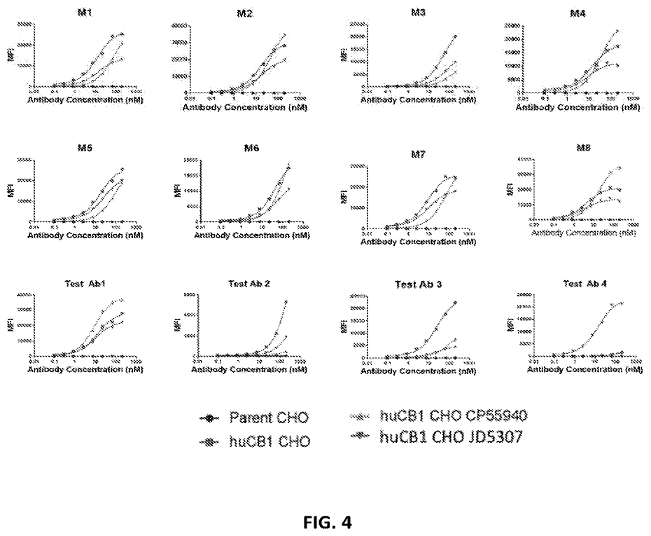

The goal of this experiment was to determine if anti-CB1 antagonist antibodies have differential binding capabilities for CB1 in neutral, antagonist, or agonist state confirmations. Four different cell line preparations were used: CHO-huCB1 (generated in-house), CHO-huCB1 pre-incubated with inverse agonist JD5037 (Cayman Chemicals, Ann Arbor, MI; Cat. No. 1392116-14-1), CHO-huCB1 pre-incubated with agonist CP-55,940 (TOCRIS, Minneapolis, MN; Cat. No. 0949), and parent CHO-S cells (ThermoFisher Scientific, VA; Cat. No. R80007). 2×10⁷ parental CHO-S and CHO-hu CB1 cells were set aside in FACS buffer. In addition, 2×10⁷ CHO-huCBlcells coated with inverse agonist JD5037 or agonist CP-55,940 were incubated at 4° C. for 1 hour. Post incubation these two coated cell lines were washed 2×in FACS buffer and resuspended at 2×10⁷ in FACS buffer containing the inverse agonist or agonist molecules, respectively. All four cell lines were plated in v bottom FACS plates (Coming, Coming, NY; Cat. No. 3357) and pre-titrated anti-CB1 test antibodies were added to the cells and evaluated for binding by BD FACS Canto (BD Biosciences, San Jose, CA; Cat. No. 338962). As shown in FIG. 4, the antibodies did not bind to the CHO parental cells (blue curve), the above eight functional Abs (M1, M2, M3, M4, M5, M6, M7, and M8) did not display a preferential binding in the presence of agonist or antagonist as displayed by binding observed under all conditions (red, purple and green curves). Only 2 test antibodies that were otherwise non-functional in the cAMP or pERK assays, displayed a preferential binding in the presence of antagonist and absence of agonist respectively. This suggests that a functional anti-CB1 antibody may not be associated with a binding conformation that is brought about in the presence of a known receptor agonist or antagonist.

### Example 7: Sequence Identification and Analysis of Mouse Anti-CB1 Antibodies

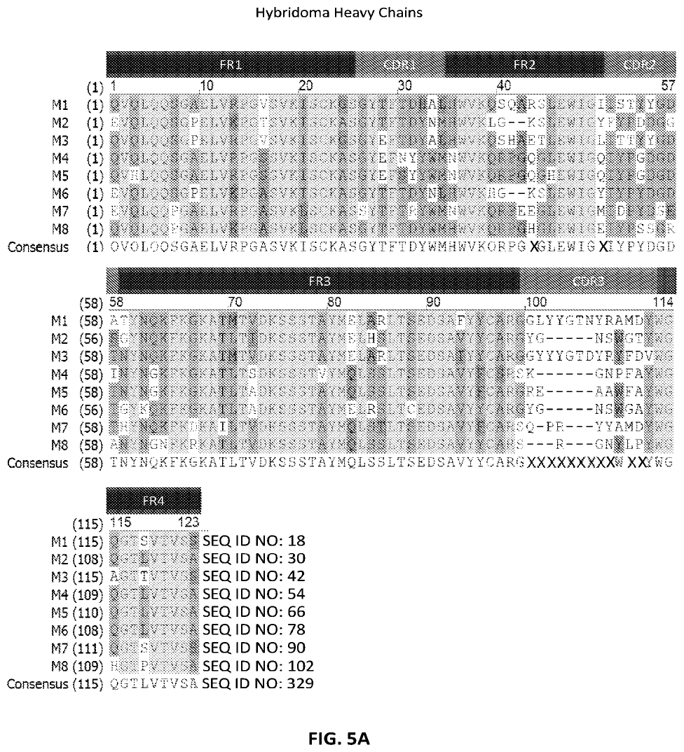

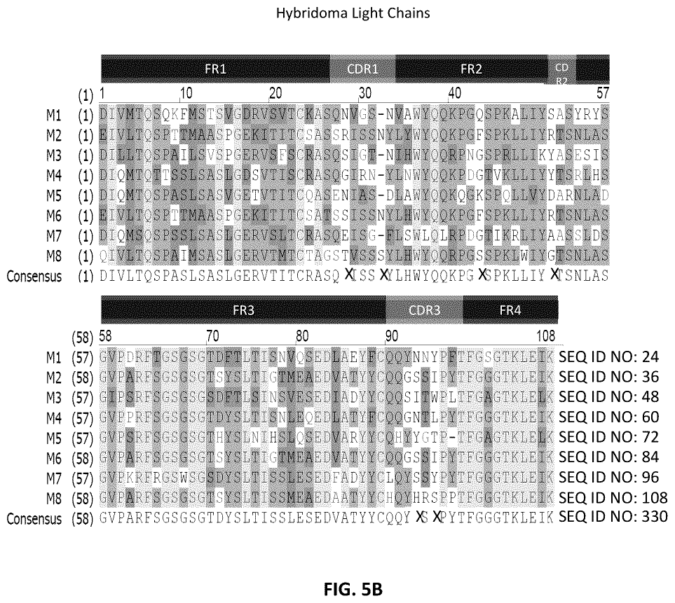

The hybridomas of the eight murine anti-huCB-1 antibodies were harvested as cell pellets and the supernatants were used to determine the isotype of each of the hybridomas using a standard mouse isotyping ELISA kit (Pierce/ThermoFisher Scientific, San Diego, CA; Cat. No. 37503). Four of the antibodies (M1, M3, M4, and M6) are IgG2a,K and four of the antibodies (M2, M5, M7, and M8) are IgG2b,K. The pellets were processed for RNA and cDNA and the SMARTER RACE Amplification kit (Clontech, Mountain View, CA; Cat. No. 634859) was used to process the cDNA for sequencing. The isotype of each of the antibodies was used to design the reverse primers for the constant region of the heavy chains and the light chain kappa constant region and SeqAmp polymerase (CloneTech, Mountain View, CA; Cat. No. 638504) as the forward primer. A MOPC21 PNA primer (synthesized based on sequence) was included to prevent amplification of the aberrant light chain that often appears during the sequencing process and can interfere with identification of the actual light chain variable region sequence. A total of 8 unique sequences and 7 unique families were identified. The sequences of the eight clones are provide in Table 5. A consensus sequence of the heavy and light chains of the hybridoma antibodies is provided in FIGS. 5A and 5B, respectively.

TABLE 5
Amino Acid Sequences of Mouse-Human Fc Chimeric CB1 Antibodies

| Code | Protein | Amino Acid Sequence | SEQ ID |
|---|---|---|---|
| Name | Region | 1234567890123456789012345678901234567890 | NO |
| M1 | HC | QVQLQQSGAELVRPGVSVKISCKGSGYTFTDHALHWVKQS | 17 |
| | | QARSLEWIGIISTYYGDATYNQKFKGKATMTVDKSSSTAY | |
| | | MELARLTSEDSAFYYCARGGLYYGTNYRAMDYWGQGTSVT | |
| | | VSSASTKGPSVFPLAPSSKSTSGGTAALGCLVKDYFPEPV | |
| | | TVSWNSGALTSGVHTFPAVLQSSGLYSLSSVVTVPSSSLG | |
| | | TQTYICNVNHKPSNTKVDKRVEPKSCDKTHTCPPCPAPEL | |
| | | LGGPSVFLFPPKPKDTLMISRTPEVTCVVVDVSHEDPEVK | |
| | | FNWYVDGVEVHNAKTKPREEQYNSTYRVVSVLTVLHQDWL | |
| | | NGKEYKCKVSNKALPAPIEKTISKAKGQPREPQVYTLPPS | |
| | | REEMTKNQVSLTCLVKGFYPSDIAVEWESNGQPENNYKTT | |
| | | PPVLDSDGSFFLYSKLTVDKSRWQQGNVFSCSVMHEALHN | |
| | | HYTQKSLSLSPGK | |
| | VH | QVQLQQSGAELVRPGVSVKISCKGSGYTFTDHALHWVKQS | 18 |
| | | QARSLEWIGIISTYYGDATYNQKFKGKATMTVDKSSSTAY | |
| | | MELARLTSEDSAFYYCARGGLYYGTNYRAMDYWGQGTSVT | |
| | | VSS | |
| | CH | ASTKGPSVFPLAPSSKSTSGGTAALGCLVKDYFPEPVTVS | 19 |
| | | WNSGALTSGVHTFPAVLQSSGLYSLSSVVTVPSSSLGTQT | |
| | | YICNVNHKPSNTKVDKRVEPKSCDKTHTCPPCPAPELLGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | GYTFTDHA | 20 |
| | CDR-H2 | IISTYYGDA | 21 |
| | CDR-H3 | ARGGLYYGTNYRAMDY | 22 |
| | LC | DIVMTQSQKFMSTSVGDRVSVTCKASQNVGSNVAWYQQKP | 23 |
| | | GQSPKALIYSASYRYSGVPDRFTGSGSGTDFTLTISNVQS | |
| | | EDLAEYFCQQYNNYPFTFGSGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIVMTQSQKFMSTSVGDRVSVTCKASQNVGSNVAWYQQKP | 24 |
| | | GQSPKALIYSASYRYSGVPDRFTGSGSGTDFTLTISNVQS | |
| | | EDLAEYFCQQYNNYPFTFGSGTKLEIK | |
| | CL | RTVAAPSVFIFPPSDEQLKSGTASVVCLLNNFYPREAKVQ | 25 |
| | | WKVDNALQSGNSQESVTEQDSKDSTYSLSSTLTLSKADYE | |
| | | KHKVYACEVTHQGLSSPVTKSFNRGEC | |
| | CDR-L1 | QNVGSN | 26 |
| | CDR-L2 | SAS | 27 |
| | CDR-L3 | QQYNNYPFT | 28 |
| M2 | HC | EVQLQQSGPELVKPGTSVKISCKASGYTFTDYNMHWVKLG | 29 |
| | | KSLEWIGYFYPDDGGSGYNQKFKGKATLTIDKSSSTAYME | |
| | | LHSLTSEDSAVYYCARGYGNSWGTYWGQGTLVTVSAASTK | |
| | | GPSVFPLAPSSKSTSGGTAALGCLVKDYFPEPVTVSWNSG | |
| | | ALTSGVHTFPAVLQSSGLYSLSSVVTVPSSSLGTQTYICN | |
| | | VNHKPSNTKVDKRVEPKSCDKTHTCPPCPAPELLGGPSVF | |
| | | LFPPKPKDTLMISRTPEVTCVVVDVSHEDPEVKFNWYVDG | |
| | | VEVHNAKTKPREEQYNSTYRVVSVLTVLHQDWLNGKEYKC | |
| | | KVSNKALPAPIEKTISKAKGQPREPQVYTLPPSREEMTKN | |
| | | QVSLTCLVKGFYPSDIAVEWESNGQPENNYKTTPPVLDSD | |
| | | GSFFLYSKLTVDKSRWQQGNVFSCSVMHEALHNHYTQKSL | |
| | | SLSPGK | |
| | VH | EVQLQQSGPELVKPGTSVKISCKASGYTFTDYNMHWVKLG | 30 |
| | | KSLEWIGYFYPDDGGSGYNQKFKGKATLTIDKSSSTAYME | |
| | | LHSLTSEDSAVYYCARGYGNSWGTYWGQGTLVTVSA | |
| | CH | ASTKGPSVFPLAPSSKSTSGGTAALGCLVKDYFPEPVTVS | 31 |
| | | WNSGALTSGVHTFPAVLQSSGLYSLSSVVTVPSSSLGTQT | |
| | | YICNVNHKPSNTKVDKRVEPKSCDKTHTCPPCPAPELLGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | GYTFTDYN | 32 |
| | CDR-H2 | FYPDDGGS | 33 |
| | CDR-H3 | ARGYGNSWGTY | 34 |
| | LC | EIVLTQSPTTMAASPGEKITITCSASSRISSNYLYWYQQK | 35 |
| | | PGFSPKLLIYRTSNLASGVPARFSGSGSGTSYSLTIGTME | |
| | | AEDVATYYCQQGSSIPYTFGGGTKLEIKRTVAAPSVFIFP | |
| | | PSDEQLKSGTASVVCLLNNFYPREAKVQWKVDNALQSGNS | |
| | | QESVTEQDSKDSTYSLSSTLTLSKADYEKHKVYACEVTHQ | |
| | | GLSSPVTKSFNRGEC | |
| | VL | EIVLTQSPTTMAASPGEKITITCSASSRISSNYLYWYQQK | 36 |
| | | PGFSPKLLIYRTSNLASGVPARFSGSGSGTSYSLTIGTME | |
| | | AEDVATYYCQQGSSIPYTFGGGTKLEIK | |
| | CL | RTVAAPSVFIFPPSDEQLKSGTASVVCLLNNFYPREAKVQ | 37 |
| | | WKVDNALQSGNSQESVTEQDSKDSTYSLSSTLTLSKADYE | |
| | | KHKVYACEVTHQGLSSPVTKSFNRGEC | |
| | CDR-L1 | SRISSNY | 38 |
| | CDR-L2 | RTS | 39 |
| | CDR-L3 | QQGSSIPYT | 40 |
| M3 | HC | QVQLQQSGPELVRPGVSVKISCKGSGYEFTDYALHWVKQS | 41 |
| | | HAETLEWIGLITTYYGDTNYNQKFKGKATMTVDKSSSTAY | |
| | | MELARLTSEDSAIYYCARGGYYYGTDYRYFDVWGAGTTVT | |
| | | VSSASTKGPSVFPLAPSSKSTSGGTAALGCLVKDYFPEPV | |
| | | TVSWNSGALTSGVHTFPAVLQSSGLYSLSSVVTVPSSSLG | |
| | | TQTYICNVNHKPSNTKVDKRVEPKSCDKTHTCPPCPAPEL | |
| | | LGGPSVFLFPPKPKDTLMISRTPEVTCVVVDVSHEDPEVK | |
| | | FNWYVDGVEVHNAKTKPREEQYNSTYRVVSVLTVLHQDWL | |
| | | NGKEYKCKVSNKALPAPIEKTISKAKGQPREPQVYTLPPS | |
| | | REEMTKNQVSLTCLVKGFYPSDIAVEWESNGQPENNYKTT | |
| | | PPVLDSDGSFFLYSKLTVDKSRWQQGNVFSCSVMHEALHN | |
| | | HYTQKSLSLSPGK | |
| | VH | QVQLQQSGPELVRPGVSVKISCKGSGYEFTDYALHWVKQS | 42 |
| | | HAETLEWIGLITTYYGDTNYNQKFKGKATMTVDKSSSTAY | |
| | | MELARLTSEDSAIYYCARGGYYYGTDYRYFDVWGAGTTVT | |
| | | VSS | |
| | CH | ASTKGPSVFPLAPSSKSTSGGTAALGCLVKDYFPEPVTVS | 43 |
| | | WNSGALTSGVHTFPAVLQSSGLYSLSSVVTVPSSSLGTQT | |
| | | YICNVNHKPSNTKVDKRVEPKSCDKTHTCPPCPAPELLGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | GYEFTDYA | 44 |
| | CDR-H2 | ITTYYGDT | 45 |
| | CDR-H3 | ARGGYYYGTDYRYFDV | 46 |
| | LC | DILLTQSPAILSVSPGERVSFSCRASQSIGTNIHWYQQRP | 47 |
| | | NGSPRLLIKYASESISGIPSRFSGSGSGSDFTLSINSVES | |
| | | EDIADYYCQQSITWPLTFGAGTKLELKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DILLTQSPAILSVSPGERVSFSCRASQSIGTNIHWYQQRP | 48 |
| | | NGSPRLLIKYASESISGIPSRFSGSGSGSDFTLSINSVES | |
| | | EDIADYYCQQSITWPLTFGAGTKLELK | |
| | CL | RTVAAPSVFIFPPSDEQLKSGTASVVCLLNNFYPREAKVQ | 49 |
| | | WKVDNALQSGNSQESVTEQDSKDSTYSLSSTLTLSKADYE | |
| | | KHKVYACEVTHQGLSSPVTKSFNRGEC | |
| | CDR-L1 | QSIGTN | 50 |
| | CDR-L2 | YAS | 51 |
| | CDR-L3 | QQSITWPLT | 52 |
| M4 | HC | QVQLQQSGAELVRPGSSVKISCKASGYEFNYYWMNWVKQR | 53 |
| | | PGQGLEWIGQIYPGDGDINYNGKFKGKATLTSDKSSSTVY | |
| | | MQLSSLTSEDSAVYFCSRSKGNPFAYWGQGTLVTVSAAST | |
| | | KGPSVFPLAPSSKSTSGGTAALGCLVKDYFPEPVTVSWNS | |
| | | GALTSGVHTFPAVLQSSGLYSLSSVVTVPSSSLGTQTYIC | |
| | | NVNHKPSNTKVDKRVEPKSCDKTHTCPPCPAPELLGGPSV | |
| | | FLFPPKPKDTLMISRTPEVTCVVVDVSHEDPEVKFNWYVD | |
| | | GVEVHNAKTKPREEQYNSTYRVVSVLTVLHQDWLNGKEYK | |
| | | CKVSNKALPAPIEKTISKAKGQPREPQVYTLPPSREEMTK | |
| | | NQVSLTCLVKGFYPSDIAVEWESNGQPENNYKTTPPVLDS | |
| | | DGSFFLYSKLTVDKSRWQQGNVFSCSVMHEALHNHYTQKS | |
| | | LSLSPGK | |
| | VH | QVQLQQSGAELVRPGSSVKISCKASGYEFNYYWMNWVKQR | 54 |
| | | PGQGLEWIGQIYPGDGDINYNGKFKGKATLTSDKSSSTVY | |
| | | MQLSSLTSEDSAVYFCSRSKGNPFAYWGQGTLVTVSA | |
| | CH | ASTKGPSVFPLAPSSKSTSGGTAALGCLVKDYFPEPVTVS | 55 |
| | | WNSGALTSGVHTFPAVLQSSGLYSLSSVVTVPSSSLGTQT | |
| | | YICNVNHKPSNTKVDKRVEPKSCDKTHTCPPCPAPELLGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | GYEFNYYW | 56 |
| | CDR-H2 | IYPGDGDI | 57 |
| | CDR-H3 | SRSKGNPFAY | 58 |
| | LC | DIQMTQTTSSLSASLGDSVTISCRASQGIRNYLNWYQQKP | 59 |
| | | DGTVKLLIYYTSRLHSGVPPRFSGSGSGTDYSLTISNLEQ | |
| | | EDLATYFCQQGNTLPYTFGGGTKLEIKRTVAAPSVEIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQTTSSLSASLGDSVTISCRASQGIRNYLNWYQQKP | 60 |
| | | DGTVKLLIYYTSRLHSGVPPRFSGSGSGTDYSLTISNLEQ | |
| | | EDLATYFCQQGNTLPYTFGGGTKLEIK | |
| | CL | RTVAAPSVFIFPPSDEQLKSGTASVVCLLNNFYPREAKVQ | 61 |
| | | WKVDNALQSGNSQESVTEQDSKDSTYSLSSTLTLSKADYE | |
| | | KHKVYACEVTHQGLSSPVTKSFNRGEC | |
| | CDR-L1 | QGIRNY | 62 |
| | CDR-L2 | YTS | 63 |
| | CDR-L3 | QQGNTLPYT | 64 |
| M5 | HC | QVHLQQSGAELVRPGSSVKISCKASGYEFSYYWMNWVKQR | 65 |
| | | PGQGHEWIGQIYPGDGDTNYNGKFKGKATLTADKSSSTAY | |
| | | MQLSSLTSEDSAVYFCARGREAAWFAYWGQGTLVTVSAAS | |
| | | TKGPSVFPLAPSSKSTSGGTAALGCLVKDYFPEPVTVSWN | |
| | | SGALTSGVHTFPAVLQSSGLYSLSSVVTVPSSSLGTQTYI | |
| | | CNVNHKPSNTKVDKRVEPKSCDKTHTCPPCPAPELLGGPS | |
| | | VFLFPPKPKDTLMISRTPEVTCVVVDVSHEDPEVKFNWYV | |
| | | DGVEVHNAKTKPREEQYNSTYRVVSVLTVLHQDWLNGKEY | |
| | | KCKVSNKALPAPIEKTISKAKGQPREPQVYTLPPSREEMT | |
| | | KNQVSLTCLVKGFYPSDIAVEWESNGQPENNYKTTPPVLD | |
| | | SDGSFFLYSKLTVDKSRWQQGNVFSCSVMHEALHNHYTQK | |
| | | SLSLSPGK | |
| | VH | QVHLQQSGAELVRPGSSVKISCKASGYEFSYYWMNWVKQR | 66 |
| | | PGQGHEWIGQIYPGDGDTNYNGKFKGKATLTADKSSSTAY | |
| | | MQLSSLTSEDSAVYFCARGREAAWFAYWGQGTLVTVSA | |
| | CH | ASTKGPSVFPLAPSSKSTSGGTAALGCLVKDYFPEPVTVS | 67 |
| | | WNSGALTSGVHTFPAVLQSSGLYSLSSVVTVPSSSLGTQT | |
| | | YICNVNHKPSNTKVDKRVEPKSCDKTHTCPPCPAPELLGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | GYEFSYYW | 68 |
| | CDR-H2 | IYPGDGDT | 69 |
| | CDR-H3 | ARGREAAWFAY | 70 |
| | LC | DIVLAQSPASLAVSLGQRATISCRASQSVSSFRYSYLHWY | |
| | | QQKPGQPPRLLIKYASNLESGVPARFSGSGSGTDFTLNIH | |
| | | PVEEEDTATYFCQHSWEIPFTFGSGTKLEIKRRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSFNRGEC | |
| | LC | DIQMTQSPASLSASVGETVTITCQASENIASDLAWYQQKQ | 71 |
| | | GKSPQLLVYDARNLADGVPSRFSGSGSGTHYSLNIHSLQS | |
| | | EDVARYYCQHYYGTPTFGAGTKLELKRTVAAPSVFIFPPS | |
| | | DEQLKSGTASVVCLLNNFYPREAKVQWKVDNALQSGNSQE | |
| | | SVTEQDSKDSTYSLSSTLTLSKADYEKHKVYACEVTHQGL | |
| | | SSPVTKSFNRGEC | |
| | VL | DIVLAQSPASLAVSLGQRATISCRASQSVSSFRYSYLHWY | |
| | | QQKPGQPPRLLIKYASNLESGVPARFSGSGSGTDFTLNIH | |
| | | PVEEEDTATYFCQHSWEIPFTFGSGTKLEIKR | |
| | VL | DIQMTQSPASLSASVGETVTITCQASENIASDLAWYQQKQ | 72 |
| | | GKSPQLLVYDARNLADGVPSRFSGSGSGTHYSLNIHSLQS | |
| | | EDVARYYCQHYYGTPTFGAGTKLELK | |
| | CL | RTVAAPSVFIFPPSDEQLKSGTASVVCLLNNFYPREAKVQ | 73 |
| | | WKVDNALQSGNSQESVTEQDSKDSTYSLSSTLTLSKADYE | |
| | | KHKVYACEVTHQGLSSPVTKSFNRGEC | |
| | CDR-L1 | QSVSSFRYSY | |
| | CDR-L1 | ENIASD | 74 |
| | CDR-L2 | YAS | |
| | CDR-L2 | DAR | 75 |
| | CDR-L3 | QHSWEIPFT | |
| | CDR-L3 | QHYYGTPT | 76 |
| M6 | HC | EVQLQQSGPELVKPGASVKISCKASGYTFTDYNLHWVKHG | 77 |
| | | KSLEWIGYIYPYDGDTGYKQKFKGKATLTADKSSSTAYME | |
| | | LRSLTCEDSAVYYCARGYGNSWGAYWGQGTLVTVSAASTK | |
| | | GPSVFPLAPSSKSTSGGTAALGCLVKDYFPEPVTVSWNSG | |
| | | ALTSGVHTFPAVLQSSGLYSLSSVVTVPSSSLGTQTYICN | |
| | | VNHKPSNTKVDKRVEPKSCDKTHTCPPCPAPELLGGPSVF | |
| | | LFPPKPKDTLMISRTPEVTCVVVDVSHEDPEVKFNWYVDG | |
| | | VEVHNAKTKPREEQYNSTYRVVSVLTVLHQDWLNGKEYKC | |
| | | KVSNKALPAPIEKTISKAKGQPREPQVYTLPPSREEMTKN | |
| | | QVSLTCLVKGFYPSDIAVEWESNGQPENNYKTTPPVLDSD | |
| | | GSFFLYSKLTVDKSRWQQGNVFSCSVMHEALHNHYTQKSL | |
| | | SLSPGK | |
| | VH | EVQLQQSGPELVKPGASVKISCKASGYTFTDYNLHWVKHG | 78 |
| | | KSLEWIGYIYPYDGDTGYKQKFKGKATLTADKSSSTAYME | |
| | | LRSLTCEDSAVYYCARGYGNSWGAYWGQGTLVTVSA | |
| | CH | ASTKGPSVFPLAPSSKSTSGGTAALGCLVKDYFPEPVTVS | 79 |
| | | WNSGALTSGVHTFPAVLQSSGLYSLSSVVTVPSSSLGTQT | |
| | | YICNVNHKPSNTKVDKRVEPKSCDKTHTCPPCPAPELLGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | GYTFTDYN | 80 |
| | CDR-H2 | IYPYDGDT | 81 |
| | CDR-H3 | ARGYGNSWGAY | 82 |
| | LC | EIVLTQSPTTMAASPGEKITITCSATSSISSNYLHWYQQK | 83 |
| | | PGFSPKLLIYRTSNLASGVPARFSGSGSGTSYSLTIGTME | |
| | | AEDVATYYCQQGSSIPYTFGGGTKLEIKRTVAAPSVFIFP | |
| | | PSDEQLKSGTASVVCLLNNFYPREAKVQWKVDNALQSGNS | |
| | | QESVTEQDSKDSTYSLSSTLTLSKADYEKHKVYACEVTHQ | |
| | | GLSSPVTKSFNRGEC | |
| | VL | EIVLTQSPTTMAASPGEKITITCSATSSISSNYLHWYQQK | 84 |
| | | PGFSPKLLIYRTSNLASGVPARFSGSGSGTSYSLTIGTME | |
| | | AEDVATYYCQQGSSIPYTFGGGTKLEIK | |
| | CL | RTVAAPSVFIFPPSDEQLKSGTASVVCLLNNFYPREAKVQ | 85 |
| | | WKVDNALQSGNSQESVTEQDSKDSTYSLSSTLTLSKADYE | |
| | | KHKVYACEVTHQGLSSPVTKSFNRGEC | |
| | CDR-L1 | SSISSNY | 86 |
| | CDR-L2 | RTS | 87 |
| | CDR-L3 | QGSSIPYT | 88 |
| M7 | HC | EVQLQQPGAELVRPGASVKLSCKASSYTFTRYWMNWVKQR | 89 |
| | | PEEGLEWIGMIDPYDSETHYNQKFKDKAILTVDKSSSTAY | |
| | | MQLSTLTSEDSAVYFCARSQPRYYAMDYWGQGTSVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKRVEPKSCDKTHTCPPCPAPELLGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSREEM | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | EVQLQQPGAELVRPGASVKLSCKASSYTFTRYWMNWVKQR | 90 |
| | | PEEGLEWIGMIDPYDSETHYNQKFKDKAILTVDKSSSTAY | |
| | | MQLSTLTSEDSAVYFCARSQPRYYAMDYWGQGTSVTVSS | |
| | CH | ASTKGPSVFPLAPSSKSTSGGTAALGCLVKDYFPEPVTVS | 91 |
| | | WNSGALTSGVHTFPAVLQSSGLYSLSSVVTVPSSSLGTQT | |
| | | YICNVNHKPSNTKVDKRVEPKSCDKTHTCPPCPAPELLGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 92 |
| | CDR-H2 | IDPYDSET | 93 |
| | CDR-H3 | ARSQPRYYAMDY | 94 |
| | LC | DIQMSQSPSSLSASLGERVSLTCRASQEISGFLSWLQLRP | 95 |
| | | DGTIKRLIYAASSLDSGVPKRFRGSWSGSDYSLTISSLES | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMSQSPSSLSASLGERVSLTCRASQEISGFLSWLQLRP | 96 |
| | | DGTIKRLIYAASSLDSGVPKRFRGSWSGSDYSLTISSLES | |
| | | EDFADYYCLQYSSYPYTFGGGTKLEIK | |
| | CL | RTVAAPSVFIFPPSDEQLKSGTASVVCLLNNFYPREAKVQ | 97 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 98 |
| | CDR-L2 | AAS | 99 |
| | CDR-L3 | LQYSSYPYT | 100 |
| M8 | HC | QVQLQQPGAELVKPGASVKLSCKASGYTFTDYWMHWVKQR | 101 |
| | | PGHGLEWIGEIYPSSGRANYNGNFKRKATLTVDKSSSTAY | |
| | | MQLSSLTSEDSAVYYCARSRGNYLPYWGHGTPVTVSAAST | |
| | | KGPSVFPLAPSSKSTSGGTAALGCLVKDYFPEPVTVSWNS | |
| | | GALTSGVHTFPAVLQSSGLYSLSSVVTVPSSSLGTQTYIC | |
| | | NVNHKPSNTKVDKRVEPKSCDKTHTCPPCPAPELLGGPSV | |
| | | FLFPPKPKDTLMISRTPEVTCVVVDVSHEDPEVKFNWYVD | |
| | | GVEVHNAKTKPREEQYNSTYRVVSVLTVLHQDWLNGKEYK | |
| | | CKVSNKALPAPIEKTISKAKGQPREPQVYTLPPSREEMTK | |
| | | NQVSLTCLVKGFYPSDIAVEWESNGQPENNYKTTPPVLDS | |
| | | DGSFFLYSKLTVDKSRWQQGNVFSCSVMHEALHNHYTQKS | |
| | | LSLSPGK | |
| | VH | QVQLQQPGAELVKPGASVKLSCKASGYTFTDYWMHWVKQR | 102 |
| | | PGHGLEWIGEIYPSSGRANYNGNFKRKATLTVDKSSSTAY | |
| | | MQLSSLTSEDSAVYYCARSRGNYLPYWGHGTPVTVSA | |
| | CH | ASTKGPSVFPLAPSSKSTSGGTAALGCLVKDYFPEPVTVS | 103 |
| | | WNSGALTSGVHTFPAVLQSSGLYSLSSVVTVPSSSLGTQT | |
| | | YICNVNHKPSNTKVDKRVEPKSCDKTHTCPPCPAPELLGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | GYTFTDYW | 104 |
| | CDR-H2 | IYPSSGRA | 105 |
| | CDR-H3 | ARSRGNYLPY | 106 |
| | LC | QIVLTQSPAIMSASLGERVTMTCTAGSTVSSSYLHWYQQR | 107 |
| | | PGSSPKLWIYGTSNLASGVPARFSGSGSGTSYSLTISSME | |
| | | AEDAATYYCHQYHRSPPTFGGGTKLEIKRTVAAPSVFIFP | |
| | | PSDEQLKSGTASVVCLLNNFYPREAKVQWKVDNALQSGNS | |
| | | QESVTEQDSKDSTYSLSSTLTLSKADYEKHKVYACEVTHQ | |
| | | GLSSPVTKSFNRGEC | |
| | VL | QIVLTQSPAIMSASLGERVTMTCTAGSTVSSSYLHWYQQR | 108 |
| | | PGSSPKLWIYGTSNLASGVPARFSGSGSGTSYSLTISSME | |
| | | AEDAATYYCHQYHRSPPTFGGGTKLEIK | |
| | CL | RTVAAPSVFIFPPSDEQLKSGTASVVCLLNNFYPREAKVQ | 109 |
| | | WKVDNALQSGNSQESVTEQDSKDSTYSLSSTLTLSKADYE | |
| | | KHKVYACEVTHQGLSSPVTKSFNRGEC | |
| | CDR-L1 | STVSSSY | 110 |
| | CDR-L2 | GTS | 111 |
| | CDR-L3 | HQYHRSPPT | 112 |

### Example 8: Selection of Mouse Anti-CB1 Antibodies for Humanization

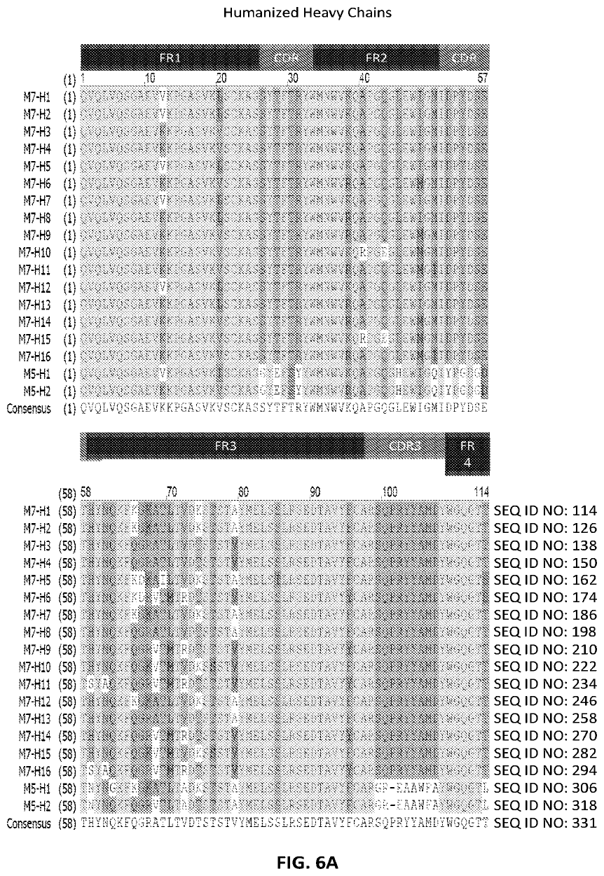

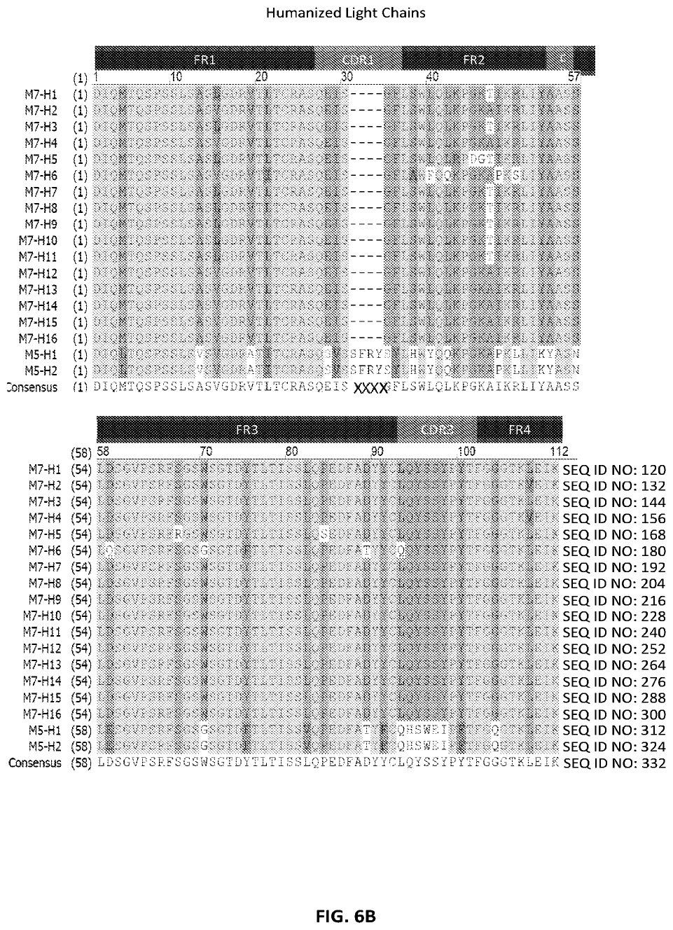

Based on the functional data, mouse anti-huCB-1 clones M7 and M5 were selected for humanization using a predictive human engineering tool derived from the PHEnon™-software package (Xoma, Berkley, CA) (U.S. Pat. No. 5,766,886). VH and VL sequences for each clone were submitted as queries and output sequences were generated based on nearest human germline matches from the Kabat database. A list of mutations in the framework region was generated to evolve the VH and VL sequences toward the human framework match. Mutational risk of individual residues was assessed through series of criteria (U.S. Pat. No. 5,766,886). Cumulatively, mutations were grouped to constitute “Low Risk” and “Medium Risk” clone pools. Output sequences and introduced mutations were validated in silico via homology modeling. Final humanized VH and VL antibody sequences were cloned into a human IgG1 vector backbone (TCAL DGV vector), expressed in CHO cells and purified by protein-A affinity chromatography according to standard methods. The sequences of the humanized clones are provided in Table 6. A consensus sequence of the heavy and light chains of the humanized M7 and M5 antibodies is provided in FIGS. 6A and 6B, respectively.

TABLE 6
Amino Acid Sequences of Humanized CB1 Antibodies

| Code | Protein | Amino Acid Sequence | SEQ |
|---|---|---|---|
| Name | Region | 1234567890123456789012345678901234567890 | ID NO |
| M7- | HC | QVQLVQSGAEVVKPGASVKLSCKASSYTFTRYWMNWVKQA | 113 |
| H1 | | PGQGLEWIGMIDPYDSETHYNQKFKGKATLTVDKSTSTAY | |
| | | MELSSLRSEDTAVYFCARSQPRYYAMDYWGQGTTVTVSS | |
| | | ASTKGPSVFPLAPSSKSTSGGTAALGCLVKDYFPEPVTVS | |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | VH | QVQLVQSGAEVVKPGASVKLSCKASSYTFTRYWMNWVKQA | 114 |
| | | PGQGLEWIGMIDPYDSETHYNQKFKGKATLTVDKSTSTAY | |
| | | MELSSLRSEDTAVYFCARSQPRYYAMDYWGQGTTVTVSS | |
| | CH | ASTKGPSVFPLAPSSKSTSGGTAALGCLVKDYFPEPVTVS | 115 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 116 |
| | CDR-H2 | IDPYDSET | 117 |
| | CDR-H3 | ARSQPRYYAMDY | 118 |
| | LC | DIQMTQSPSSLSASLGDRVTLTCRASQEISGFLSWLQLKP | 119 |
| | | GKTIKRLIYAASSLDSGVPSRFSGSWSGTDYTLTISSLQP | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASLGDRVTLTCRASQEISGFLSWLQLKP | 120 |
| | | GKTIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 121 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 122 |
| | CDR-L2 | AAS | 123 |
| | CDR-L3 | LQYSSYPYT | 124 |
| M7- | HC | QVQLVQSGAEVVKPGASVKLSCKASSYTFTRYWMNWVKQA | 125 |
| H2 | | PGQGLEWIGMIDPYDSETHYNQKFKGKATLTVDKSTSTAY | |
| | | MELSSLRSEDTAVYFCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVVKPGASVKLSCKASSYTFTRYWMNWVKQA | 126 |
| | | PGQGLEWIGMIDPYDSETHYNQKFKGKATLTVDKSTSTAY | |
| | | MELSSLRSEDTAVYFCARSQPRYYAMDYWGQGTTVTVSS | |
| | CH | ASTKGPSVFPLAPSSKSTSGGTAALGCLVKDYFPEPVTVS | 127 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 128 |
| | CDR-H2 | IDPYDSET | 129 |
| | CDR-H3 | ARSQPRYYAMDY | 130 |
| | LC | DIQMTQSPSSLSASVGDRVTLTCRASQEISGFLSWLQLKP | 131 |
| | | GKAIKRLIYAASSLDSGVPSRFSGSWSGTDYTLTISSLQP | |
| | | EDFADYYCLQYSSYPYTFGGGTKVEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASVGDRVTLTCRASQEISGFLSWLQLKP | 132 |
| | | GKAIKRLIYAASSLDSGVPSRFSGSWSGTDYTLTISSLQP | |
| | | EDFADYYCLQYSSYPYTFGGGTKVEIK | |
| | CL | RTVAAPSVFIFPPSDEQLKSGTASVVCLLNNFYPREAKVQ | 133 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 134 |
| | CDR-L2 | AAS | 135 |
| | CDR-L3 | LQYSSYPYT | 136 |
| M7- | HC | QVQLVQSGAEVKKPGASVKVSCKASSYTFTRYWMNWVKQA | 137 |
| H3 | | PGQGLEWIGMIDPYDSETHYNQKFQGRATLTVDTSTSTVY | |
| | | MELSSLRSEDTAVYFCARSQPRYYAMDYWGQGTTVTVSS | |
| | | ASTKGPSVFPLAPSSKSTSGGTAALGCLVKDYFPEPVTVS | |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | VH | QVQLVQSGAEVKKPGASVKVSCKASSYTFTRYWMNWVKQA | 138 |
| | | PGQGLEWIGMIDPYDSETHYNQKFQGRATLTVDTSTSTVY | |
| | | MELSSLRSEDTAVYFCARSQPRYYAMDYWGQGTTVTVSS | |
| | CH | ASTKGPSVFPLAPSSKSTSGGTAALGCLVKDYFPEPVTVS | 139 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 140 |
| | CDR-H2 | IDPYDSET | 141 |
| | CDR-H3 | ARSQPRYYAMDY | 142 |
| | LC | DIQMTQSPSSLSASLGDRVTLTCRASQEISGFLSWLQLKP | 143 |
| | | GKTIKRLIYAASSLDSGVPSRFSGSWSGTDYTLTISSLQP | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASLGDRVTLTCRASQEISGFLSWLQLKP | 144 |
| | | GKTIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 145 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 146 |
| | CDR-L2 | AAS | 147 |
| | CDR-L3 | LQYSSYPYT | 148 |
| M7- | HC | QVQLVQSGAEVKKPGASVKVSCKASSYTFTRYWMNWVKQA | 149 |
| H4 | | PGQGLEWIGMIDPYDSETHYNQKFQGRATLTVDTSTSTVY | |
| | | MELSSLRSEDTAVYFCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVKKPGASVKVSCKASSYTFTRYWMNWVRQA | 150 |
| | | PGQGLEWMGMIDPYDSETHYNQKFKDRVTMTRDTSTSTVY | |
| | | MELSSLRSEDTAVYYCARSQPRYYAMDYWGQGTSVTVSS | |
| | CH | ASTKGPSVFPLAPSSKSTSGGTAALGCLVKDYFPEPVTVS | 151 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 152 |
| | CDR-H2 | IDPYDSET | 153 |
| | CDR-H3 | ARSQPRYYAMDY | 154 |
| | LC | DIQMTQSPSSLSASVGDRVTLTCRASQEISGFLSWLQLKP | 155 |
| | | GKAIKRLIYAASSLDSGVPSRFSGSWSGTDYTLTISSLQP | |
| | | EDFADYYCLQYSSYPYTFGGGTKVEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASVGDRVTLTCRASQEISGFLSWLQLKP | 156 |
| | | GKAIKRLIYAASSLDSGVPSRFSGSWSGTDYTLTISSLQP | |
| | | EDFADYYCLQYSSYPYTFGGGTKVEIK | |
| | CL | RTVAAPSVFIFPPSDEQLKSGTASVVCLLNNFYPREAKVQ | 157 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 158 |
| | CDR-L2 | AAS | 159 |
| | CDR-L3 | QYSSYPYT | 160 |
| M7- | HC | QVQLVQSGAEVVKPGASVKLSCKASSYTFTRYWMNWVKQA | 161 |
| H5 | | PGQGLEWIGMIDPYDSETHYNQKFKDKAILTVDKSTSTAY | |
| | | MELSTLRSEDTAVYFCARSQPRYYAMDYWGQGTSVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVVKPGASVKLSCKASSYTFTRYWMNWVKQA | 162 |
| | | PGQGLEWIGMIDPYDSETHYNQKFKDKAILTVDKSTSTAY | |
| | | MELSTLRSEDTAVYFCARSQPRYYAMDYWGQGTSVTVSS | |
| | CH | ASTKGPSVFPLAPSSKSTSGGTAALGCLVKDYFPEPVTVS | 163 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 164 |
| | CDR-H2 | IDPYDSET | 165 |
| | CDR-H3 | ARSQPRYYAMDY | 166 |
| | LC | DIQMTQSPSSLSASLGDRVTLTCRASQEISGFLSWLQLRP | 167 |
| | | DGTIKRLIYAASSLDSGVPSRFRGSWSGTDYTLTISSLQS | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASLGDRVTLTCRASQEISGFLSWLQLRP | 168 |
| | | DGTIKRLIYAASSLDSGVPSRFRGSWSGTDYTLTISSLQS | |
| | | EDFADYYCLQYSSYPYTFGGGTKLEIK | |
| | CL | RTVAAPSVFIFPPSDEQLKSGTASVVCLLNNFYPREAKVQ | 169 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 170 |
| | CDR-L2 | AAS | 171 |
| | CDR-L3 | LQYSSYPYT | 172 |
| M7- | HC | QVQLVQSGAEVKKPGASVKVSCKASSYTFTRYWMNWVRQA | 173 |
| H6 | | PGQGLEWMGMIDPYDSETHYNQKFKDRVTMTRDTSTSTVY | |
| | | MELSSLRSEDTAVYYCARSQPRYYAMDYWGQGTSVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKRVEPKSCDKTHTCPPCPAPELLGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSREEM | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVKKPGASVKVSCKASSYTFTRYWMNWVRQA | 174 |
| | | PGQGLEWMGMIDPYDSETHYNQKFKDRVTMTRDTSTSTVY | |
| | | MELSSLRSEDTAVYYCARSQPRYYAMDYWGQGTSVTVSS | |
| | CH | ASTKGPSVFPLAPSSKSTSGGTAALGCLVKDYFPEPVTVS | 175 |
| | | WNSGALTSGVHTFPAVLQSSGLYSLSSVVTVPSSSLGTQT | |
| | | YICNVNHKPSNTKVDKRVEPKSCDKTHTCPPCPAPELLGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 176 |
| | CDR-H2 | IDPYDSET | 177 |
| | CDR-H3 | ARSQPRYYAMDY | 178 |
| | LC | DIQMTQSPSSLSASVGDRVTITCRASQEISGFLAWFQQKP | 179 |
| | | GKAPKSLIYAASSLQSGVPSRFSGSGSGTDFTLTISSLQP | |
| | | EDFATYYCQQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASVGDRVTITCRASQEISGFLAWFQQKP | 180 |
| | | GKAPKSLIYAASSLQSGVPSRFSGSGSGTDFTLTISSLQP | |
| | | EDFATYYCQQYSSYPYTFGGGTKLEIK | |
| | CL | RTVAAPSVFIFPPSDEQLKSGTASVVCLLNNFYPREAKVQ | 181 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 182 |
| | CDR-L2 | AAS | 183 |
| | CDR-L3 | QQYSSYPYT | 184 |
| M7- | HC | QVQLVQSGAEVVKPGASVKLSCKASSYTFTRYWMNWVKQA | 185 |
| H7 | | PGQGLEWIGMIDPYDSETHYNQKFKGKATLTVDKSTSTAY | |
| | | MELSSLRSEDTAVYFCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVVKPGASVKLSCKASSYTFTRYWMNWVKQA | 186 |
| | | PGQGLEWIGMIDPYDSETHYNQKFKGKATLTVDKSTSTAY | |
| | | MELSSLRSEDTAVYFCARSQPRYYAMDYWGQGTTVTVSS | |
| | CH | ASTKGPSVFPLAPSSKSTSGGTAALGCLVKDYFPEPVTVS | 187 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 188 |
| | CDR-H2 | IDPYDSET | 189 |
| | CDR-H3 | ARSQPRYYAMDY | 190 |
| | LC | DIQMTQSPSSLSASLGDRVTLTCRASQEISGFLSWLQLKP | 191 |
| | | GKTIKRLIYAASSLDSGVPSRFSGSWSGTDYTLTISSLQP | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASLGDRVTLTCRASQEISGFLSWLQLKP | 192 |
| | | GKTIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 193 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 194 |
| | CDR-L2 | AAS | 195 |
| | CDR-L3 | LQYSSYPYT | 196 |
| M7- | HC | QVQLVQSGAEVKKPGASVKLSCKASSYTFTRYWMNWVRQA | 197 |
| H8 | | PGQGLEWIGMIDPYDSETHYNQKFQGRATLTVDTSTSTAY | |
| | | MELSSLRSEDTAVYFCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVKKPGASVKLSCKASSYTFTRYWMNWVRQA | 198 |
| | | PGQGLEWIGMIDPYDSETHYNQKFQGRATLTVDTSTSTAY | |
| | | MELSSLRSEDTAVYFCARSQPRYYAMDYWGQGTTVTVSS | |
| | CH | ASTKGPSVFPLAPSSKSTSGGTAALGCLVKDYFPEPVTVS | 199 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 200 |
| | CDR-H2 | IDPYDSET | 201 |
| | CDR-H3 | ARSQPRYYAMDY | 202 |
| | LC | DIQMTQSPSSLSASLGDRVTLTCRASQEISGFLSWLQLKP | 203 |
| | | GKTIKRLIYAASSLDSGVPSRFSGSWSGTDYTLTISSLQP | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASLGDRVTLTCRASQEISGFLSWLQLKP | 204 |
| | | GKTIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 205 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 206 |
| | CDR-L2 | AAS | 207 |
| | CDR-L3 | LQYSSYPYT | 208 |
| M7- | HC | QVQLVQSGAEVKKPGASVKVSCKASSYTFTRYWMNWVRQA | 209 |
| H9 | | PGQGLEWMGMIDPYDSETHYNQKFQGRVTMTRDTSTSTVY | |
| | | MELSSLRSEDTAVYYCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVKKPGASVKVSCKASSYTFTRYWMNWVRQA | 210 |
| | | PGQGLEWMGMIDPYDSETHYNQKFQGRVTMTRDTSTSTVY | |
| | | MELSSLRSEDTAVYYCARSQPRYYAMDYWGQGTTVTVSS | |
| | CH | ASTKGPSVFPLAPSSKSTSGGTAALGCLVKDYFPEPVTVS | 211 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 212 |
| | CDR-H2 | IDPYDSET | 213 |
| | CDR-H3 | ARSQPRYYAMDY | 214 |
| | LC | DIQMTQSPSSLSASLGDRVTLTCRASQEISGFLSWLQLKP | 215 |
| | | GKTIKRLIYAASSLDSGVPSRFSGSWSGTDYTLTISSLQP | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASLGDRVTLTCRASQEISGFLSWLQLKP | 216 |
| | | GKTIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 217 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 218 |
| | CDR-L2 | AAS | 219 |
| | CDR-L3 | LQYSSYPYT | 220 |
| M7- | HC | QVQLVQSGAEVKKPGASVKVSCKASSYTFTRYWMNWVKQR | 221 |
| H10 | | PGEGLEWMGMIDPYDSETHYNQKFQGKVTMTVDKSSSTVY | |
| | | MELSSLRSEDTAVYFCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVKKPGASVKVSCKASSYTFTRYWMNWVKQR | 222 |
| | | PGEGLEWMGMIDPYDSETHYNQKFQGKVTMTVDKSSSTVY | |
| | | MELSSLRSEDTAVYFCARSQPRYYAMDYWGQGTTVTVSS | |
| | CH | ASTKGPSVFPLAPSSKSTSGGTAALGCLVKDYFPEPVTVS | 223 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 224 |
| | CDR-H2 | IDPYDSET | 225 |
| | CDR-H3 | ARSQPRYYAMDY | 226 |
| | LC | DIQMTQSPSSLSASLGDRVTLTCRASQEISGFLSWLQLKP | 227 |
| | | GKTIKRLIYAASSLDSGVPSRFSGSWSGTDYTLTISSLQP | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASLGDRVTLTCRASQEISGFLSWLQLKP | 228 |
| | | GKTIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 229 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 230 |
| | CDR-L2 | AAS | 231 |
| | CDR-L3 | LQYSSYPYT | 232 |
| M7- | HC | QVQLVQSGAEVKKPGASVKVSCKASSYTFTRYWMNWVRQA | 233 |
| H11 | | PGQGLEWMGIIDPYDSETSYAQKFQGRVTMTRDTSTSTVY | |
| | | MELSSLRSEDTAVYYCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVKKPGASVKVSCKASSYTFTRYWMNWVRQA | 234 |
| | | PGQGLEWMGIIDPYDSETSYAQKFQGRVTMTRDTSTSTVY | |
| | | MELSSLRSEDTAVYYCARSQPRYYAMDYWGQGTTVTVSS | |
| | CH | ASTKGPSVFPLAPSSKSTSGGTAALGCLVKDYFPEPVTVS | 235 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 236 |
| | CDR-H2 | IDPYDSET | 237 |
| | CDR-H3 | ARSQPRYYAMDY | 238 |
| | LC | DIQMTQSPSSLSASLGDRVTLTCRASQEISGFLSWLQLKP | 239 |
| | | GKTIKRLIYAASSLDSGVPSRFSGSWSGTDYTLTISSLQP | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASLGDRVTLTCRASQEISGFLSWLQLKP | 240 |
| | | GKTIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 241 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 242 |
| | CDR-L2 | AAS | 243 |
| | CDR-L3 | LQYSSYPYT | 244 |
| M7- | HC | QVQLVQSGAEVVKPGASVKLSCKASSYTFTRYWMNWVKQA | 245 |
| H12 | | PGQGLEWIGMIDPYDSETHYNQKFKGKATLTVDKSTSTAY | |
| | | MELSSLRSEDTAVYFCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVVKPGASVKLSOKASSYTFTRYWMNWVKQA | 246 |
| | | PGQGLEWIGMIDPYDSETHYNQKFKGKATLTVDKSTSTAY | |
| | | MELSSLRSEDTAVYFCARSQPRYYAMDYWGQGTTVTVSS | |
| | CH | ASTKGPSVFPLAPSSKSTSGGTAALGCLVKDYFPEPVTVS | 247 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 248 |
| | CDR-H2 | IDPYDSET | 249 |
| | CDR-H3 | ARSQPRYYAMDY | 250 |
| | LC | DIQMTQSPSSLSASVGDRVTLTCRASQEISGFLSWLQLKP | 251 |
| | | GKAIKRLIYAASSLDSGVPSRFSGSWSGTDYTLTISSLQP | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASVGDRVTLTCRASQEISGFLSWLQLKP | 252 |
| | | GKAIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 253 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 254 |
| | CDR-L2 | AAS | 255 |
| | CDR-L3 | LQYSSYPYT | 256 |
| M7- | HC | QVQLVQSGAEVKKPGASVKLSCKASSYTFTRYWMNWVRQA | 257 |
| H13 | | PGQGLEWIGMIDPYDSETHYNQKFQGRATLTVDTSTSTAY | |
| | | MELSSLRSEDTAVYFCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVKKPGASVKLSCKASSYTFTRYWMNWVRQA | 258 |
| | | PGQGLEWIGMIDPYDSETHYNQKFQGRATLTVDTSTSTAY | |
| | | MELSSLRSEDTAVYFCARSQPRYYAMDYWGQGTTVTVSS | |
| | CH | ASTKGPSVFPLAPSSKSTSGGTAALGCLVKDYFPEPVTVS | 259 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 260 |
| | CDR-H2 | IDPYDSET | 261 |
| | CDR-H3 | ARSQPRYYAMDY | 262 |
| | LC | DIQMTQSPSSLSASVGDRVTLTCRASQEISGFLSWLQLKP | 263 |
| | | GKAIKRLIYAASSLDSGVPSRFSGSWSGTDYTLTISSLQP | |
| | | EDFADYYCLQYSSYPYTFGGGTKLEIK | |
| | | RTVAAPSVFIFPPSDEQLKSGTASVVCLLNNFYPREAKVQ | |
| | | WKVDNALQSGNSQESVTEQDSKDSTYSLSSTLTLSKADYE | |
| | | KHKVYACEVTHQGLSSPVTKSFNRGEC | |
| | VL | DIQMTQSPSSLSASVGDRVTLTCRASQEISGFLSWLQLKP | 264 |
| | | GKAIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 265 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 266 |
| | CDR-L2 | AAS | 267 |
| | CDR-L3 | LQYSSYPYT | 268 |
| M7- | HC | QVQLVQSGAEVKKPGASVKVSCKASSYTFTRYWMNWVRQA | 269 |
| H14 | | PGQGLEWMGMIDPYDSETHYNQKFQGRVTMTRDTSTSTVY | |
| | | MELSSLRSEDTAVYYCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVKKPGASVKVSCKASSYTFTRYWMNWVRQA | 270 |
| | | PGQGLEWMGMIDPYDSETHYNQKFQGRVTMTRDTSTSTVY | |
| | | MELSSLRSEDTAVYYCARSQPRYYAMDYWGQGTTVTVSS | |
| | CH | ASTKGPSVFPLAPSSKSTSGGTAALGCLVKDYFPEPVTVS | 271 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 272 |
| | CDR-H2 | IDPYDSET | 273 |
| | CDR-H3 | ARSQPRYYAMDY | 274 |
| | LC | DIQMTQSPSSLSASVGDRVTLTCRASQEISGFLSWLQLKP | 275 |
| | | GKAIKRLIYAASSLDSGVPSRFSGSWSGTDYTLTISSLQP | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASVGDRVTLTCRASQEISGFLSWLQLKP | 276 |
| | | GKAIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 277 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 278 |
| | CDR-L2 | AAS | 279 |
| | CDR-L3 | LQYSSYPYT | 280 |
| M7- | HC | QVQLVQSGAEVKKPGASVKVSCKASSYTFTRYWMNWVKQR | 281 |
| H15 | | PGEGLEWMGMIDPYDSETHYNQKFQGKVTMTVDKSSSTVY | |
| | | MELSSLRSEDTAVYFCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVKKPGASVKVSCKASSYTFTRYWMNWVKQR | 282 |
| | | PGEGLEWMGMIDPYDSETHYNQKFQGKVTMTVDKSSSTVY | |
| | | MELSSLRSEDTAVYFCARSQPRYYAMDYWGQGTTVTVSS | |
| | CH | ASTKGPSVFPLAPSSKSTSGGTAALGCLVKDYFPEPVTVS | 283 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 284 |
| | CDR-H2 | IDPYDSET | 285 |
| | CDR-H3 | ARSQPRYYAMDY | 286 |
| | LC | DIQMTQSPSSLSASVGDRVTLTCRASQEISGFLSWLQLKP | 287 |
| | | GKAIKRLIYAASSLDSGVPSRFSGSWSGTDYTLTISSLQP | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASVGDRVTLTCRASQEISGFLSWLQLKP | 288 |
| | | GKAIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 289 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 290 |
| | CDR-L2 | AAS | 291 |
| | CDR-L3 | LQYSSYPYT | 292 |
| M7- | HC | QVQLVQSGAEVKKPGASVKVSCKASSYTFTRYWMNWVRQA | 293 |
| H16 | | PGQGLEWMGIIDPYDSETSYAQKFQGRVTMTRDTSTSTVY | |
| | | MELSSLRSEDTAVYYCARSQPRYYAMDYWGQGTTVTVSSA | |
| | | STKGPSVFPLAPSSKSTSGGTAALGCLVKDYFPEPVTVSW | |
| | | NSGALTSGVHTFPAVLQSSGLYSLSSVVTVPSSSLGTQTY | |
| | | ICNVNHKPSNTKVDKKVEPKSCDKTHTCPPCPAPEAAGGP | |
| | | SVFLFPPKPKDTLMISRTPEVTCVVVDVSHEDPEVKFNWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | VH | QVQLVQSGAEVKKPGASVKVSCKASSYTFTRYWMNWVRQA | 294 |
| | | PGQGLEWMGIIDPYDSETSYAQKFQGRVTMTRDTSTSTVY | |
| | | MELSSLRSEDTAVYYCARSQPRYYAMDYWGQGTTVTVSS | |
| | CH | ASTKGPSVFPLAPSSKSTSGGTAALGCLVKDYFPEPVTVS | 295 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | SYTFTRYW | 296 |
| | CDR-H2 | IDPYDSET | 297 |
| | CDR-H3 | ARSQPRYYAMDY | 298 |
| | LC | DIQMTQSPSSLSASVGDRVTLTCRASQEISGFLSWLQLKP | 299 |
| | | GKAIKRLIYAASSLDSGVPSRFSGSWSGTDYTLTISSLQP | |
| | | EDFADYYCLQYSSYPYTFGGGTKLEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | VL | DIQMTQSPSSLSASVGDRVTLTCRASQEISGFLSWLQLKP | 300 |
| | | GKAIKRLIYAASSLDSGVPSRFSGSWSGTDYTLTISSLQP | |
| | | EDFADYYCLQYSSYPYTFGGGTKLEIK | |
| | CL | RTVAAPSVFIFPPSDEQLKSGTASVVCLLNNFYPREAKVQ | 301 |
| | | WKVDNALQSGNSQESVTEQDSKDSTYSLSSTLTLSKADYE | |
| | | KHKVYACEVTHQGLSSPVTKSFNRGEC | |
| | CDR-L1 | QEISGF | 302 |
| | CDR-L2 | AAS | 303 |
| | CDR-L3 | LQYSSYPYT | 304 |
| M5- | HC | QVQLVQSGAEVVKPGASVKISCKASGYEFSYYWMNWVKQA | 305 |
| H1 | | PGQGHEWIGQIYPGDGDTNYNGKFKGKATLTADKSTSTAY | |
| | | MELSSLRSEDTAVYFCARGREAAWFAYWGQGTLVTVSSAS | |
| | | TKGPSVFPLAPSSKSTSGGTAALGCLVKDYFPEPVTVSWN | |
| | | SGALTSGVHTFPAVLQSSGLYSLSSVVTVPSSSLGTQTYI | |
| | | CNVNHKPSNTKVDKKVEPKSCDKTHTCPPCPAPEAAGGPS | |
| | | VFLFPPKPKDTLMISRTPEVTCVVVDVSHEDPEVKFNWYV | |
| | | DGVEVHNAKTKPREEQYNSTYRVVSVLTVLHQDWLNGKEY | |
| | | KCKVSNKALPAPIEKTISKAKGQPREPQVYTLPPSRDELT | |
| | | KNQVSLTCLVKGFYPSDIAVEWESNGQPENNYKTTPPVLD | |
| | | SDGSFFLYSKLTVDKSRWQQGNVFSCSVMHEALHNHYTQK | |
| | | SLSLSPGK | |
| | VH | QVQLVQSGAEVVKPGASVKISCKASGYEFSYYWMNWVKQA | 306 |
| | | PGQGHEWIGQIYPGDGDTNYNGKFKGKATLTADKSTSTAY | |
| | | MELSSLRSEDTAVYFCARGREAAWFAYWGQGTLVTVSS | |
| | CH | ASTKGPSVFPLAPSSKSTSGGTAALGCLVKDYFPEPVTVS | 307 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | GYEFSYYW | 308 |
| | CDR-H2 | IYPGDGDT | 309 |
| | CDR-H3 | ARGREAAWFAY | 310 |
| | LC | DIQLTQSPSSLSVSVGDRATITCRASQSVSSFRYSYLHWY | 311 |
| | | QQKPGKAPKLLIKYASNLESGVPSRFSGSGSGTDFTLTIS | |
| | | SVQPEDFATYFCQHSWEIPFTFGQGTKLEIKRTVAAPSVF | |
| | | IFPPSDEQLKSGTASVVCLLNNFYPREAKVQWKVDNALQS | |
| | | GNSQESVTEQDSKDSTYSLSSTLTLSKADYEKHKVYACEV | |
| | | THQGLSSPVTKSFNRGEC | |
| | VL | DIQLTQSPSSLSVSVGDRATITCRASQSVSSFRYSYLHWY | 312 |
| | | QQKPGKAPKLLIKYASNLESGVPSRFSGSGSGTDFTLTIS | |
| | | SVQPEDFATYFCQHSWEIPFTFGQGTKLEIK | |
| | CL | RTVAAPSVFIFPPSDEQLKSGTASVVCLLNNFYPREAKVQ | 313 |
| | | WKVDNALQSGNSQESVTEQDSKDSTYSLSSTLTLSKADYE | |
| | | KHKVYACEVTHQGLSSPVTKSFNRGEC | |
| | CDR-L1 | QSVSSFRYSY | 314 |
| | CDR-L2 | YAS | 315 |
| | CDR-L3 | QHSWEIPFT | 316 |
| M5- | HC | QVQLVQSGAEVKKPGASVKVSCKASGYEFSYYWMNWVRQA | 317 |
| H2 | | PGQGHEWIGQIYPGDGDTNYNQKFQGRVTLTADTSTSTAY | |
| | | MELSSLRSEDTAVYFCARGREAAWFAYWGQGTLVTVSSAS | |
| | | TKGPSVFPLAPSSKSTSGGTAALGCLVKDYFPEPVTVSWN | |
| | | SGALTSGVHTFPAVLQSSGLYSLSSVVTVPSSSLGTQTYI | |
| | | CNVNHKPSNTKVDKKVEPKSCDKTHTCPPCPAPEAAGGPS | |
| | | VFLFPPKPKDTLMISRTPEVTCVVVDVSHEDPEVKFNWYV | |
| | | DGVEVHNAKTKPREEQYNSTYRVVSVLTVLHQDWLNGKEY | |
| | | KCKVSNKALPAPIEKTISKAKGQPREPQVYTLPPSRDELT | |
| | | KNQVSLTCLVKGFYPSDIAVEWESNGQPENNYKTTPPVLD | |
| | | SDGSFFLYSKLTVDKSRWQQGNVFSCSVMHEALHNHYTQK | |
| | | SLSLSPGK | |
| | VH | QVQLVQSGAEVKKPGASVKVSCKASGYEFSYYWMNWVRQA | 318 |
| | | PGQGHEWIGQIYPGDGDTNYNQKFQGRVTLTADTSTSTAY | |
| | | MELSSLRSEDTAVYFCARGREAAWFAYWGQGTLVTVSS | |
| | CH | ASTKGPSVFPLAPSSKSTSGGTAALGCLVKDYFPEPVTVS | 319 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKFNW | |
| | | YVDGVEVHNAKTKPREEQYNSTYRVVSVLTVLHQDWLNGK | |
| | | EYKCKVSNKALPAPIEKTISKAKGQPREPQVYTLPPSRDE | |
| | | LTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | CDR-H1 | GYEFSYYW | 320 |
| | CDR-H2 | IYPGDGDT | 321 |
| | CDR-H3 | ARGREAAWFAY | 322 |
| | LC | DIQLTQSPSSLSVSVGDRATITCRASQSVSSFRYSYLHWY | 323 |
| | | QQKPGKAPKLLIKYASNLESGVPSRFSGSGSGTDFTLTIS | |
| | | SVQPEDFATYFCQHSWEIPFTFGQGTKLEIKRTVAAPSVF | |
| | | IFPPSDEQLKSGTASVVCLLNNFYPREAKVQWKVDNALQS | |
| | | GNSQESVTEQDSKDSTYSLSSTLTLSKADYEKHKVYACEV | |
| | | THQGLSSPVTKSFNRGEC | |
| | VL | DIQLTQSPSSLSVSVGDRATITCRASQSVSSFRYSYLHWY | 324 |
| | | QQKPGKAPKLLIKYASNLESGVPSRFSGSGSGTDFTLTIS | |
| | | SVQPEDFATYFCQHSWEIPFTFGQGTKLEIK | |
| | CL | RTVAAPSVFIFPPSDEQLKSGTASVVCLLNNFYPREAKVQ | 325 |
| | | WKVDNALQSGNSQESVTEQDSKDSTYSLSSTLTLSKADYE | |
| | | KHKVYACEVTHQGLSSPVTKSFNRGEC | |
| | CDR-L1 | QSVSSFRYSY | 326 |
| | CDR-L2 | YAS | 327 |
| | CDR-L3 | QHSWEIPFT | 328 |

### Example 9: Re-Evaluation of the Humanized Anti-CB1 Antibodies in Cell Binding and Functional Assays

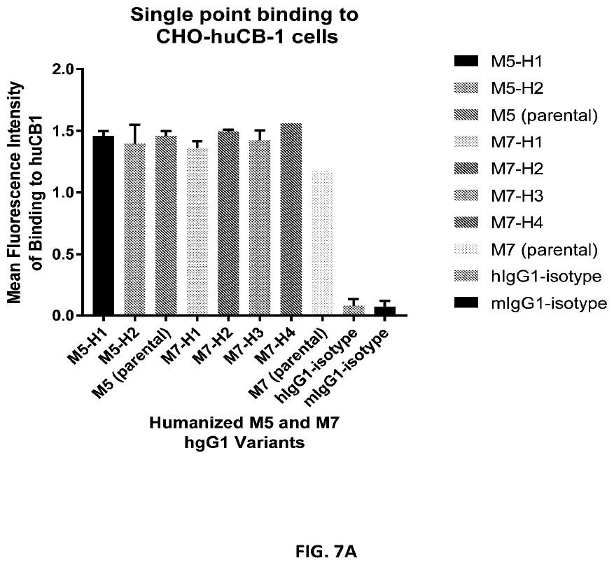

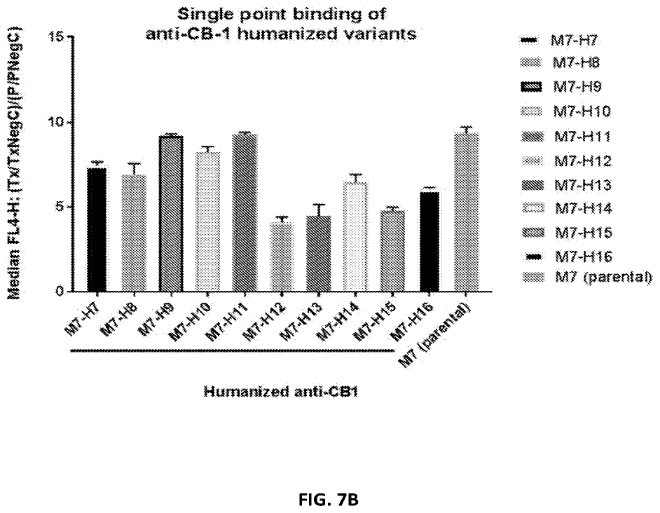

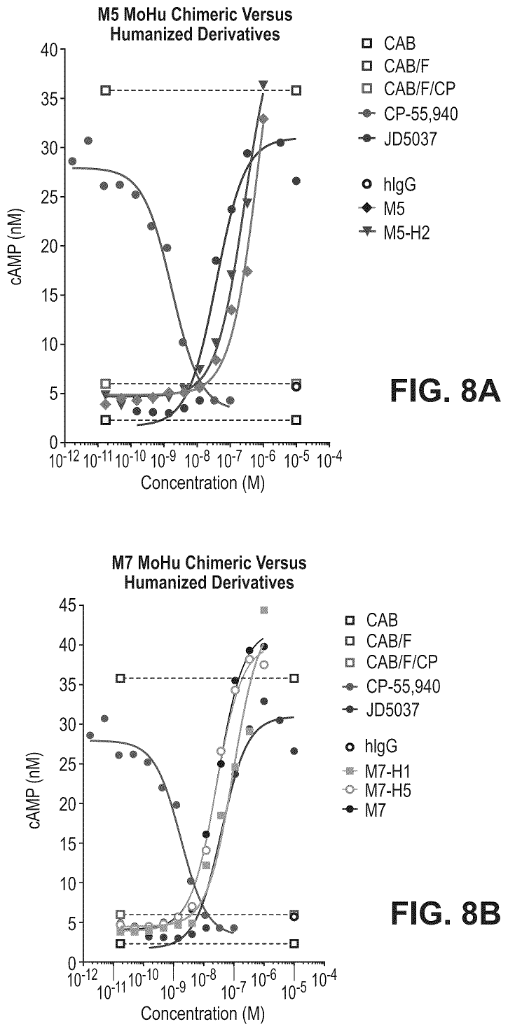

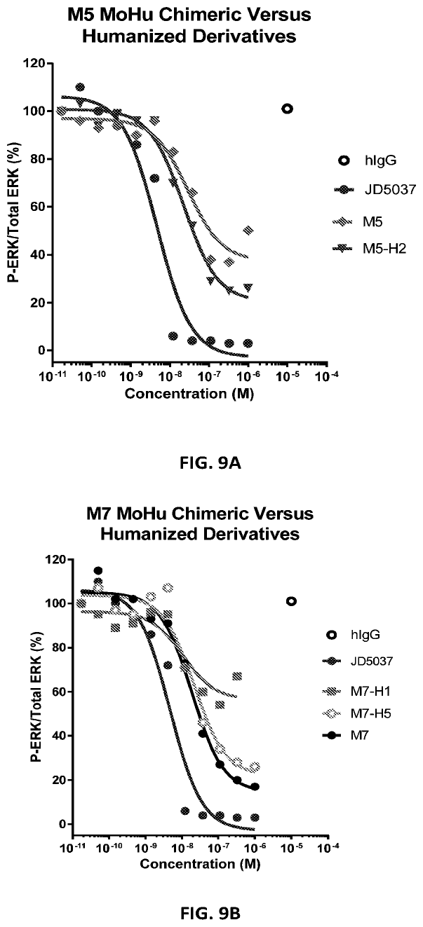

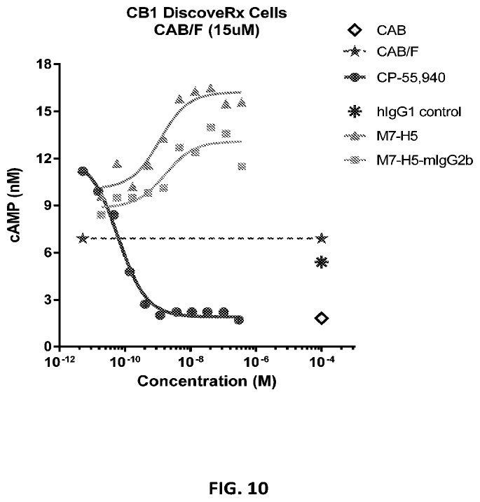

The humanized anti-CB1 variants were reevaluated for their ability to bind CHO-huCB-1 cells and compared to mouse parental clones M5 and M7 (FIGS. 7A and 7B) to ensure that binding was retained. The assay conditions used were similar to those described in Example 5. Following binding analysis, test variants were also evaluated for function as antagonists in the cAMP and pERK assays according to Examples 3 and 4 (FIGS. 8A, 8B, 9A, and 9B) to ensure that binding and activity was maintained after humanization. All clones retained their binding and antagonist activity. FIG. 10 shows the inverse agonism displayed by humanized anti-CB1 Ab M7-H5 and an IgG2b formats.

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. An isolated antibody or antigen-binding fragment thereof that binds human cannabinoid type 1 receptor (CB1)(SEQ ID NO:1), wherein the antibody or antigen-binding fragment comprises a variable heavy (VH) domain sequence and a variable light (VL) domain sequence, and wherein the antibody or antigen-binding fragment comprises six complementarity determining regions (CDRs): CDR-H1, CDR-H2, CDR-H3, CDR-L1, CDR-L2, and CDR-L3, wherein
CDR-H1 has an amino acid sequence G-Y-E-F-S-Y-Y-W (SEQ ID NO: 308);
CDR-H2 has an amino acid sequence I-Y-P-G-D-G-D-T (SEQ ID NO: 309);
CDR-H3 has an amino acid sequence A-R-G-R-E-A-A-W-F-A-Y (SEQ ID NO: 310);
CDR-L1 has an amino acid sequence Q-S-V-S-S-F-R-Y-S-Y (SEQ ID NO: 314) ;
CDR-L2 has an amino acid sequence: Y-A-S; and
CDR-L3 has an amino acid sequence: Q-H-S-W-E-I-P-F-T (SEQ ID NO: 316).

2-3. (canceled)

4. The isolated antibody or antigen-binding fragment thereof of claim 1, wherein the antibody or antigen-binding fragment thereof comprises the the VH/VL pair selected from the group consisting of SEQ ID NOs: 306/312 and 318/324.

5-6. (canceled)

7. The isolated antibody or antigen-binding fragment thereof of claim 1, wherein the antibody or antigen-binding fragment thereof comprises the (HCDR set/LCDR set) pair selected from the group consisting of (SEQ ID NO: 308, SE ID NO: 309, SEQ ID NO: 310/SEQ ID NO: 314, Y-A-S, SEQ ID NO: 316) and (SEQ ID NO: 320, SEQ ID NO: 321, SEQ ID NO: 322/SEQ ID NO: 326, Y-A-S, SEQ ID NO: 328).

8. The isolated antibody or antigen-binding fragment thereof of claim 7, wherein the antibody or antigen-binding fragment thereof comprises the HC/LC pair selected from the group consisting of SEQ ID NOs: 305/311 and 317/323.

9. The isolated antibody or antigen-binding fragment thereof of claim 1, wherein the VH domain sequence has at least 95% identity to an amino acid sequence selected from the group consisting of SEQ ID NOs: 306 and 318 and the VL domain sequence has at least 95% identity to an amino acid sequence selected from the group consisting of SEQ ID NOs: 312 and 324.

10-25. (canceled)

26. The isolated antibody or antigen-binding fragment thereof of claim 9, wherein the VH is set forth in SEQ ID NO: 306 and the VL is set forth in SEQ ID NO: 312.

27. The isolated antibody or antigen-binding fragment thereof of claim 9, wherein the VH is set forth in SEQ ID NO: 318 and the VL is set forth in SEQ ID NO: 324.

28. (canceled)

29. The isolated antibody or antigen-binding fragment thereof of claim 1, wherein the fragment comprises a Fab fragment, a Fab′ fragment, a F(ab)₂ fragment or a scFv fragment.

30. The isolated antibody or antigen-binding fragment thereof of claim 1, wherein the antibody or antigen-binding fragment thereof comprises a human Fc region selected from the group consisting of an IgG1, IgG2, IgG3, IgG4, IgA, IgD, IgE, and IgM Fc.

31. The isolated antibody or antigen-binding fragment thereof of claim 1, wherein the antibody or antigen-binding fragment thereof comprises a modified human Fc region.

32. The isolated antibody or antigen-binding fragment thereof of claim 31, wherein the antibody or antigen-binding fragment thereof comprises a modified human Fc region comprising a mutation selected from the group consisting of L234A/L235A, S228P, A330S, P331S, E233P/L234V/L235A, A327G/A330S/P331S, L234F/L235E/P331S, and N297Q.

33. A multispecific binding protein comprising an antigen-binding fragment of claim 1.

34-36. (canceled)

37. The isolated antibody or antigen-binding fragment thereof of claim 1, wherein the antibody or antigen-binding fragment thereof is a humanized antibody.

38-47. (canceled)

48. An isolated nucleic acid molecule encoding the antibody or antigen-binding fragment thereof of claim 1.

49. An expression vector comprising the nucleic acid molecule of claim 48.

50. A host cell comprising the expression vector of claim 49.

51. A method of modulating CB1 signaling, the method comprising contacting a cell expressing CB1 with the antibody or antigen-binding fragment thereof of claim 1.

52-54. (canceled)

55. A pharmaceutical composition comprising an isolated antibody or antigen-binding fragment thereof of claim 1 and at least one pharmaceutically acceptable excipient.

56. A method for inhibiting the biological activity of CB1 in a subject in need thereof, the method comprising administering an effective amount of the pharmaceutical composition of claim 55 to the subject thereby inhibiting the activity of the CB1 protein in the subject.

57-62. (canceled)

63. An antibody conjugate comprising the isolated antibody or antigen-binding fragment thereof of claim 1, wherein the antibody or antigen-binding fragment thereof is conjugated to an agent selected from the group consisting of a therapeutic agent, a cytotoxic agent, an immunoadhesion molecule, and an imaging agent.
